(19) United States
(12) Patent Application Publication
(10) Pub. No.: US 2012/0121555 A1
(43) Pub. Date: May 17, 2012

(54) Compositions and Methods of Using Living and Non-Living Bioactive Devices with Components Derived From Self-Renewing Colony Forming Cells Cultured and Expanded In Vitro

(75) Inventors: Gene KOPEN, Wynnewood, PA (US); Vanessa Ragaglia, Newtown Square, PA (US); Candace Brayfield, Worchester, MA (US)

(73) Assignee: Garnet Biotherapeutics, Inc., Malvern, PA (US)

(21) Appl. No.: 13/349,379

(22) Filed: Jan. 12, 2012

Related U.S. Application Data
(63) Continuation of application No. 13/118,228, filed on May 27, 2011.
(60) Provisional application No. 61/349,661, filed on May 28, 2010.

(51) Int. Cl. A61K 35/28; A61K 38/18; A61P 19/04; C12N 5/071

(52) U.S. Cl. 424/93.7; 435/325; 514/8.1

(57) ABSTRACT

The invention relates to methods and uses of cells for the prevention and treatment of a wide variety of diseases and disorders and the repair and regeneration of tissues and organs using low passage and extensively passaged in vitro cultured, self-renewing, colony forming somatic cells (CF-SC). For example, adult bone marrow-derived somatic cells (ABM-SC), or compositions produced by such cells, are useful alone or in combination with other components for treating, for example, cardiovascular, neurological, integumentary, dermatological, periodontal, and immune mediated diseases, disorders, pathologies, and injuries.

### BACKGROUND OF THE INVENTION

1. Field of the Invention

The present invention relates generally to the generation and use of in vitro cultured self-renewing colony forming somatic cells (CF-SC), and compositions produced by such cells, for the treatment of a variety of diseases and disorders. One example of such CF-SC are adult human bone marrow-derived somatic cells (hABM-SC).

The present invention also relates to manipulation of CF-SC cell populations during cultivation to modulate (i.e., up- or down-regulate) production of various soluble or secreted compositions produced by in vitro cultured and expanded self-renewing colony forming cells.

The field of the invention also relates to cell-based and tissue-engineering therapies; particularly, methods of using and/or administering CF-SC, or compositions produced by such cells, including administration via incorporation in, or mixture with, pharmaceutically acceptable carriers (such as a pharmaceutically acceptable solution or a transient, permanent, or biodegradable matrix).

2. Background

### Cell Based Therapies

In general, there are two major options in using cell-based therapies to manage and treat chronic and acute tissue damage in which the overall objective is the functional and/or cosmetic restoration of damaged tissue. These cell based therapy options include: 1) Cell Replacement—Use of cells to replace damaged tissue by establishing long-term engraftment; and 2) Supply Trophic Factors—Use of cells and compositions produced by cells (e.g., growth factors) to stimulate endogenous repair mechanisms through release of factors delivered or produced by cells without long-term engraftment.

Cell-based therapeutic options in managing and treating tissue damage also present the possibility for use of autologous or allogeneic cells. Each of these have certain advantages and disadvantages. Use of autologous cells involves the following factors or parameters:
Patient is the donor;
Requires manufacture of cell product on a patient-by-patient basis;
Variability in the identity, purity and potency of cell product; and,
Lag time between clinical decision to treat and availability of cells for transplant.

In contrast, the use of allogeneic cells involves the following factors or parameters:
Donor is second party (i.e., donor is not the patient);
Risk associated with donor variability;
Multiple patients treatable per manufactured batch of cell product;
Increased consistency of identity, purity and potency of cell product; and,
Decreased lag time between clinical decision to treat and availability of cell product.

### Organ and Tissue Repair

The regenerative potential of certain tissues in the mammalian body has been known for centuries, for example tissues like skin and bone are known to repair themselves after injury. However, a number of conditions and diseases of the central nervous system (i.e., brain and spinal cord), peripheral nervous system and heart adversely affect humans because of the deficit of regenerative capacity in the effected tissues. These conditions and diseases include, for example, spinal cord injury, amyotrophic lateral sclerosis (ALS), Parkinson's disease, stroke, Huntington's disease, traumatic brain injury, brain tumors, Fabry Disease, heart diseases (such as congestive heart failure and myocardial infarction). Clinical management strategies, for example, frequently focus on the prevention of further damage or injury rather than replacement or repair of the damaged tissue (e.g., neurons, glial cells, cardiac muscle); include treatment with exogenous steroids and synthetic, non-cellular pharmaceutical drugs; and have varying degrees of success which may depend on the continued administration of the steroid or synthetic drug.

For example, the majority of spinal cord injuries are compression injuries with the remaining cases involving complete transection of the spinal cord. Current therapeutic treatments for spinal cord injury include the prevention of additional spinal cord injury by physically stabilizing the spine through surgical and non-surgical procedures and by inhibiting the inflammatory response with steroidal therapy.

Additionally, aging is a major negative component to nearly every common disease affecting mammals, and one of the principle features of aging in a degeneration of many tissue including those of skin, bone, eye, brain, liver, kidney, heart, vasculature, muscle, et cetera. Furthermore, the already limited regenerative capacity of certain tissues of the body is known to decline with age, tissue maintenance and repair mechanisms in almost every tissue decline over the course of life.

Thus, there is a need to develop new, improved and effective methods of treatment for diseases and conditions, in particular, neurological and cardiac diseases and age-related degenerative conditions in humans.

### Erythropoiesis

Hematopoietic cells in a healthy human or other mammal do not ordinarily have a limited long-term self-renewal capability. However, the potential for catastrophic loss of blood (or need otherwise for a supplemental supply of blood) combined with limited supplies of donor blood, entails that methods for enhancing, maintaining, or generating red blood supplies in vitro are quite desirable.

Blood is a highly specialized circulating tissue consisting of several types of cells suspended in a fluid medium known as plasma. The cellular constituents are: red blood cells (erythrocytes), which carry respiratory gases and give it its red color because they contain hemoglobin (an iron-containing protein that binds oxygen in the lungs and transports it to tissues in the body), white blood cells (leukocytes), which tight disease, and platelets (thrombocytes), cell fragments which play an important part in the clotting of the blood. Medical terms related to blood often begin with hemo- or hemato- (BE: haemo- and haemato-) from the Greek word “haima” for “blood.” Blood cells are produced in the bone marrow; in a process called hematopoiesis. Blood cells are degraded by the spleen and liver. Healthy erythrocytes have a plasma half-life of 120 days before they are systematically replaced by new erythrocytes created by the process of hematopoiesis. Blood transfusion is the most common therapeutic use of blood. It is usually obtained from human donors. As there are different blood types, and transfusion of the incorrect blood may cause severe complications, cross-matching is done to ascertain the correct type is transfused.

A shortage of blood donors and inadequate supplies of red blood cells for transfusion is a common problem in treating patients worldwide. Accordingly, there is a need for new, improved and effective methods of increasing the availability of red blood cells as this would provide a means for alleviating at least some of the global shortages in red blood cell supplies.

### Skin

There are currently available a number of different treatments for wounds of the skin such as epidermal replacement products, dermal replacement products, artificial skin products, and wound dressings. Examples of some of these are described briefly below.

### Epidermal Replacement Products

According to the manufacturer, EPICEL™ (Genzyme Corp., Cambridge, Mass.) is composed of autologous epidermal cells skin grown from biopsy of patients own skin for treatment of burns. Cells are co-cultured with mouse feeder cell line into sheets of autologous epidermis.

According to the manufacturer, MYSKIN™ (CellTran LTD, Sheffield, S1 4DP United Kingdom) is a cultured autologous epidermal substitute for the treatment of burns, ulcers and other non-healing wounds. MYSKIN™ contains living cells expanded from the tissue of individual patients. MYSKIN™ comprises a layer of keratinocytes (epidermal cells) on an advanced polymer-like coating which facilitates the transfer of cells into the wound where they can initiate healing. MYSKIN™ uses a medical grade silicone substrate layer to support cell delivery, wound coverage and allow exudate management.

According to the manufacturer, EPIDEX™ (Modex Therapeutics Ltd, Lausanne, Switzerland) is an autologous epidermal skin equivalent that is grown directly from stem and pre-cursor cells derived from hair taken directly from a patient in a non-surgical procedure.

According to the manufacturer, CELLSPRAY™ (Clinical Cell Culture Europe Ltd, Cambridge CB2 1NL, United Kingdom) is a cultured epithelial autograft suspension that is sprayed onto injured skin in order to provide a rapid epidermal cover, promote healing and optimize scar quality.

### Dermal Replacement Products

According to the manufacturer, INTEGRA™ Dermal Regeneration Template (Integra LifeSciences Corporation, Plainsboro, N.J.) is a bilayer membrane system for skin replacement. The dermal replacement layer is made of a porous matrix of fibers of cross-linked bovine tendon collagen and a glycosaminoglycan (chondroitin-6-sulfate) that is manufactured with a controlled porosity and defined degradation rate. The temporary epidermal substitute layer is made of synthetic polysiloxane polymer (silicone) and functions to control moisture loss from the wound. The collagen dermal replacement layer serves as a matrix for the infiltration of fibroblasts, macrophages, lymphocytes, and capillaries derived from the wound bed.

According to the manufacturer, DERMAGRAFT™ (Advanced Biohealing Inc., La Jolla, Calif.) Allogeneic newborn fibroblasts grown on a biodegradable mesh scaffold, indicated for full-thickness diabetic ulcers.

According to the manufacturer, PERMACOL™ (Tissue Science Laboratories, Inc. Andover, Mass. 01810) Permacol™ surgical implant is collagen-derived from porcine dermis which, when implanted in the human body, is non-allergenic and long-lasting.

According to the manufacturer, TRANSCYTE™ (Advanced Biohealing Inc., La Jolla, Calif. 92037) TRANSCYTE™ is a human foreskin-derived fibroblast temporary skin substitute (allogeneic). The product consists of a polymer membrane and newborn human fibroblast cells cultured under aseptic conditions in vitro on a nylon mesh. Prior to cell growth, this nylon mesh is coated with porcine dermal collagen and bonded to a polymer membrane (silicone). This membrane provides a transparent synthetic epidermis when the product is applied to the burn. The human fibroblast-derived temporary skin substitute provides a temporary protective barrier. TRANSCYTE™ is transparent and allows direct visual monitoring of the wound bed.

According to the manufacturer, RENGRANEX™ Gel (Ortho-McNeil Pharmaceutical, Inc.© ETHICON, INC.) is a topical wound care product made of recombinant PDGF in a gel.

### Artificial Skin Products (Epidermal and Dermal Combination Products)

According to the manufacturer, PERMADERM™ (Cambrex Bio Science Walkersville, Inc., Walkersville, Md.) PERMADERM™ is constructed from autologous epidermal and dermal layers of the skin and is indicated for the treatment of severe burns. The product is reported to be pliable and to grow with the patient.

According to the manufacturer, ORCEL™ (Ortec International, New York, N.Y.) Bilayered construct made from allogeneic epidermal cells and fibroblasts cultured in bovine collagen, indicated for split-thickness burns. The manufacturer reports no evidence of product-derived DNA detectable in two human patients treated with product at 2 or 3 weeks, respectively.

According to the manufacturer, APLIGRAF™ (Smith & Nephew, London, WC2N 6LA United Kingdom) Allogeneic epidermal cells and fibroblasts cultured in bovine collagen, indicated for venous leg ulcers.

### Wound Dressings

According to the manufacturer, 3M™ TEGADERM™ Transparent Film Dressing (3M, St. Paul, Minn.) is a breathable film that provides a bacterial and viral barrier to outside contaminants.

According to the manufacturer, TISSEEL™ VH Fibrin Sealant (Baxter, Deerfield, Ill.) is indicated for use as an adjunct to hemostasis.

### SUMMARY OF THE INVENTION

The present invention relates to the production and use of stable cell populations and compositions produced thereby. The present invention relates primarily to treatments involving use of allogeneic cells. However, it would also be equally possible to perform these same treatments using autologous cells. The present invention also relates in part to treatment of dermotologic conditions, such as skin wounds and immunological disorders and diseases involving the skin.

The term “stable cell population” as used herein means an isolated, in vitro cultured, cell population that when introduced into a living mammalian organism (such as a mouse, rat, human, dog, cow, etc.) does not result in detectable production of cells which have differentiated into a specialized cell type or cell types (such as a chondrocyte, adipocyte, osteocyte, etc.) and wherein the cells in the cell population express, or maintain the ability to express or the ability to be induced to express, detectable levels of at least one therapeutically useful composition (such as membrane bound or soluble TNF-alpha receptor, IL-1R antagonists, IL-18 antagonists, compositions shown in Table 1A, 1B, 1C, etc.).

Another characteristic of the stable cell populations of the present invention is that the cells do not exhibit ectopic differentiation. The term “ectopic” means “in the wrong place” or “out of place”. The term “ectopic” comes from the Greek “ektopis” meaning “displacement” (“ek”, out of +“topos”, place=out of place). For example, an ectopic kidney, is one that is not in the usual location; or, an extrauterine pregnancy is an “ectopic pregnancy”. In the present context, an example of ectopic differentiation would be cells that when introduced into cardiac tissue, produce bone tissue-like calcifications and/or ossifications. This phenomenon has been demonstrated to occur, for example, when mesenchymal stem cells are injected into cardiac tissue. See, Breitbach et al., “Potential Risks of Bone Marrow Cell Transplantation Into Infarcted Hearts,” Blood, Vol. 110, No. 4 (August 2007).

The present invention relates to the generation and use of expanded, in vitro cultured, self-renewing colony forming somatic cells (hereinafter referred to as “CF-SC”), and products produced by such cells, for the treatment of a variety of diseases and disorders. Further, the present invention also relates to the generation and use of extensively expanded, in vitro cultured, self-renewing colony forming somatic cells (hereinafter referred to as “exCF-SC”), and products produced by such cells, for the treatment of a variety of diseases and disorders. ExCF-SC are self-renewing colony forming somatic cells (CF-SC) which have undergone at least about 30, at least about 40, or at least about 50 cell population doublings during in vitro cultivation. Hence, self-renewing colony forming somatic cells which have been expanded in vitro are hereinafter referred to as “CF-SC” (such that, unless specified otherwise, this term encompasses both cell populations which have undergone less than about 30 population doublings (e.g., less than about 5, less than about 10, less than about 15, less than about 20, less than about 25 population doublings) and also cell populations which have undergone more than about 30, more than about 40, or more than about 50 populations doublings in vitro). One particular example of CF-SC are adult human bone marrow-derived somatic cells (hereinafter referred to as “ABM-SC”). Further, one particular example of exCF-SC are adult human bone marrow-derived somatic cells which have undergone at least about 30, at least about 40, or at least about 50 cell population doublings during in vitro cultivation (hereinafter referred to as “exABM-SC”). Accordingly, the term “ABM-SC”, unless specified otherwise, encompasses both ABM-SC cell populations which have undergone less than about 30 population doublings (e.g., less than about 5, less than about 10, less than about 15, less than about 20, less than about 25 population doublings) and also ABM-SC cell populations which have undergone more than about 30, more than about 40, or more than about 50 populations doublings in vitro).

The term “extensively expanded” as used herein refers to cell populations which have undergone at least about 30 or more cell population doublings and wherein the cells are non-senescent, are not immortalized, and continue to maintain the normal karyotype found in the cell species of origin.

As used herein, the term “substantial capacity for self-renewal” means having the ability to go through numerous cycles of cell division resulting in the production of multiple generations of cell progeny (thus, with each cell division, one cell produces two “daughter cells” wherein at least one daughter cell is capable of further cell division). One measure of “substantial capacity for self-renewal” is indicated by the ability of a cell population to undergo at least about 10, 15, 20, 25, 30, 35, 40, 45, 50 or more cell doublings. Another measure of “substantial capacity for self-renewal” is indicated by maintenance of the ability of a cell population to re-populate, or approach confluence in, a tissue culture vessel after cell culture passaging (when the same or similar culture conditions are maintained). Thus, an example of “substantial capacity for self-renewal” is demonstrated when a cell population continues to re-populate a tissue culture vessel in a period of time of at least about 25%, 50%, 60%, 70%, 80%, 90%, 95% or 100% of the time required for such re-population during early cell culture doublings (such as before a cell population has undergone more than about 10 population doublings). Another measure of “substantial capacity for self-renewal” is maintenance of a consistent rate of population doubling time or of a consistent and relatively rapid rate of population doubling.

As used herein, the term “substantially no multipotent differentiation capacity” means cell populations which cannot differentiate into multiple different types of cells, either in vitro or in vivo. An example of cells which do have substantial multipotent differentiation capacity are hematopoietic stem cells which can differentiate into red blood cells, T-cells, B-cells, platelets, etc. either in vitro or in vivo. Another example of cells which do have substantial multipotent differentiation capacity are mesenchymal stem cells which can differentiate, for example, into osteocytes (bone), adipocytes (fat), or chondrocytes (cartilage). In contrast, cells in a cell population which have “substantially no multipotent differentiation capacity” cannot differentiate into multiple cell types in vitro or when introduced into an organism or target tissue(s) in vivo. In a preferred embodiment of the invention, a cell population with “substantially no multipotent differentiation capacity” is one in which at least about 80%, 90%, 95%, 98%, 99% or 100% of the cells in the cell population cannot be induced to detectably differentiate in vitro or in vivo into more than one cell type. A “unipotent” cell or “unipotent progenitor cell” is an example of a cell which has substantially no multipotent differentiation capacity.

As used herein, “stem cell” means a cell or cells possessing the following two properties: 1) capacity for self-renewal, which is the ability to go through numerous cycles of cell division while maintaining the undifferentiated state; and, 2) differentiation potency, which is the capacity to change into one or more kinds of mature cell types and, upon such change, no longer undergoing cycles of cell division (for example, capacity to change into an osteocyte, adipocyte, chondrocyte, etc.). As used herein, differentiation potency means the cells are either totipotent, pluripotent, multipotent, or unipotent progenitor cells. A “mesenchymal stem cell” is a stem cell of this same definition but wherein said cell has been derived or obtained from mesenchyme tissue (such as, for example, bone marrow, adipose or cartilage). See, Horwitz et al., “Clarification of the nomenclature for MSC: The International Society for Cellular Therapy position statement”, Cytotherapy, vol. 7, no. 5, pp. 393-395 (2005); and references cited therein.

As used herein, “totipotent” means cells which can become any type of cell as may be found during any stage of development in the organism of the cells origin. Totipotent cells are typically produced by the first few divisions of the fertilized egg (i.e., following fusion of an egg and sperm cell). Thus, totipotent cells can differentiate into embryonic and extraembryonic cell types.

As used herein, “pluripotent” means cells which can differentiate into cells derived from any of the three germ layers (endoderm, mesoderm, ectoderm) found in the organism of the cells origin.

As used herein, “multipotent” means cells which can produce multiple types (i.e., more than one type) of differentiated cells. A mesenchymal stem cell is an example of a multipotent cell.

As used herein, “unipotent” means cells which can produce only one cell type. Unipotent cells have the property of self-renewal, but can change into only one kind of mature cell type.

As used herein, “normal karyotype” means having a genetic composition comprising chromosomes of the number and of the structure typically found in, and considered normal for, the species from which the cells are derived.

As used herein, “connective tissue” is one of the four types of tissue usually referenced in traditional classifications (the others being epithelial, muscle, and nervous tissue). Connective tissue is involved in organism and organ structure and support and is usually derived from mesoderm. As used herein, “connective tissue” includes those tissues sometimes referred to as “connective tissue proper”, “specialized connective tissues”, and “embryonic connective tissue”.

“Connective tissue proper” includes areolar (or loose) connective tissue, which holds organs and epithelia in place and has a variety of proteinaceous fibres, including collagen and elastin. Connective tissue proper also includes dense connective tissue (or, fibrous connective tissue) which forms ligaments and tendons.

“Specialized connective tissue” includes blood, bone, cartilage, adipose and reticular connective tissue. Reticular connective tissue is a network of reticular fibres (fine collagen, type III) that form a soft skeleton to support the lymphoid organs (lymph nodes, bone marrow, and spleen)

“Embryonic connective tissue” includes mesenchymal connective tissue and mucous connective tissue. Mesenchyme (also known as embryonic connective tissue) is the mass of tissue that develops mainly from the mesoderm (the middle layer of the trilaminar germ disc) of an embryo. Viscous in consistency, mesenchyme contains collagen bundles and fibroblasts. Mesenchyme later differentiates into blood vessels, blood-related organs, and connective tissues. Mucous connective tissue (or mucous tissue) is a type of connective tissue found during fetal development; it is most easily found as a component of Wharton's jelly (a gelatinous substance within the umbilical cord which serves to protect and insulate cells in the umbilical cord).

As used herein, “immortalized” refers to a cell or cell line which can undergo an indefinite number of cell doublings in vitro. Immortalized cells acquire such ability through genetic changes which eliminate or circumvent the natural limit on a cells ability to continually divide. In contrast, “non-immortalized” cells are eukaryotic cells which, when taken directly from the organism and cultured in vitro (producing a “primary cell culture”), can undergo a limited number of cell doublings before senescencing (losing ability to divide) and dying. For example, primary cultures of most types of mammalian, non-immortalized cells can usually undergo a relatively defined but reproducibly limited range of cell doublings (depending on the primary cell type) before differentiating, senescing, or dying.

As used herein “long-term engraftment” means the detectable presence of donor cells residing within (or as part of) target tissue to which (or in which) said cells were delivered after more than about 4 weeks from the time of administration. “More than about 4 weeks” includes time periods of more than about 5, 6, 7, 8, 10, 12, 14, 16, 18, 20, and 24 weeks. “More than about 4 weeks” also includes time periods of more than about 6 months, 8 months, 10 months, 12 months, 18 months, 24 months, 30 months, 36 months, 42 months and 48 months.

The present invention also relates to manipulation of CF-SC and exCF-SC cell populations during cultivation to modulate (i.e., up- or down-regulate) production of various soluble or secreted compositions produced by the in vitro cultured and expanded self-renewing colony forming cells.

The present invention also relates to extensively expanded cell populations which are characterized by loss of ability to differentiate into bone cells (osteocytes). For example, the present invention relates to extensively expanded cell populations which are characterized by loss of ability to generate calcium deposits when cultured under osteoinductive conditions, including with or without cultivation in the presence of the supplemental bone morphogen Noggin (see Example 16). (Mouse and Human Noggin: See, e.g., the U.S. National Center for Biotechnology PubMed Protein Database Accession Nos. NP_{—}032737 and NP_{—}005441 (respectively); see also e.g., Valenzuela, et al., “Identification of mammalian noggin and its expression in the adult nervous system”, J. Neurosci. 15 (9), 6077-6084 (1995)).

The present invention also relates to extensively expanded cell populations characterized by the loss of ability to differentiate into bone cells and/or loss of ability to generate calcium deposits (as described above), but wherein said cell populations continue to secrete, or maintain the ability to secrete or to be induced to secrete, at least one therapeutically useful composition.

The present invention also relates to cell-based and tissue-engineering therapies; particularly, methods of using and/or administering CF-SC and exCF-SC, or compositions produced by such cells, including administration via incorporation in, or mixture with, pharmaceutically acceptable carriers (such as a pharmaceutically acceptable solution or a transient, permanent, or biodegradable matrix).

The present invention also relates to expanded (i.e., in vitro cultured and passaged) and extensively expanded cell populations which are preferably negative for expression of the STRO-1 cell surface marker. See, e.g., Stewart et al., “STRO-1, HOP-26 (CD63), CD49a and SB-10 (CD166) as markers of primitive human marrow stromal cells and their more differentiated progeny: a comparative investigation in vitro” Cell Tissue Res. 2003 September; 313(3):281-90; and, Dennis et al., “The STRO-1+ marrow cell population is multipotential” Cells Tissues Organs. 2002; 170(2-3):73-82; and, Oyajobi et al., “Isolation and characterization of human clonogenic osteoblast progenitors immunoselected from fetal bone marrow stroma using STRO-1 monoclonal antibody”, J Bone Miner Res. 1999 March; 14(3):351-61.

The present invention also relates to manufacture and use of pharmaceutically acceptable compositions containing CF-SC and exCF-SC (for example, ABM-SC and exABM-SC) with additional structural and/or therapeutic components. As one example, CF-SC or exCF-SC (for example, ABM-SC or exABM-SC) and collagen may be combined in a pharmaceutically acceptable solution to generate compositions in liquid, semi-solid, or solid-like forms (matrices) for use, for example, in the treatment, repair, and regeneration of skin disorders (e.g., skin wounds such as burns, abrasions, lacerations, ulcers, infections).]

The present invention relates generally to use of self-renewing cells, referred to herein as colony-forming somatic cells (CF-SC) including extensively passaged colony-forming somatic cells (exCF-SC). Examples of such cells are adult human bone marrow-derived somatic cells (ABM-SC) including extensively passaged adult human bone marrow-derived somatic cells (exABM-SC), for use in treatment of various diseases and disorders; particularly diseases and disorders involving ischemia, trauma, and/or inflammation (such as, for example, heart failure due to acute myocardial infarction (AMI) and stroke).

Self-renewing colony-forming somatic cells (CF-SC) such as adult human bone marrow-derived somatic cells (ABM-SC) as used in the present invention are prepared as described in U.S. Patent Publication No. 20030059414 (U.S. application Ser. No. 09/960,244, filed Sep. 21, 2001) and U.S. Patent Publication No. 20040058412 (U.S. application Ser. No. 10/251,685, filed Sep. 20, 2002). Each of these patent applications are hereby incorporated by reference in their entirety. In particular, CF-SC isolated from a source population of cells (such as, for example, from bone marrow, fat, skin, placenta, muscle, umbilical cord blood, or connective tissue) are cultured under low oxygen conditions (e.g., less than atmospheric) and passaged at low cell densities such that the CF-SC maintain an essentially constant population doubling rate through numerous population doublings. After expansion of the CF-SC to an appropriate cell number, the CF-SC may be used to generate the compositions of the present invention. For example, after expansion of the CF-SC in vitro for at least about 30, at least about 40, or at least about 50 cell population doublings exCF-SC may be used to generate compositions of the present invention. In one embodiment CF-SC and exCF-SC, as used in the present invention, are derived from bone marrow (and are referred to herein as ABM-SC and exABM-SC, respectively).

One embodiment of CF-SC and exCF-SC (such as for example, ABM-SC and exABM-SC, respectively), as used in the present invention, is an isolated cell population wherein the cells of the cell population co-express CD49c and CD90 and wherein the cell population maintains a doubling rate of less than about 30 hours after at least about 30, at least about 40, or at least about 50 cell population doublings.

Another embodiment of CF-SC and exCF-SC (such as for example, ABM-SC and exABM-SC, respectively), as used in the present invention, is an isolated cell population wherein the cells of the cell population co-express CD49c, CD90, and one or more cell surface proteins selected from the group consisting of CD44, HLA Class-1 antigen, and β (beta) 2-Microglobulin, and wherein the cell population maintains a doubling rate of less than about 30 hours after at least about 30, at least about 40, or at least about 50 cell population doublings.

Another embodiment of CF-SC and exCF-SC (such as for example, ABM-SC and exABM-SC, respectively), as used in the present invention, is an isolated cell population wherein the cells of the cell population co-express CD49c and CD90, but are negative for expression of cell surface protein CD10, and wherein the cell population maintains a doubling rate of less than about 30 hour after at least about 30, at least about 40, or at least about 50 cell population doublings.

Another embodiment of CF-SC and exCF-SC (such as for example, ABM-SC and exABM-SC, respectively), as used in the present invention, is an isolated cell population wherein the cells of the cell population co-express CD49c, CD90, and one or more cell surface proteins selected from the group consisting of CD44, HLA Class-1 antigen, and β (beta) 2-Microglobulin, but are negative for expression of cell surface protein CD10, and wherein the cell population maintains a doubling rate of less than about 30 hours after at least about 30, at least about 40, or at least about 50 cell population doublings.

Another embodiment of CF-SC and exCF-SC (such as for example, ABM-SC and exABM-SC, respectively), as used in the present invention, is an isolated cell population wherein the cells of the cell population express one or more proteins selected from the group consisting of soluble proteins shown in Table 1A, 1B and 1C, and wherein the cell population maintains a doubling rate of less than about 30 hours after at least about 30, at least about 40, or at least about 50 cell population doublings.

Damaged tissues and organs may result from, for example, disease (e.g., heritable (genetic) or infectious diseases (such as bacterial, viral, and fungal infections)), physical trauma (such as burns, lacerations, abrasions, compression or invasive tissue and organ injuries), ischemia, aging, toxic chemical exposure, ionizing radiation, and dysregulation of the immune system (e.g., autoimmune disorders).

The present invention encompasses the use of CF-SC and exCF-SC (such as for example, ABM-SC and exABM-SC, respectively), CF-SC and exCF-SC purified protein fractions, supernatants of CF-SC and exCF-SC conditioned media, and fractions of cell-supernatants derived from CF-SC and exCF-SC conditioned media. In one embodiment of the invention, the above mentioned components may be combined with, or introduced into, physiologically compatible biodegradable matrices which contain additional components such as collagen and/or fibrin (for example, purified natural or recombinant human, bovine, or porcine collagen or fibrin), and/or polyglycolic acid (PGA), and/or additional structural or therapeutic compounds. Combination matrices such as these may be administered to the site of tissue or organ damage to promote, enhance, and/or result in repair and/or regeneration of the damaged tissue or organ.

Embodiments of the invention include use of CF-SC and exCF-SC (such as for example, ABM-SC and exABM-SC, respectively), incorporated into pharmaceutically acceptable compositions which may be administered in a liquid, semi-solid, or solid-like state. Embodiments of the invention may be administered by methods routinely used by those skilled in the relevant art, such as for example, by topical application, as spray-on or aerosolized compositions, by injection, and implantation.

Use of CF-SC and exCF-SC (such as for example, ABM-SC and exABM-SC, respectively), cells and compositions produced by these cells as described in the present invention for tissue regenerative therapies may provide a number of benefits compared to previously described tissue regenerative therapies and products. For example, use of the CF-SC and exCF-SC (such as for example, ABM-SC and exABM-SC, respectively), exABM-SC cells and compositions produced thereby provides a means of tissue regenerative therapy which may exhibit reduced adverse immune responses (such as reduced inflammation and T-cell activation; see e.g., Examples 3A, 3B, 5, 18, and 19. Moreover, since ABM-SC and exABM-SCs are immunologically silent, subjects do not need to be HLA-matched or pre-conditioned prior to treatment. See, Example 10, Part II; see also, FIG. 17.

The present invention also relates to the use of CF-SC and exCF-SC (such as expanded and extensively expanded adult human bone marrow-derived somatic cells (human ABM-SC and exABM-SC, respectively)), and the cell products generated by these cells, for inducing, enhancing, and/or maintaining hematopoiesis (in particular, for the in vitro generation and production of red blood cells (erythrocytes) from hematopoietic progenitor cells in a process called erythropoiesis). Thus, another embodiment of the invention encompasses the use of such cells and/or compositions produced by such cells, to induce, enhance, and/or maintain the generation and production of red blood cells (erythrocytes).

Another example of the field of the invention relates to the prevention and treatment of immune, autoimmune, and inflammatory disorders via use of such cells, cell populations, and compositions produced thereby.

In another example, the present invention provides compositions and methods for repair and regeneration of wounds of the skin (i.e., epidermis, dermis, hypodermis); including the manufacture and use of liquid, semi-solid, and solid-like matrices which incorporate CF-SC and exCF-SC (for example, human ABM-SC and exABM-SC), or products generated by such cells, and additional structural or therapeutic compounds.

### Exemplary Results of Preclinical Studies

In vivo preclinical pharmacology studies have demonstrated the beneficial effects of ABM-SC in treating myocardial infarction and stroke. For example, in a study investigating the effects of intra-cardiac injection of hABM-SC in a rat model of myocardial infarction (in particular, to determine the efficacy of hABM-SC in restoring cardiac function post-AMI (acute myocardial infarction) and evaluate distribution and disposition of hABM-SC), it was shown that hABM-SC produced a significant improvement in cardiac function and significantly reduced fibrosis. Furthermore, the hABM-SC were not observed to remain in the heart four weeks after cardiac injection, nor in any of the peripheral organs examined eight weeks after injection. Additionally, in a study investigating the safety and efficacy of porcine and human ABM-SC in an AMI model in pigs (in particular, to evaluate the feasibility, safety and efficacy of percutaneous, NOGA™-guided endomyocardial administration of cells through a MYOSTAR™ catheter) it was demonstrated that this particular delivery method was well-tolerated and led to significant improvements in cardiac parameters. Likewise, in a comparison of the method of delivery of hABM-SC and stroke recovery (in particular, to determine efficacy of hABM-SC in promoting neuromotor recovery from ischemic stroke) it was observed that I.V. or intra-cerebral treatment resulted in significant improvements in neuromotor activity.

### DESCRIPTION OF THE DRAWINGS

FIG. 1 shows a 2-dimensional SDS PAGE separation (pH 3.5 to 10; 12% polyacrylamide) of proteins secreted by human adult bone marrow-derived somatic cells (ABM-SC at about 27 population doublings). Each spot on the gel represents a separate and distinct protein, ranging in size from approximately 5-200 kilodaltons (kDa). The X-axis shows proteins separated according to isoelectric point (pH 3.5 to 10). The Y-axis shows proteins separated according to molecular weight (via passage through 12% polyacrylamide).

FIG. 2 shows photomicrographs of PC-12 differentiation into neurons using nerve growth factor (NGF) and conditioned media derived from human exABM-SC (at about 43 population doublings). A) RPMI-ITS medium only. B) RPMI-ITS supplemented with NGF. C) RPMI-ITS supplemented with a 1:50 dilution of concentrated control media and NGF. D) RPMI-ITS supplemented with a 1:50 dilution of concentrated conditioned media derived from human ABM-SC and NGF. Arrows indicate neurite outgrowth. Extent of neurite outgrowth in panel D is significantly more robust than that of panel B and C.

FIG. 3 is a graphical representation of inhibition of mitogen-induced T cell proliferation using human ABM-SC. Lot # RECB801 represents ABM-SC that have been sub-cultured to about 19 population doublings and Lot # RECB906 represents exABM-SC which have been sub-cultured to about 43 population doublings. To stimulate T cell proliferation, cultures were inoculated with 2.5 or 10 micrograms/mL Phytohaemagglutinin (“PHA”). Cells were then harvested after 72 hrs later and stained with CD3-PC7 antibody. Human mesenchymal stem cells were used as a positive control (Cambrex). (Human mesenchymal stem cells were obtained from Cambrex Research Bioproducts; now owned by Lonza Group Ltd., Basel, Switzerland).

FIG. 4 shows photomicrographs of pig skin 7 days after surgically-induced incisional wounding. A) Wound No. 3 treated with allogeneic porcine ABM-SC (at about 28 population doublings) shows complete wound closure with virtually no scar. B) In comparison, Wound No. 4 treated with vehicle only reveals a visible scar. C) The graph represents histomorphometric scoring of tissue sections from both treatment groups and shows a statistically significant reduction (p=0.03) in the number of histiocytes in the porcine ABM-SC treated wounds (statistical significance determined using a two-tailed unpaired T-test); compare, bars for “Histiocytes” PBSG versus pABM-SC treated.

FIG. 5 is a graphical representation (top panel) of the extent of re-epithelialization across the incisional wounds 7 days post-treatment. Wounds treated with porcine ABM-SC (at about 28 population doublings) had a thicker epidermis than those treated with vehicle only. The photomicrograph in the lower left panel (B) shows (histologically) complete and anatomically correct repair of the epidermis in the wounds treated with porcine ABM-SC. The photomicrograph in the lower right panel (C) shows (histologically) porcine ABM-SC (arrow heads) which appear engrafted, at least transiently, in the hypodermis at this 7 day time point.

FIG. 6 is a graphical representation of ABM-SC mediated contraction of hydrated collagen gel lattices seeded 24 hours after cell reconstitution. Human ABM-SC (at about 27 population doublings) were reconstituted in liquid biodegradable collagen-based media (at 1.8×10⁶ cells/mL) and then stored for 24 hours at approximately 4-8° C. The following day the liquid cell suspension was placed into a culture dish to form a semi-solid collagen lattice. The semi-solid collagen lattices were maintained in a cell culture incubator to facilitate contraction over the course of three days. Collagen lattices prepared without cells did not contract, demonstrating that contraction is dependent upon the presence of cells.

FIG. 7 is a graphical representation of ABM-SC mediated contraction of hydrated collagen gel lattices seeded at different cell concentrations utilizing exABM-SC at about 43 population doublings. The data demonstrate that rate and absolute magnitude of contraction is related to cell number. Heat inactivated cells do not contract the gels, demonstrating that this activity is a biophysical event.

FIG. 8 is a graphical representation of ABM-SC mediated secretion of several cytokines and matrix proteases (i.e., IL-6, VEGF, Activin-A, MMP-1, and MMP-2) when cultured for 3 days in hydrated collagen gel lattices utilizing exABM-SC at about 43 population doublings.

FIG. 9 shows photomicrographs of human ABM-SC reconstituted in biodegradable collagen-based media as a liquid (left panel, A) or a semi-solid (right panel, B) (utilizing exABM-SC at about 43 population doublings). When reconstituted using this formulation, the cell suspension can remain as a liquid at 4° C. for more than 24 hrs. When placed in a culture dish and incubated at 37° C., the cell suspension will solidify within 1-2 hours, giving rise to a semi-solid structure than can be physically manipulated.

FIG. 10 shows photomicrographs of a solid-like neotissue formed by culturing human ABM-SC (at about 43 population doublings) reconstituted in the biodegradable collagen-based media for three days. The upper left panel (A) shows the pliability of the tissue when stretched. The upper right panel (B) shows the general texture of the solid-like neotissue. The lower panel (C) shows a histological section of the tissue stained by Masson's Trichrome, demonstrating the rich extracellular matrix synthesized by the ABM-SC. Control gels constructed by the same method, but lacking cells, do not stain blue by this method, demonstrating that the collagen and glycosaminoglycan-rich matrix is produced by the cells.

FIG. 11 shows an example of the quantities of multiple pro-regenerative cytokines secreted by human ABM-SC with and without TNF-alpha stimulation. When sub-cultured, ABM-SC secrete potentially therapeutic concentrations of several growth factors and cytokines known to augment angiogenesis, modulate inflammation and promote wound healing. ABM-SC have been shown to consistently secrete several cytokines and growth factors in vitro; including proangiogenic factors (e.g., SDF-1 alpha, VEGF, ENA-78 and angiogenin), immunomodulators (e.g., IL-6 and IL-8) and scar inhibitors/wound healing modulators (e.g., MMP-1, MMP-2, MMP-13 and Activin-A). Furthermore, the release of several of these factors is modulated by tumor necrosis factor alpha (TNF-alpha), a known inflammatory cytokine released during the course of acute tissue injury.

FIG. 12 shows a model injury-response cascade (inflammation, regeneration, and fibrosis from injury through scar) and examples of molecules that can play roles in inflammation, regeneration, and fibrosis.

FIG. 13 shows an example of improved cardiac function results in rats treated with human ABM-SC. Four weeks after treatment, rats receiving ABM-SC demonstrated significantly higher +dp/dt (peak positive rate of pressure change) values (A). Expressing changes in cardiac function over the course of the study by subtracting 0 week +dp/dt values from 4 week values (“delta +dp/dt”) demonstrated that while vehicle treated rats had decreases in cardiac function over the course of the study (negative delta), animals treated with either cell preparation showed significant improvement in cardiac function (B). Compared to vehicle treated rats, those receiving ABM-SC demonstrated significantly lower tau values (C), suggesting increased left ventricular compliance. Tau is the time constant of isovolumetric left ventricular pressure decay. For peak negative rate of pressure change (−dp/dt), expressing changes in cardiac function over the course of the study by subtracting 0 week −dp/dt values from 4 week values (“delta −dp/dt”) demonstrated that while vehicle-treated rats had decreases in cardiac function over the course of the study (negative delta), animals treated with cell preparation showed significant improvement in cardiac function (D). [*p<0.05, **p<0.01 by ANOVA]

FIG. 14 shows reduction of fibrosis and enhanced angiogenesis in a rat model myocardial infarct treated with human AMB-SC (hABM-SC). Semi-quantitative scoring was used to evaluate changes in infarct size in the hearts of rats receiving vehicle or ABM-SC seven days after myocardial infarction. Histopathological analysis, performed approximately 30 days after administration of ABM-SC, indicated significant reduction in infarct size in rats receiving hABM-SC compared to vehicle. According to a preset scale, rats receiving hABM-SC had histological scores approximately two points lower than vehicle controls. This figure shows an example of typical infarct size reduction.

FIG. 15 shows results obtained from histological, performed approximately 30 days after administration of ABM-SC, measurement of changes in the heart structure of rats receiving vehicle or ABM-SC seven days after myocardial infarction.

FIG. 16 shows that allogeneic human ABM-SC (RECB801) and exABM-SC (RECB906) suppress mitogen-induced T-cell proliferation in one-way MLR (mixed lymphocyte reaction) assay.

FIG. 17 shows that allogeneic porcine ABM-SC fail to illicit T-cell mediated immune response in a 2-way MLR challenge experiment. A Division Index was calculated for samples collected at baseline and 3 or 30 days post-treatment and then challenged in vitro with media, vehicle, pABM-SC or ConA. The average division index from all animals at Day 3 or Day 30 for PBMC cells which were stimulated with ConA was significantly higher than the division index for PBMC cells from vehicle and pABM-SC treated animals at both pre-treatment and necropsy (* p<0.05).

FIG. 18 shows the changes in cardiac fixed perfusion deficit size in three patients by comparison of baseline (BL) measurements, with measurements obtained 90 days post-treatment with hABM-SC.

FIG. 19 shows the changes in cardiac ejection fractions measured in three patients by comparison of baseline (BL) measurements with measurements obtained 90 days post-treatment with hABM-SC.

FIG. 20 shows examples of quantities of erythropoietic cytokines secreted in vitro by hABM-SC (i.e., IL-6, Activin-A, VEGF, LIF, IGF-II, SDF-1 and SCF). ABM-SC lots were tested for cytokine secretion using RAYBIO™ Human Cytokine Antibody Array (RayBiotech, Inc.). Cells were first cultured in serum-free Advanced DMEM (GIBCO™) for three days to generate conditioned medium (CM). The CM was then concentrated using CENTRICON™ PLUS-20 Centrifugal Filter Units (Millipore) prior to analysis.

FIG. 21 demonstrates that exABM-SC reduce TNF-α levels in vitro in a dose-dependent manner. Human exABM-SC (at about 43 population doublings) were tested for their ability to reduce TNF-α levels when cultured at various seeding densities (e.g. 10,000 cells/cm², 20.000 cells/cm², and 40,000 cells/cm²). Cells were cultured for 3 days in serum-free Advanced DMEM (GIBCO™) either alone or supplemented with 10 ng/mL TNF-α. Heat inactivated cells were also included as a negative control. Concentration of TNF is shown on the Y-axis. (Y-axis represents concentration of substances in media which has been concentrated 100×).

FIGS. 22A and 22B demonstrates that reduction of TNF-α appears to be mediated by the secretion of sTNF-RI and sTNF-RII by exABM-SC (at about 43 population doublings). Basal level expression of sTNF-RI occurs in the absence of a pro-inflammatory inducer (A), while sTNF-RII is detected at appreciable levels only when first primed with TNF-α (B). These data reveal an inverse relationship between the number of cells seeded and the levels of both sTNF-RI and sTNF-RII detected, suggesting that the secreted receptors themselves may be binding to and masking the TNF-α. (Y-axis represents concentration of substances in media which has been concentrated 100×).

FIG. 23 demonstrates that secretion levels of IL-IRA (by exABM-SC at about 43 population doublings) is dose-dependent. Basal level expression of IL-IRA occurs in the absence of a pro-inflammatory inducer, but when primed when TNF-α, soluble levels increase approximately O-fold. (Y-axis represents concentration of substances in media which has been concentrated 100×).

FIG. 24 shows expression of IL-1 receptor antagonist (IL-IRA) and IL-18 binding protein (IL-18BP) by exABM-SC. Human exABM-SC express basal levels of IL-1 receptor antagonist (IL-1RA; FIG. 24A) and IL-18 binding protein (IL-18BP; FIG. 24B) even in the absence of an inflammatory signal such as TNF-alpha.

FIGS. 25A, B, and C show that human ABM-SC reduce levels of TNF-alpha (FIG. 25A) and IL-13 (FIG. 25B) while simultaneously inducing elevated expression of IL-2 (FIG. 25C) in a Mixed PBMC reaction assay. (R=Responder PBMC, Self=Mitomycin-C treated PBMC isolated from same donor as Responder, Stim=Mitomycin-C treated PBMC isolated from a different donor.)

FIG. 26 shows a graphical representation of inhibition of mitogen-induced human peripheral blood mononuclear cell (PBMC) proliferation using human ABM-SC. RECB801 represents a particular lot of ABM-SC that have been sub-cultured to about 19 population doublings and # RECB906 represents a particular lot of ABM-SC that have been sub-cultured to about 43 population doublings. To stimulate PBMC proliferation, cultures were inoculated with 2.5 microg/mL phytohaemagglutinin. After 56 hours in culture, cells were pulsed with Thymidine-[Methyl-3H] and at 72 hours isotope incorporation was quantitated (CPM). Human mesenchymal stem cells (Cambrex) were included as a positive control.

FIG. 27 depicts the results of a medical-grade porcine-collagen gel contraction assay; demonstrating an effective dose response curve of collagen gel contraction as a function of increasing human exCF-SC density and increasing collagen gel concentration.

FIG. 28 depicts quantities of VEGF (Vascular Endothelial Growth Factor) produced within cultured human exCF-SC encapsulated in porcine-collagen gel neotissue; demonstrating increased VEGF concentrations within gels as a function of increasing cell density.

FIG. 29 depicts results obtained in an in vitro wound closure assay when conditioned media containing factors produced by human exCF-SC are compared to results obtained with non-conditioned media; demonstrating that conditioned media significantly increased the rate and magnitude of wound closure compared to non-conditioned media.

FIG. 30 depicts a quantitative determination of secreted factors present in conditioned media following exposure of human exCF-SC to IL-1 alpha (IL-1a) (10 ng/mL) for 24 hours; demonstrating that IL-1a induces expression of some factors and upregulates expression of others.

FIG. 31 depicts a quantitative determination of secreted factors present in conditioned media following exposure of human exCF-SC to tumor necrosis factor alpha (TNFa) (10 ng/mL) for 24 hours; demonstrating that TNFa induces expression of some factors and upregulates expression of others.

FIG. 32 depicts a quantitative determination of secreted factors present in conditioned media following exposure of human exCF-SC to interferon gamma (IFNg) (10 ng/mL) for 24 hours; demonstrating that IFNg induces expression of some factors and upregulates expression of others.

FIG. 33 summarizes effects of inflammatory factors on human exCF-SC secretion profile. A numeric code is used to indicate the degree and nature of these effects; divided into 5 categories based on the magnitude and direction of effect.

Code:

−2=reduction by >2;

−1=reduction by 0 to −2;

0=no change;

+1=induction <10;

+10=induction 10 to 1000;

+1000=induction >1000.

These results demonstrate that human exCF-SC modify their secretion profile in response to different inflammatory markers and therefore one might expect human ABMSC to have distinct effects depending upon the in vivo environment.

FIG. 34 solid boxes indicate (for example but without limitation) various biological systems upon which induction of the indicated factors may be useful in rendering therapeutic effects (e.g., vascular, immune, regenerative, inflammatory, and wound repair systems and mechanisms).

FIG. 35 demonstrates that nearly 200 transcripts are differentially expressed at least at least two fold (p≦0.01) in Neonatal Human Dermal Fibroblasts (NHDF) grown at 4% oxygen and seeded at 30 cells/cm² compared to NHDF grown at 20% oxygen and seeded at 3000 cells/cm². See also, Table 2.

FIG. 36 shows that adult bone marrow-derived cells from equine (horse) sources are capable of rapid proliferation and high numbers of cell doublings when cultured and passaged in vitro under conditions of low oxygen (4% oxygen) and low cell seeding densities (60 cells/cm²).

FIG. 37 demonstrates that adult bone marrow-derived equine (horse, EQ104) cell populations exhibit bioactivity (gel contraction) when cultured in a collagen matrix.

FIG. 38 depicts VEGF levels within cultured human exCF-SC seeded in Poly-Lactic-co-Glycolic Acid (PLGA) scaffolds; demonstrating an increase in VEGF contained within the PLGA constructs as a function of increasing cell density.

FIG. 39 shows photographs of various forms of bio-engineered constructs of the invention: A & B) human exCF-SC seeded porcine collagen gel after culture and cross-linking to generate a non-living mechanically stable bioactive construct; C) human exCF-SC seeded porcine collagen gel after culture and dehydration to generate a non-living thin film bioactive construct; and D) non-woven PLGA scaffold (left-lower corner) and human exCF-SC seeded non-woven PLGA scaffold cultured construct (right lower-corner. U.S. Quarter shown for size-comparison (center, top).

FIG. 40 is a flow chart illustrating a process of the invention for creating collagen-based, bioactive devices. In embodiments, the process involves 4 major steps as outlined.

FIG. 41 Fluorescent microscopic images of live/dead viability stained hABM-SC seeded collagen constructs.

FIG. 42 Collagen gel contraction measurements of hABM-SC seeded porcine collagen constructs over time.

FIG. 43 Quantification of VEGF by ELISA from hABM-SC seeded porcine collagen gel constructs within collected conditioned media and construct lysates.

FIG. 44 Cell viability in hABM-SC seeded collagen constructs without and with addition of 20 mM HEPES to culture media.

FIG. 45 Quantification of VEGF by ELISA within lysates of cultured hABM-SC seeded porcine collagen gel constructs.

FIG. 46 Collagenase digestion times and cell viability within digests of glutaraldehyde cross-linked cultured hABM-SC collagen constructs.

FIG. 47 Quantification of VEGF by ELISA within processed hABM-SC collagen constructs.

FIG. 48 hABM-SC viability after plating of minced collagen construct with processing.

FIG. 49 Quantification of VEGF by ELISA within lysates of varying device iterations.

FIG. 50 Quantification of VEGF by ELISA within lysates of varying device iterations.

FIG. 51 Quantification of VEGF by ELISA within lysates of varying device iterations.

FIG. 52 Photograph of glutaraldehyde cross-linked cultured collagen cell seeded construct prior to dehydration.

FIG. 53 Photograph of embodiments of devices during dehydration step.

FIG. 54 Photograph of 6601 devices seeded with cells.

FIG. 55 Photograph of the exemplary devices of the invention.

FIG. 56 Photograph of 6601 device iteration during suture retention testing.

FIG. 57 Quantification of VEGF by ELISA within lysates of varying device iterations.

FIG. 58 Quantification of VEGF by ELISA within lysates of device iterations.

FIG. 59 GBT Collagen-based, bioactive device prototypes. A. 46000, non-cross-linked. B. 46001, cross-linked with 0.001% gluteraldehyde. Image for 46000-001 not available.

FIG. 60 Repair of surgically-induced flexor tendon lesion using Kessler-Kajima suture (arrow head) and GBT device 46001.

FIG. 61 46001 cut down into a strip approximately 0.7-0.9 cm wide×2.8 cm in diameter, and wrapped around the digital flexor tendon.

FIG. 62 SCAFTEX PLGA 90/10 scaffold alone (lower left, SCAFTEX seeded with Garnet cell-therapy product, GBT009 (lower right). Quarter included for scale.

FIG. 63 GBT PLGA-based device implanted into the thumb after CMC arthroplasty.

### DETAILED DESCRIPTION OF THE INVENTION

The present invention relates to use of cell based therapies without relying on long-term cell engraftment. In particular, the invention relates to use of cells, and compositions produced by cells, in the treatment of various diseases and disorders; particularly those involving tissues and organs with limited self-renewal capability (such as, for example, neurological and cardiac tissues and organs). In embodiments of the invention, the cells of the invention are contacted with a patient in need of treatment. The term “patient” encompasses both human and non-human animals.

Typically, a stem cell or other early-stage progenitor cells lose plasticity because the cells have committed to a particular differentiation pathway. At the biomolecular level, as this process begins to occur the cell loses the ability to respond to certain signaling molecules (e.g., mitogens and morphogens) which would otherwise drive the cell to divide or become another cell type. Thus, as a cell begins to differentiate, it leaves the cell cycle (i.e., can no longer go through mitosis) and enters an irreversible state called GO wherein the cell can no longer divide. Entry into GO is also associated with replicative senescence (hallmarks of which include increased expression of intracellular proteins p21 and p53). Thus, loss of plasticity (the ability to differentiate into a variety of cell types) is typically considered a prelude to cellular differentiation or cellular senescence. Furthermore, loss of plasticity is also typically associated with the loss of a cells capacity for continued self-renewal. In contrast, to this typical and traditionally accepted scenario, an unexpected and surprising result of the present invention is that the exCF-SC of the present invention (e.g., exABM-SC) continue to self-renew (including self-renewal at a relatively constant rate) despite loss of plasticity. Accordingly, one embodiment of the present invention are therapeutically useful “end-stage cells” with a continued capacity for self-renewal (e.g., cells capable of continued self-renewal and production of trophic support factors (or “trophic support cells”)). In another embodiment, the exCF-SC and exABM-SC of the present invention do not express significant quantities of p21 and/or p53, wherein a “significant quantity” of said molecules is a quantity which is indicative of cell senescence (wherein senescence may require sufficient expression levels of p21, p53, and/or other cell cycle regulators).

Additionally, most experts in the field of the present invention would expect a non-hematopoietic stromal-type cell that has lost plasticity to have limited utility or capability of generating or promoting regeneration of organs and tissues. Thus, another surprising and unexpected result of the present invention, is the ability to generate extensively passaged CF-SC (e.g., ABM-SC) which have lost plasticity yet retain the ability to generate new tissue in vitro and to promote regeneration of tissue in vivo.

The present invention is drawn, inter alia, to methods of repairing, regenerating, and/or rejuvenating tissues using self-renewing cells, referred to herein as colony-forming somatic cells (CF-SC) (an example of which are adult human bone marrow-derived somatic cells (ABM-SC)). Self-renewing colony-forming somatic cells (CF-SC) such as adult human bone marrow-derived somatic cells (ABM-SC) as used in the present invention are prepared as described in U.S. Patent Publication No. 20030059414 (U.S. application Ser. No. 09/260,244, filed Sep. 21, 2001) and U.S. Patent Publication No. 20040058412 (U.S. application Ser. No. 10/251,685, filed Sep. 20, 2002). Each of these patent applications are hereby incorporated by reference in their entirety. Also incorporated by reference herein are U.S. Provisional Patent Applications 60/929,151 and 60/929,152 (each filed on Jun. 15, 2007), U.S. Provisional Patent Application 60/955,204 (filed on Aug. 10, 2007), and U.S. Provisional Patent Application 60/996,093 (filed on Nov. 1, 2007).

The invention also relates to compositions and matrices comprising conditioned cell culture derived from CF-SC cells. The invention further provides methods of treating medical conditions in a patient using conditioned cell culture derived from CF-SC cells. The term “conditioned cell culture derived from CF-SC cells” refers to the media that the CF-SC cells grew in, after the cells have been removed from the media. In embodiments, such conditioned cell culture derived from CF-SC cells is substantially free of the CF-SC cells. “Substantially free” means that all the cells have been removed or a majority of the cells have been removed. Optionally, the conditioned cell culture derived from CF-SC cells has been treated with pharmaceutical compounds, for example stimulatory factors such as Interleukin-1 beta (IL-1b), Interleukin-1 alpha (IL-1a), tumor necrosis factor alpha (TNF-a), interferon gamma (IFN-g), Interleukin-2 (IL-2), Transforming growth factor beta (TGF-b), Nerve growth factor (NGF), Epidermal growth factor (EGF), concavalin A (Con-A), and/or phytohemagglutinin (PHA), to name a few, to induce the production of conditioned cell culture media.

In particular, CF-SC isolated from a source population of cells (such as, for example, from bone marrow (ABM-SC and exABM-SC), fat, skin, placenta, muscle, umbilical cord blood, or connective tissue), are permitted to adhere to a cell culture surface in the presence of an appropriate media (such as for example, but not limited to, Minimal Essential Medium-Alpha (e.g., available from HYCLONE™) supplemented with 4 mM glutamine and 10% fetal bovine serum) and cultured under low oxygen conditions (such as for example, but not limited to, O₂ at about 2-5%, CO₂ at about 5%, balanced with nitrogen) and subsequently passaged at low cell densities (such as at about 30-1000 cells/cm²) such that the CF-SC maintain an essentially constant population doubling rate (such as for example, but not limited to, a doubling rate of less than about 30 hours) through numerous population doublings (such as for example, but not limited to, going through 10, 15, 20, 25, 30, 35, 40, 45 and/or 50 population doublings).

Embodiments of the invention may be generated with CF-SC and exCF-SC (for example, ABM-SC and exABM-SC) cultured under low oxygen conditions wherein said 07 concentrations range from about 1-20% (for example, wherein the O₂ concentration is about 1%, 2%, 3%, 4%, 5%, 6%, 7%, 8%, 9%, 10%, 11%, 12%, 13%, 14%, 15%, or 20%), plus CO₂ and balanced with nitrogen. For example, ABM-SC may be cultured under low oxygen conditions wherein said O₂ concentrations are about 20%, less than about 20%, about 15%, less than about 15%, about 10%, less than about 10%, about 7%, less than about 7%, about 6%, less than about 6%, about 5%, less than about 5%, about 4%, less than about 4%, about 3%, less than about 3%, about 2%, less than about 2%, about 1%; or, wherein said low oxygen conditions are in a range from about 1% to about 20%, about 1% to about 15%, about 1% to about 10%, about 1% to about 5%, about 5% to about 20%, about 5% to about 15%, about 5% to about 10%, about 10% to about 15%, about 10% to about 20%, about 2% to about 8%, about 2% to about 7%, about 2% to about 6%, about 2% to about 5%, about 2% to about 4%, about 2% to about 3%, about 3% to about 8%, about 3% to about 7%, about 3% to about 6%, about 3% to about 5%, about 3% to about 4%, about 4% to about 8%, about 4% to about 7%, about 4% to about 6%, about 4% to about 5%, about 5% to about 8%, about 5% to about 7%, about 5% to about 6%, or about 5%.

Embodiments of the invention may be generated with CF-SC and exCF-SC (for example, ABM-SC and exABM-SC) cultured under low oxygen conditions wherein CO₂ concentration range from about 1-15% (for example, wherein the CO₂ concentration is about 1%, 2%, 3%, 4%, 5%, 6%, 7%, 8%, 9%, 10%, 11%, 12%, 13%, 14%, or 15%), plus low O₂ and balanced with nitrogen. Embodiments of the invention may be generated with CF-SC and exCF-SC (for example, ABM-SC and exABM-SC) passaged by seeding cells at low cell densities, wherein said cell density ranges from about 1-2500 cells/cm² (for example, wherein the cell density is about 1, 2, 5, 10, 20, 30, 40, 50, 60, 70, 80, 90, 100, 200, 300, 400, 500, 600, 700, 800, 900, 1000, 1500, 2000 or 2500 cells/cm²). For example, ABM-SC may be passaged at seeding densities of less than about 2500 cell/cm², less than about 1000 cells/cm², less than about 500 cells/cm², less than about 100 cells/cm², less than about 50 cells/cm², less than about 30 cells/cm², or less than about 10 cells/cm². Embodiments of the invention may be generated with CF-SC and exCF-SC (for example, ABM-SC and exABM-SC) wherein the cell population doubling rates are maintained in a range of less than about 24-96 hours (for example, wherein the cell population doubling rate is maintained at less than about 24, 30, 36, 42, 48, 54, 60, 66, 72, 78, 84, 90, or 96 hours). Embodiments of the invention may be generated with CF-SC and exCF-SC (for example, ABM-SC and exABM-SC) wherein the cell population maintains an essentially constant doubling rate through a range of population doublings such as in a range of about 5-50 population doublings (for example, wherein the population doubling rate is maintained for about 5-10, 5-15, 5-20, 5-25, 5-30, 5-35, 5-40, 5-45, or 5-50 population doublings).

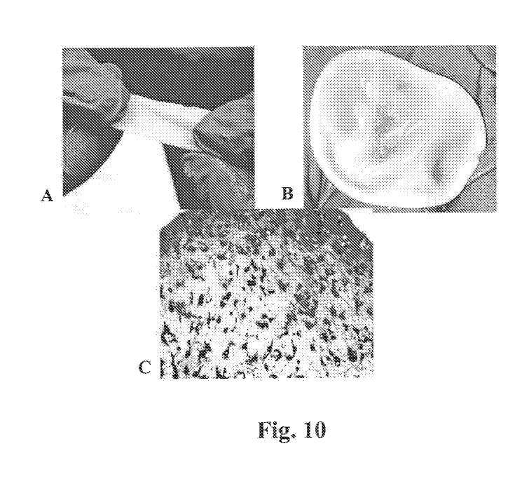

Embodiments of the invention include use of CF-SC and exCF-SC (for example, ABM-SC and exABM-SC) incorporated into pharmaceutically acceptable compositions which may be in a liquid, semi-solid, or solid-like state. Use of the terms “liquid, semi-solid, or solid-like state” is intended to indicate that the pharmaceutically acceptable composition in which the cells are contained can span a range of physical states from 1) a common liquid state (such as in an ordinary physiological saline solution); 2) to a wide-range of low-to-highly viscous states including jelly-like, gelatinous, or viscoelastic states (wherein the pharmaceutical composition contains from very high to very low levels of extracellular water, for example, such that the composition ranges in viscosity from a state where it “oozes” slowly like oil or honey to increasingly gelatinous or viscoelastic states which may be jelly-like, pliable, semi-elastic and/or malleable; 3) to a solid-like state (having very low levels of extracellular water) wherein the living cells within the matrix have remodeled the milieu in which they were initially suspended into a durable, non-gelatinous, but still pliable, semi-elastic, and malleable matrix (which, for example, has some of the same pliable, semi-elastic properties of mammalian skin); see, FIGS. 10A and 10B.

Viscoelasticity, also known as anelasticity, describes materials that exhibit both viscous and elastic characteristics when undergoing plastic deformation. Viscous materials, like honey, resist shear flow and strain linearly with time when a stress is applied. Elastic materials strain instantaneously when stretched and just as quickly return to their original state once the stress is removed. Viscoelastic materials have elements of both of these properties and, as such, exhibit time dependent strain.

Clinical administration of cells in liquid, semi-solid, and solid-like vehicles will enable application of treatments that shape to the contour of the wound bed, without trapping unwanted exudate in the wound.

Combining soluble matrix components such as collagens or fibrin with CF-SC and exCF-SC (for example, ABM-SC and exABM-SC) induces the cell population to up-regulate expression of important secreted proteins such as cytokines and matrix metalloproteinases. Moreover, application of ABM-SC to surgically-induced wounds appears to facilitate wound closure and prevent scarring thereby resulting in minimal scarring (see, e.g., Example 7). In embodiments of the invention, the matrix comprising CF-SC and/or exCF-SC cells is contacted with a patient in need of treatment, e.g., a patient having a wound.

Additionally, the apparent immunomodulatory properties of CF-SC and exCF-SC (such as, ABM-SC and exABM-SC) (see, e.g., Example 5) make compositions and therapies incorporating these cells attractive for the treatment of immunological disorders and diseases involving the skin (dermotologic), such as for example, but not limited to, chronic inflammatory dermatoses, psoriasis, lichen planus, lupus erythematosus (LE), graft-versus-host disease (GVHD), and drug eruptions (i.e., adverse cutaneous drug reactions).

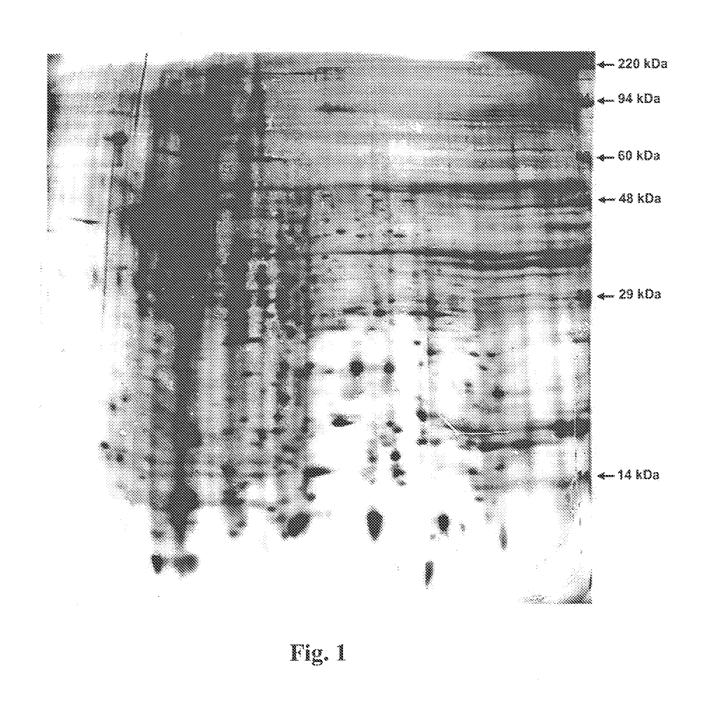

Secreted proteins and cell-supernatant fractions from CF-SC and exCF-SC (such as, ABM-SC and exABM-SC) conditioned media can be manufactured from serum-free conditions, concentrated and prepared in such manner as to make them suitable for in vivo use. When prepared this way, conditioned serum-free media from ABM-SC has been demonstrated to contain numerous pro-regenerative cytokines, growth factors, and matrix proteases in therapeutically effective concentrations (see, e.g., Table 1A, 1B and 1C). The complex mixture of hundreds of soluble factors produced by ABM-SC can be distinguished by 2D SDS PAGE (see, FIG. 1). Individual proteins and other macromolecules can be excised from these gels and identified using techniques routinely practiced in the art, such as, for example, MALDI-TOF mass spectrometry (Matrix Assisted Laser Desorption Ionisation-Time Of Flight spectrometry).

Utilizing the methods disclosed (as well as other separation techniques such as chromatography or hollow fiber cell culture systems), the desired proteins or cell supernatant fractions can be isolated, dialyzed, lyophilized and stored as a solid, or reconstituted in an appropriate vehicle for therapeutic administration. In one embodiment, the proteins or cell-supernatant fractions would be reconstituted in a semi-solid collagen or fibrin-based vehicle, and applied topically to the wound bed.

In addition to products generated by CF-SC and exCF-SC (such as, ABM-SC and exABM-SC), any number and type of pharmaceutically acceptable compound, such as small molecules to large macromolecular compounds (including biologics such as lipids, proteins, and nucleic acids) may be incorporated for administration with a pharmaceutically acceptable carrier such as biodegradable matrices in which CF-SC and exCF-SC (such as, ABM-SC and exABM-SC), or products generated by such cells, are contained. As a very small sampling, such additional molecules may include small molecule pharmaceuticals such as anti-inflammatories, antibiotics, vitamins, and minerals (such as calcium) to name but a few categories. Likewise, a very small sampling of biologics may include extracellular matrix proteins, blood plasma coagulation proteins, antibodies, growth factors, chemokines, cytokines, lipids (such as cardiolipin and sphingomyelin), and nucleic acids (such as ribozymes, anti-sense oligonucleotides, or cDNA expression constructs); including therapeutically beneficial variants and derivatives of such molecules such as various isoforms, fragments, and subunits, as well as substitution, insertion, and deletion variants. These are mentioned merely by way of example, as it can be appreciated by those skilled in the art that, in combination with the teachings provided herein, any number of additional structural or therapeutically beneficial compounds could be included for administration with a pharmaceutically acceptable carrier such as biodegradable matrices in which CF-SC and exCF-SC (such as, ABM-SC and exABM-SC), or products generated by such cells, are contained.

One embodiment of the invention encompasses a method of stimulating wound closure in a diabetic patient, such as a diabetic foot or venous leg ulcer, or a post-surgical wound. Stimulation of wound closure may be promoted by treatment with a pharmaceutical composition of CF-SC and exCF-SC (such as, ABM-SC and exABM-SC), or products generated by such cells, combined with naturally occurring extracellular matrix and/or blood plasma proteins components such as, for example, purified natural or recombinant human, bovine, porcine, or recombinant collagens, laminins, fibrinogen, and/or thrombin. The pharmaceutical composition may be administered to a mammal, including a human, at the site of tissue damage. In another embodiment, a topically administered biodegradable matrix is formed from a mixture of components such as purified natural or recombinant collagen, fibrinogen, and/or thrombin, combined with allogeneic CF-SC and exCF-SC (such as, ABM-SC and exABM-SC).

In another embodiment of the invention, a pharmaceutical composition of allogeneic cells and matrix are cultured in vitro for an extended period of time (such as, for example, but not limited to 1 day to one month or longer), producing the de novo formation of connective tissue. In another embodiment of the invention, the biodegradable matrix is bovine collagen or polyglycolic acid (PGA). In another embodiment, the pharmaceutical composition is cultured in serum-free cell media under conditions of reduced oxygen tension, for example but not limited to, oxygen tension equivalent to about 4-5% O₂, 5% CO₂, and balanced with nitrogen.

In one embodiment, the invention encompasses a method of preparing a pharmaceutical composition comprising the steps:
(a) preparing a solution comprising soluble collagen, serum-free cell culture media supplemented with glutamine, sodium biocarbonate, and HEPES (optionally including supplementation with insulin, transferrin, and/or selenium);
(b) re-suspending CF-SC or exCF-SC (for example, ABM-SC or exABM-SC) in the solution; and,
(c) transferring the cell suspension to a tissue mold, or equivalent thereof, to congeal at 37° C., for example, when placed in a cell culture incubator.

The above method of preparing a pharmaceutical composition may additionally comprise the step of incubating the culture for an extended period of time (such as, for example but not limited to, 1-3 days or longer) under low oxygen tension conditions equivalent to about 4-5% O₂, 5% CO₂, and balanced with nitrogen.

In another embodiment, the invention encompasses a method of preparing a pharmaceutical composition comprising the steps of:
a) preparing a solution comprising fibrinogen and thrombin;
b) re-suspending CF-SC or exCF-SC (for example, ABM-SC or exABM-SC) in the solution; and,
c) administering the re-suspended solution to an open wound.

In another embodiment, the invention encompasses a method of preparing a pharmaceutical composition comprising the steps of:
a) preparing a solution comprising soluble collagen, serum-free cell culture media supplemented with glutamine, sodium biocarbonate, and HEPES (optionally including supplementation with insulin, transferrin, and/or selenium); and
b) mixing a fraction or fractions of cell-supernatant derived from CF-SC or exCF-SC (for example, ABM-SC or exABM-SC) into the solution; and,
c) transferring the solution to a tissue mold, or equivalent thereof, to congeal at 37° C., for example, when placed in a cell culture incubator.

The above method of preparing a pharmaceutical composition may additionally comprise the step of incubating the tissue mold, or equivalent thereof, under atmospheric oxygen tension conditions equivalent to about 18-21% O₂ and 5% CO₂

In another embodiment, the invention encompasses a method of preparing a pharmaceutical composition comprising the steps of:
a) preparing a solution comprising fibrinogen and thrombin;
b) mixing a fraction or fractions of cell-supernatant derived from CF-SC or exCF-SC (for example, ABM-SC or exABM-SC) into the solution; and,
c) administering the solution to an open wound.

In another embodiment, the present invention encompasses tissue regeneration, particularly in the treatment of tissue damage caused by: immune related disorders (such as autoimmune disorders); inflammation (including both acute and chronic inflammatory disorders); ischemia (such as myocardial infarction); traumatic injury (such as burns, lacerations, and abrasions); infection (such as bacterial, viral, and fungal infections); and, chronic cutaneous wounds. The present invention encompasses treatment of a diversity of damage and disorders, for example, but not limited to, neurological damage and disorders of the central nervous system (brain) and peripheral nervous system (e.g., spinal cord) (for example, such as may be caused by neurotrauma and neurodegenerative diseases). Another embodiment of the invention encompasses treatment of diseases and disorders requiring bone, connective tissue, and cartilage regeneration, chronic and acute inflammatory liver diseases, vascular insufficiency, and corneal and macular degeneration. Another embodiment of the invention encompasses treating cardiovascular and pulmonary damage and disorders (for example, such as myocardial ischemia and repair and regeneration of blood vessels). Another embodiment of the invention encompasses treating damage and disorders of pancreatic and hepatic tissue as well as other endocrine and exocrine glands. Another embodiment of the invention encompasses treating damage and disorders of thymus as well as other immune cell producing and harboring organs. Another embodiment of the invention encompasses treating damage and disorders of the genitourinary system (for example, such as the ureter and bladder). Another embodiment of the invention encompasses treating hernias and herniated tissues. Another embodiment of the invention encompasses treatment, repair, regeneration, and reconstruction of heart valves.

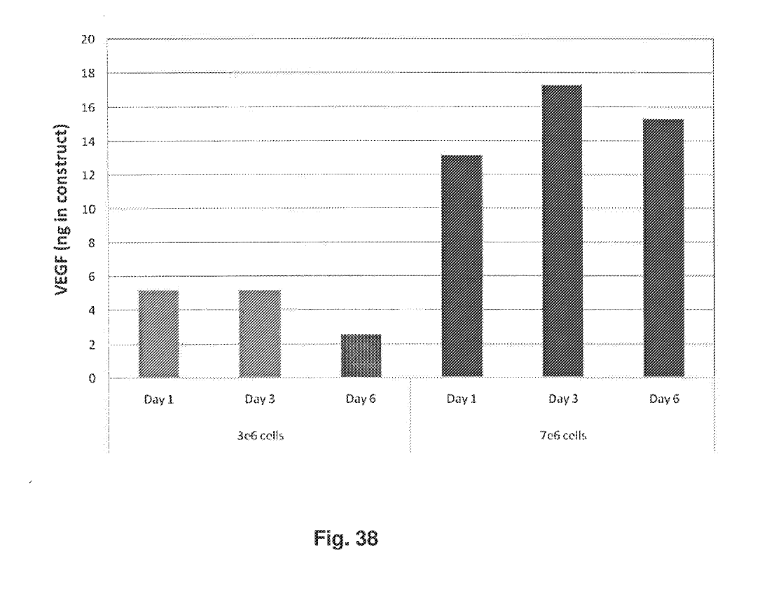

CF-SC and exCF-SC (such as, ABM-SC and exABM-SC) or protein and cell-supernatant fractions derived from CF-SC and exCF-SC (such as, ABM-SC and exABM-SC), can also be reconstituted in a solid-like collagen-base device. When the cells are reconstituted in such manner, the solid-like collagen matrix is remodeled over several days, giving rise to a neotissue that has fabricated its own unique matrix. Such CF-SC and exCF-SC (such as, ABM-SC and exABM-SC) derived neotissues are pliable, suturable, and bioactive (see e.g., FIG. 38). These structures could also be sterilized, chemically cross-linked, freeze-dried, or further processed, rendering the cells non-viable and incapable of further growth.

Such devices may be particularly beneficial in the treatment of burns, including full thickness burn wounds. To rebuild a vascularized wound bed, patients with severe burns are often treated with an artificial dermal replacement after surgical resection of the dead tissue. After the wound bed has healed, these patients are subsequently treated with artificial skin products or applications of epithelial cells in an attempt to re-grow host epidermis.

Compositions, such as described herein, when used in lieu of a conventional artificial dermal products (e.g., DERMAGRAFT™), may increase the longevity of subsequently grafted allogeneic skin, by inhibiting or reducing undesirable T-cell mediated immune reactions (see, e.g., Example 5). By modulating T-cell mediated immune responses, compositions of the present invention may permit subsequent reapplication of the artificial skin for a durations adequate to stimulate re-growth of the patients own skin.

The above-referenced ABM-SC have been shown to exhibit the following properties:

### In Vitro

Secretion of cytokines important in angiogenesis and tissue repair.

Release of factors for prevention and inhibition of scarring and matrix turnover.

Promotion of migration of endothelial cells indicative of pro-angiogenic activity.

### In Vivo

Significant improvement in outcomes in multiple animal models of acute myocardial infarction (AMI) and stroke.

Effective and well-tolerated intracardiac or intracerebral delivery of cells.

Cells not detectable in tissues eight weeks post-injection.

No measurable immune response against cells.

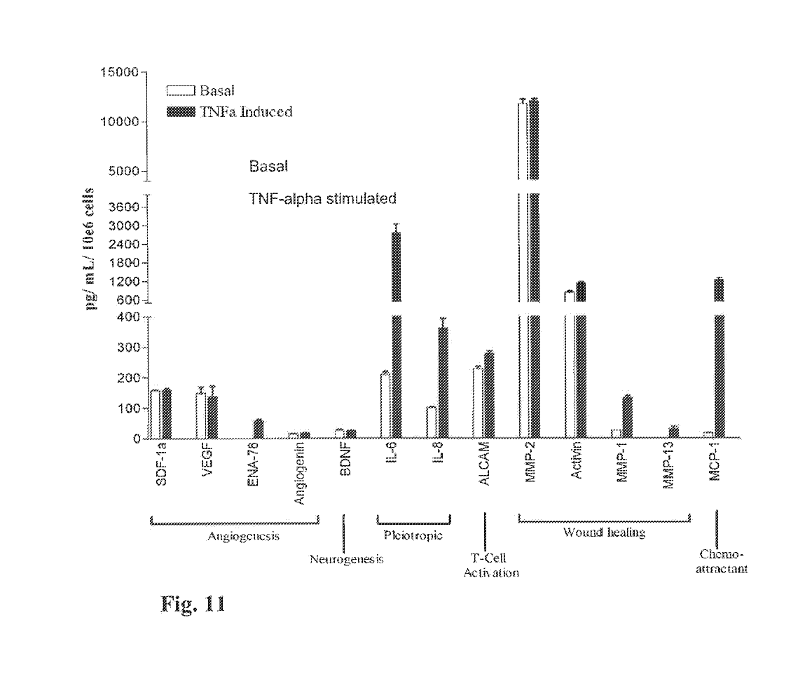

In one embodiment of the invention, a number of pro-regenerative cellular factors secreted by CF-SC and exCF-SC (such as, ABM-SC and exABM-SC) may be used in treatment, repair, regeneration, and/or rejuvenation of damaged tissues and organs (such as, for example, cardiac and neuronal organs and tissues damaged by, for example, heart failure due to acute myocardial infarction (AMI) or stroke). These include factors which can be secreted by CF-SC such as ABM-SC as shown in FIG. 11. For example, these factors include, but are not limited to, SDF-1alpha, VEGF, ENA-78, Angiogenin, BDNF, IL-6, IL-8, ALCAM, MMP-2, Activin, MMP-1, MMP-13, MCP-1. See, FIG. 11. Additional factors, such as those listed in Table 1A, 1B and 1C, may also be secreted by CF-SC and exCF-SC (such as, ABM-SC and exABM-SC).

Secretion of pro-regenerative factors by CF-SC and exCF-SC (such as, ABM-SC and exABM-SC) may be enhanced or induced by pre-treatment with stimulatory factors (such as, for example, tumor necrosis factor-alpha (TNF-alpha)) to induce the production of conditioned cell culture media or to prime the cells before administration of cells to a patient.

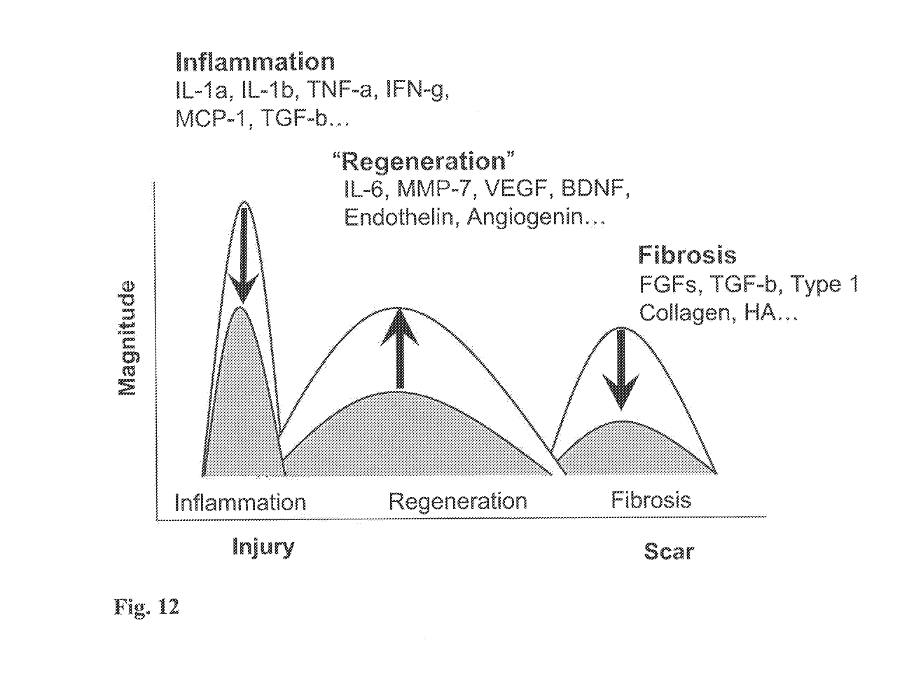

Acute ischemia, trauma or inflammation lead to a constellation of cellular and chemical events in the affected organs and tissues. See e.g., FIG. 12. In the inflammation phase there occurs a release of factors and an influx of cells to the injured site. In the regeneration phase there occurs a recruitment of circulating cells for the proper repair of functional tissue. And, in the fibrosis phase, there occurs a deposition of fibrotic scars which potentially compromise organ function. Moreover, a variety of cytokines and other biological molecules play a diversity of roles in each of these processes. See e.g., FIG. 12.

Use of CF-SC and exCF-SC (such as, ABM-SC and exABM-SC) in the present invention includes methods of treating and preventing inflammation, methods of stimulating organ and tissue regeneration while reducing fibrosis (i.e., tissue scarring), and methods of stimulating angiogenesis via compositions (e.g., cytokines, proteases, extracellular matrix proteins, etc) produced by stimulated or unstimulated CF-SC and exCF-SC (such as, ABM-SC and exABM-SC).

In another embodiment, CF-SC and exCF-SC may inhibit the biological process of fibrosis. Fibrosis is a natural byproduct of wound healing, scarring, and inflammation in many human tissues. Fibrosis, also known as fibrotic scarring, is a significant impediment to regenerating tissue with optimal function, especially in the heart and central nervous system (CNS), because scar tissue displaces cells needed for optimal organ function. Treatment with cells disclosed herein helps to prevent or reduce fibrosis and thereby facilitates the healing of damaged tissue. The fibrosis may be prevented by additive or synergistic effects of two or more secreted proteins or cell produced compositions, including membrane bound cell-surface molecules. Additionally matrix proteases induced or produced by the administered CF-SC and exCF-SC (such as, ABM-SC and exABM-SC) may play an important part in preventing fibrosis.

In another exemplary use of the present invention, angiogenesis, also known as neovascularization, is increased in a desired tissue. Angiogenesis, or the formation of new blood vessels, is a key component of regenerative medicine because newly formed tissue must have a blood supply, and angiogenesis is crucial if endothelial cells are lost during degenerative processes, disease progression, or acute injuries for which the present invention is a treatment. Hence, use of CF-SC and exCF-SC (for example, ABM-SC and exABM-SC) or compositions produced by such cells are useful in stimulating angiogenesis in target tissues and organs (especially, for example, in damaged cardiac tissue). Angiogenesis is an important component of tissue repair and can operate in conjunction with fibrosis inhibition to optimize healing of damaged tissues.

Another exemplary use of the present invention involves the stimulation of regeneration or rejuvenation processes without the engraftment of the administered cells. In vivo studies have shown that long term cell engraftment or tissue-specific differentiation of human ABM-SC or exABM-SC are generally not seen, suggesting that the mechanism by which these cells incite tissue regeneration is not through cell replacement, but instead through a host response to the cells themselves and/or factors they produce. This is not surprising, however, given that the role of ABM-SC in bone marrow is to provide structural and trophic support. Hence, the present invention includes treatment of damaged tissues and organs wherein the administered CF-SC and exCF-SC (for example, ABM-SC and exABM-SC) do not exhibit permanent or long-term tissue or organ engraftment. Instead, the therapeutic CF-SC and exCF-SC (for example, ABM-SC and exABM-SC) provide trophic support factors, suppress cell-death, inhibit fibrosis, inhibit inflammation (e.g., immune cell inflammatory responses), promote extra-cellular matrix remodeling, and/or stimulate angiogenesis without becoming part of the repaired tissue at a significant or currently detectable level.

A further example of the present invention teaches that after a period of time, the administered cells are not detected anywhere in the experimental animal, suggesting the administered cells are completely cleared from the body. This suggests that secreted factors play an essential role in the repair of damaged tissue.

In yet another example of the present invention illustrating its utility, the hABM-SCs disclosed herein come from one donor source. As such, these cells will be allogeneic cell transplants in patients which might suggest that these transplanted cells could potentially stimulate an adverse immune response. However, surprisingly, transplanted allogeneic cells disclosed herein actually can suppress mitogen induced T-cell proliferation in vitro and avoid induction of a T-cell-dependent immune response in vivo. A T-cell mediated immune response is a key factor in immune processes that are detrimental to healing, regenerative, and rejuvenation processes.

As used herein “an effective amount” is an amount sufficient to produce detectable improvement in tissue, organ, or biological system (e.g., immune system) performance, function, integrity, structure, or composition wherein said improvement is indicative of complete or partial amelioration, restoration, repair, regeneration, or healing of the damaged tissue, organ or biological system.

Table 1A, 1B and 1C shows an extensive list of cytokines, growth factors, soluble receptors, and matrix proteases secreted by human ABM-SC when sub-cultured in serum-free cell culture media. Media Supernatant Concentrate #1=Advanced DMEM (Gibco™) supplemented with 4 mM L-glutamine. Media Supernatant Concentrate #2=RPMI-1640 containing 4 mM L-glutamine and HEPES (HyClone) supplemented with Insulin-Transferrin-Selenium-A (Gibco™).

The results demonstrate that numerous trophic factors and soluble receptors important for tissue regeneration and modulation of the immune system are produced by ABM-SC at therapeutically relevant levels when cultured under these conditions. Notably, earlier experiments demonstrated that supplementation of the base culture medium with insulin, transferrin, and selenium was required to achieve secreted protein levels such as those indicated in Table 1A, 1B and 1C.

### Living and Non-Living Bioactive Devices

Embodiments of the invention include generation of CF-SC and/or exCF-SC seeded scaffolds that create tissue-like constructs able to produce soluble factors and matrix deposition within constructs for enhancing wound healing. Scaffold properties, cell seeding, and culture conditions will be evaluated and optimized to produce tissue engineered constructs useful in aiding repair and regeneration of tissues such as skin, bone, nerve, and muscle. These tissue engineered constructs can be used as products for delivering therapeutically relevant factors to the injured/damaged tissues in vivo.

Specifically, human or non-human CF-SC and/or exCF-SC can be embedded within collagen hydrogel scaffolds for creation of tissue engineered constructs. Cell seeded collagen gel constructs can be maintained in culture in vitro to modulate or stimulate cells to secrete and produce relevant factors into the constructs. Culture conditions/parameters can possibly be varied with chemical, mechanical, or electrical stimulation (e.g., low oxygen tension, growth factor addition, or culture vessel agitation). Human, porcine, or bovine derived collagen, for example but without limitation, can be useful in generating these products. These constructs can further be developed with combination or replacement of collagen with other naturally derived matrices including fibrin, hyaluronic acid, heparin, alginate, gelatin, chitosan, laminin, or fibronectin.

Tissue engineered constructs can also be generated from human or non-human CF-SC and/or exCF-SC seeded synthetic polymer scaffolds. Specifically, FDA approved materials such as poly lactic-co-glycolic acid co-polymers will be used due to their good biocompatibility and biodegradation. These co-polymers can be produced into multiple scaffold formations with specific properties. Degradation rates can be tailored by varying co-polymer ratios of lactic to glycolic acid. Specific arrangements of these polymers useful as a tissue engineered construct include, but are not limited to, porous non-woven meshes. CF-SC and/or exCF-SC seeded polymer scaffolds can be maintained in culture similarly to the collagen constructs and be optimized using the same manipulations as described above. Also, these constructs can be generated with the addition or incorporation of naturally derived matrices into the polymer scaffold. Other synthetic polymers useful as scaffolds for tissue engineered constructs with human or non-human CF-SC and/or exCF-SC include non-degradable silicone, poly-tetrafluoroethylene, poly-dimethylsiloxane, polysulfones and degradable polyethylene glycol, polycaprolactone, and other polyesters or polyurethanes.

The human or non-human CF-SC and/or exCF-SC seeded scaffolds can be cultured to form neotissues in vitro. These constructs can be used as living constructs for direct application or cryopreservation and later delivery to a human or non-human subject or can further be manipulated and transformed into non-living constructs for storage and later application to the patient. Living constructs can include cell suspensions in liquid or semi-solid matrices for injection, cell seeded matrix particulates for injection, and cell seeded solid constructs for implantation. These constructs can be cryopreserved from the time of production until application to the subject in order to maintain constructs with viable cells and intact proteins. Also, these same formulations can be further processed to produce constructs rendered non-living leaving constructs with non-viable cells, but still preserving the therapeutically relevant factors and matrix produced by the cells within the construct. Methods to render constructs non-living include chemical modifications such as irradiation, protein cross-linking, additives for protein stabilization, decellularization or temperature manipulations such as freezing, dehydrothermal drying, and lyophilization. Specific chemical cross-linking treatments include glutaraldehyde, carbodiimides (EDC), polyepoxide compounds, diisocyanates, divinyl sulfone, and naturally-derived genipin or ribose. Sterilization methods might also be further manipulations used on the tissue engineered constructs to render non-living or for terminal sterilization; methods include irradiation, electron beam, or gas plasma treatments. Non-living constructs may be preserved and stored at room temperature.

Embodiments of the invention include bioactive devices (i.e., compositions, articles, objects, manufactures, ensembles, collections, products, etc.) wherein the devices are “living” (“live”), “non-living,” or a combination of both living and non-living manufactures comprised of live CF-SC and exCF-SC, non-living CF-SC and exCF-SC, or a mixture of both live and non-living CF-SC and exCF-SC in any combination. Embodiments of the invention further include such living and non-living devices wherein the devices are comprised either partially or entirely of components derived (with or without additional purification, isolation, and/or separation steps) from living and/or non-living CF-SC and exCF-SC. As defined herein, “non-living” devices are: (a) devices that contain no living CF-SC and/or exCF-SC; (b) devices that contain CF-SC and/or exCF-SC which have been subjected to a treatment condition intended to kill them (e.g., irradiation, freezing, freeze-thaw, air-dry/dessication, chemical cross-linking, heating, freeze-drying) wherein said treatment may or may not have been 100% effective (i.e., some fraction of living CF-SC and/or exCF-SC remain); and, (c) devices that contain one or more components derived from (e.g., separated, isolated, or purified) from living or non-living CF-SC and/or exCF-SC. As defined herein, a “living” device is a device that contains live CF-SC and/or exCF-SC wherein said device has been subjected to treatment conditions intended to maintain the viability of CF-SC and/or exCF-SC therein. A “living” device as defined herein may comprise, in part, some portion of non-living CF-SC and/or exCF-SC (i.e., CF-SC and/or exCF-SC that are dead).

In one embodiment of the invention a living device comprises nearly 100% living CF-SC and/or exCF-SC. In other embodiments of the invention a living device comprises about: 25% or more, 50% or more, 60% or more, 70% or more, 75% or more, 80% or more, 85% or more, 90% or more, 95% or more, 98% or more, and 99% or more living CF-SC and/or exCF-SC.

In one embodiment of the invention a non-living device may comprise 100% or nearly 100% non-living (i.e., dead) CF-SC and/or exCF-SC. In other embodiments of the invention a non-living device may comprise about: 25% or less, 20% or less, 15% or less, 10% or less, 5% or less, 2% or less, and 1% or less non-living CF-SC and/or exCF-SC.

Embodiments of the invention further include bioactive devices which are cost-effective and easy to assemble (by those skilled in the art) upon acquisition of the necessary component parts.

Embodiments of the invention include production and use of bioactive devices of dermal-like tissue constructs comprised of: (1) a biodegradable scaffold (for example, a scaffold comprised of polyglycolic acid (PGA)); and, (2) living, non-living, or a mixture of living and non-living CF-SC and/or exCF-SC, or comprised of one or more components derived from CF-SC and/or exCF-SC (living or non-living).

Embodiments of the invention include bioactive devices which have been selected, engineered, or modified to achieve a desired rate of biodegradation. Embodiments of the invention include biodegradation of the scaffolds or bio-engineered constructs wherein approximately 100%, 98%, 95%, 90%, 85%, 80% or 50% of the original volume or mass of the scaffold or bio-engineered construct has been eliminated, absorbed, deteriorated, or otherwise dismantled in 6 months or less, 3 months or less, 1 month or less, 3 weeks or less, 2 weeks or less, 1 week or less, 5 days or less, 3 days or less, 48 hours or less, 24 hours or less, 12 hours or less.

Additional embodiments of the invention include methods for testing and assessing different materials for biocompatibility and bioactivity when used in conjunction with CF-SC and/or exCF-SC and components derived from CF-SC and/or exCF-SC. Methods for testing biocompatibility include, but are not limited to, for example, testing cell viability, cell growth and/or proliferation, metabolism, survival, and apoptotic activity. Examples of methods and techniques that may be used for such assessments include, but are not limited to, Calcein/EthD-1 assays, CELL TITER GLO™ assays, glucose/lactate assays, histology assessments. Methods for testing bioactivity include, but are not limited to, for example, testing for secretion of trophic factors and matrix turnover. Examples of methods and techniques that may be used for such assessments include, but are not limited to, ELISAs, measurement of matrix content, immunostaining, and histology assessments.

Embodiments of the invention encompass alteration of cell seeding density and cell culture conditions to generate devices containing desired therapeutic levels of secreted and endogenous factors. Assays used to assess such devices include, but are not limited to, for example, visual inspection/handling, cell counts and viability, histology assessments, trophic factor and matrix production measurements (e.g., using ELISAs or matrix kits), and functional behavior (e.g., gel contraction, cell co-culture assays).

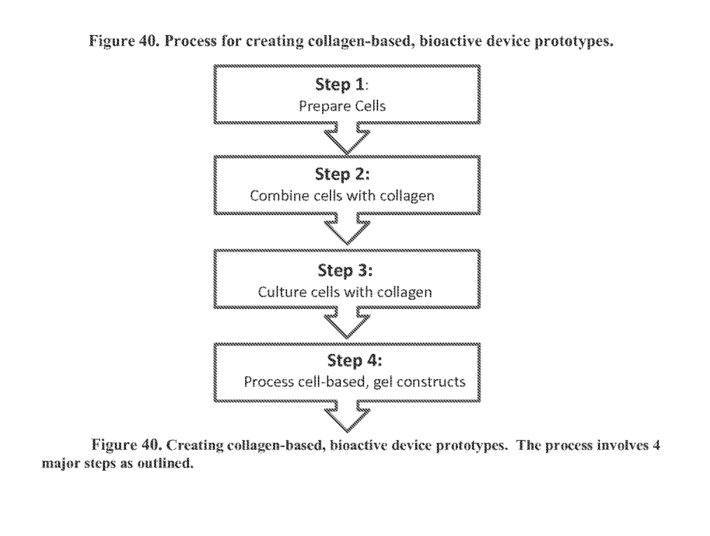

The invention further provides methods of making collagen-based bioactive devices. FIG. 40 is a flowchart illustrating an embodiment of a process for making collagen-based bioactive devices. In step 1, cells are prepared by methods of the invention; in step 2, the cells are combined with collagen as disclosed herein; in step 3, the cells are cultured with collagen as described herein and in step 4 the constructs are processed. For example, in embodiments, cells of the invention are encapsulated in a biomatrix, e.g., collagen, gel solution. Once the gel solution is solidified, in embodiments the construct is cultured under low oxygen conditions. At the end of the culture period, in embodiments, the constructs are processed by crosslinking, with, for example, glutaraldyde, followed by washing with, for example, glycine. In embodiments, the constructs are dehydrated, rendering the cells inactive while preserving the bioactive factors secreted by the cells. The constructs can be used as neotissue and/or a surgical implant either in the dehydrated state, or after rehydration. Dehydration encompasses full dehydration, i.e., all liquid evaporated from the constructs under ambient conditions, but does not necessarily encompass dehydration to a specific humidity level below ambient humidity.

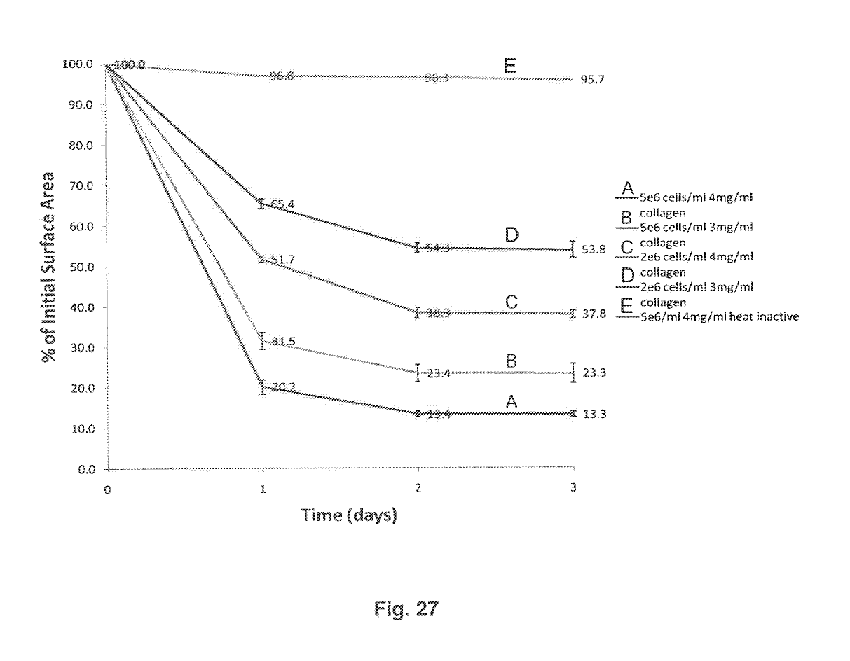

An example of embodiments of the invention include, but are not limited to, combining CF-SC and/or exCF-SC with collagen (e.g., rat or porcine collagen) at final concentrations of about 2×10⁶ cells/mL, about 5×10⁶ cells/mL or about 6×10⁶ cells/mL with about 3 mg/mL, about 4 mg/mL or about collagen. See, FIG. 27. FIG. 27 demonstrates results with human exCF-SC seeded at varying densities within medical-grade porcine collagen gels (e.g., THERACOL™; SEWONCELLONTECH, Seoul, Korea) at either 3 mg/ml or 4 mg/ml concentrations and cultured suspended in media (n=3 for each condition at each time point). Diameters of the gel constructs were measured at 24, 48, and 72 hrs. Percent surface area contraction was calculated by comparing x and y dimension initial diameters to contracted diameters of each time point. A control gel containing heat inactivated cells showed little contraction. In contrast, there was a dose response of collagen gel contraction with increasing cell density and also with increasing collagen gel concentration.

Embodiments of the invention further comprise combining CF-SC and/or exCF-SC with collagen (or another biocompatible matrix) at final concentrations ranging from about 1×10³ cells/mL to about 1×10⁷ cells/mL. For example, embodiments of the invention may comprise collagen (or another biocompatible matrix) with cells at a final concentration of about: 1×10³ cells/mL or greater, 1×10⁴ cells/mL or greater, 1×10⁵ cells/mL or greater, 1×10⁶ cells/mL or greater, 1×10⁷ cells/mL or greater, 2×10³ cells/mL or greater, 2×10⁴ cells/mL or greater, 2×10⁵ cells/mL or greater, 2×10⁶ cells/mL or greater, 3×10³ cells/mL or greater, 3×10⁴ cells/mL or greater, 3×10⁵ cells/mL or greater, 3×10⁶ cells/mL or greater, 4×10³ cells/mL or greater, 4×10⁴ cells/mL or greater, 4×10⁵ cells/mL or greater, 4×10⁶ cells/mL or greater, 5×10³ cells/mL or greater, 5×10⁴ cells/mL or greater, 5×10⁵ cells/mL or greater, 5×10⁶ cells/mL or greater, 6×10³ cells/mL or greater, 6×10⁴ cells/mL or greater, 6×10⁵ cells/mL or greater, 6×10⁶ cells/mL or greater, 7×10³ cells/mL or greater, 7×10⁴ cells/mL or greater, 7×10⁵ cells/mL or greater, 7×10⁶ cells/mL or greater, 8×10³ cells/mL or greater, 8×10⁴ cells/mL or greater, 8×10⁵ cells/mL or greater, 8×10⁶ cells/mL or greater, 9×10³ cells/mL or greater, 9×10⁴ cells/mL or greater, 9×10⁵ cells/mL or greater, 9×10⁶ cells/mL or greater.

Embodiments of the invention may also comprise CF-SC and/or exCF-SC (or components derived therefrom) at any final concentration combined with collagen (or another biocompatible matrix) at concentrations ranging from about 0.1 mg/mL to about 50 mg/mL. For example, embodiments of the invention may comprise CF-SC and/or exCF-SC (or components derived therefrom) with collagen (or another biocompatible matrix) at a concentration of about: 0.1 mg/mL or greater, 0.5 mg/mL or greater, 1 mg/mL or greater, 2 mg/mL or greater, 3 mg/mL or greater, 4 mg/mL or greater, 5 mg/mL or greater, 6 mg/mL or greater, 7 mg/mL or greater, 8 mg/mL or greater, 9 mg/mL or greater, 10 mg/mL or greater, 12 mg/mL or greater, 15 mg/mL or greater, 20 mg/mL or greater, 25 mg/mL or greater, 30 mg/mL or greater, 40 mg/mL or greater, 50 mg/mL or greater.

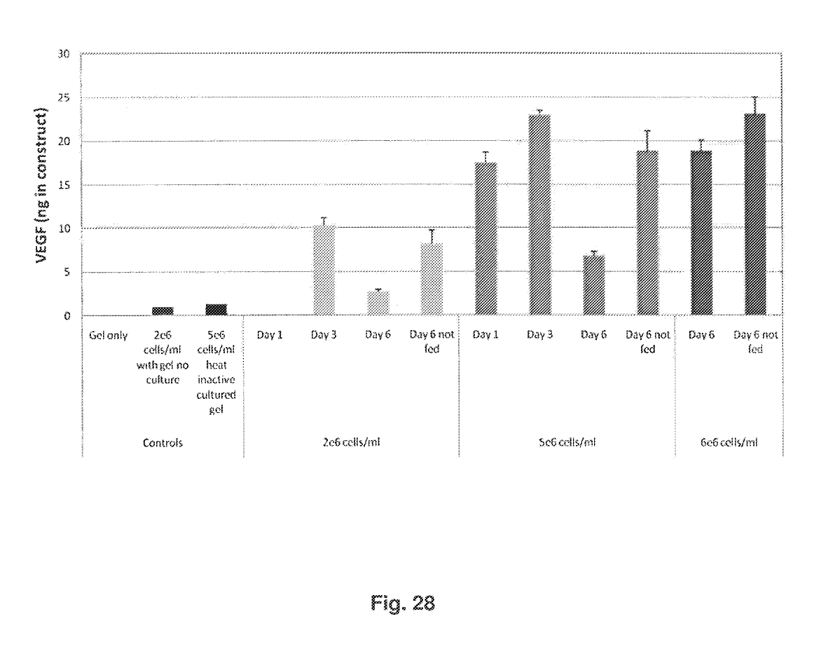

Embodiments of the invention include combining CF-SC and/or exCF-SC with a biocompatible matrix (for example, but not limited to collagen) at a combined final collagen concentration, cell concentration, and duration, optimized to provide a desired level of trophic factor production/concentration (for example, but not limited to VEGF). See e.g., FIG. 28.

FIG. 28 depicts results obtained with human exCF-SC seeded at varying densities of 2e6, 5e6, or 6e6 cells/ml within 4 mg/ml medical-grade porcine-collagen gel neotissue and cultured suspended in media for either 1, 3, or 6 days (n=3 for each condition at each timepoint). Constructs were replenished with fresh media every other day or where indicated were not replenished for 6 day cultures. At each timepoint, gels were washed 3× in balanced salt solution and snap frozen. Lysates were made of each gel by mechanical dissociation in protein extraction buffer. ELISA was used on gel lysates to quantify amount (in ng) of VEGF contained within hABM-SC collagen gel constructs (error bars represent standard deviation of 3 separate gels). Controls include gel only with no culture, 2e6 cells/ml seeded gel with no culture, and 5e6 heat inactivated cells/ml seeded gel 6 day culture. Results indicate an increase in VEGF contained within the gels with increasing cell densities. A culture time of 3 days indicates maximal VEGF within the hABM-SC seeded collagen gels.

As exemplary embodiments of the invention, but without limitation, CF-SC and/or exCF-SC may be combined at any cell concentration described herein, with collagen at any concentration described herein, for a duration in a range of about 1 to about 30 days. For example, the above-reference duration may be, without limitation, about: 1 day, 2 days, 3 days, 4 days, 5 days, 6 days, 7 days, 8 days, 9 days, 10 days, 11 days, 12 days, 13 days, 14 days, 15 days, 20 days, 25 days, and 30 days.

In embodiments of the invention, the gel neotissue, or gel construct comprises a total gel volume of from 1 ml to 20 ml, 2 ml to 10 ml, 3 ml to 8 ml or 5 ml to 7 ml. In embodiments, the total gel volume is at least 1 ml, at least 2 ml, at least 3 ml, at least 4 ml, at least 5 ml, at least 6 ml, at least 7 ml, at least 8 ml, at least 9 ml, at least 10 ml, at least 11 ml, at least 12 ml, at least 13 ml, at least 14 ml, at least 15 ml, at least 16 ml, at least 17 ml, at least 18 ml, at least 19 ml, and at least 20 ml.

Embodiments of the invention include, but are not limited to biocompatible matrices and scaffolds such as a SCAFTEX™ PLGA scaffold (BMS, BioMedical Structures, LLC, RI, USA).

Embodiments of the invention include, but are not limited to, CF-SC and/or exCF-SC cultivated in biocompatible matrices and scaffolds with media which contain serum, with media which contain supplemental growth and/or survival factors, with media which is protein-free (i.e., chemically defined media), or with media which is serum-free (e.g., media containing protein supplements but not serum supplements).

Some examples, without limitation, of commercially available scaffold products include:

BIOBRANE™ (Bertek Pharmaceuticals Inc.),

PERMACOL™ (Tissue Science Laboratories, Inc.),

STRATTICE™ (LifeCell Corp.),

E-Z-DERM™ (Brennen Medical, Inc.),

MATRISTEM™ (Medline Industries, Inc.),

INTEGRA™ and INTEGRA™ Flowable Wound Matrix (Integra LifeSciences Corp.),

PRIMATRIXT™ (TEI Biosciences),

TISSUEMEND™ (TEI Biosciences),

ALLODERM™ (LifeCell Corp.),

CYMETRA™ (LifeCell Corp.),

NEOFORM™ (Mentor Corp.),

DermaMatrix (Musculoskeletal Transplant Foundation™ (MTF™)),

GRAFTJACKET™ and GRAFTJACKETXPRESS™ (LifeCell Corporation and Wright Medical Group),

GAMMAGRAFT™ (Promethean LifeSciences, Inc.),

ORTHADAPT™ Bioimplant (Pegasus Biologics, Inc.),

Some examples, without limitation, of commercially available scaffold products containing either living or non-living cells include:

CELADERM™ (Advanced BioHealing, Inc.),

LASERSKIN™ (Fidia Advanced Biopolymers S.R.L., Italy),

PERMADERM™ (Cambrex Corp.),

APLIGRAF™ (Organogenesis, Inc.),

ORCEL™ (Ortec International Inc., Israel),

DERMAGRAFT™ (Advanced Biohealing Inc.), and

TRANSCYTE™ (Advanced Biohealing Inc.).

Some examples, without limitation, of methods which may be used to generate non-living bioactive devices include subjecting cells or devices to treatments such as: cross-linking treatments (for example, using agents such as glutaraldehyde, carbodiimides, polyepoxide compounds, and divinylsulfone); lyophilization (which would also allow storage of devices at room temperature and function to preserves protein content); subjecting cells and or devices to one or more freeze/thaw cycles (e.g., such as is currently used for DERMAGRAFT™); and, decellularization (which can also aid in decreasing potential immunogenicity which may be caused by immunogenic peptides generated when cells and/or devices are subjected to freezing). In embodiments of the invention, at least one crosslinking agent, such as glutaraldehyde, is present in the cross linking reaction at concentration of 0.0009% to 0.09%, 0.001% to 0.08%, 0.005% to 0.05% and 0.008% to 0.08%. In embodiments, the crosslinking agent is present in the cross linking reaction at 0.01%, 0.05% or 0.005%.

The invention provides a device for implantation in a animal comprising CF-SC and/or exCF-SC and biocompatible and/or biodegradable matrix (for example, but not limited to collagen) at a combined final collagen concentration, cell concentration, and duration, optimized to provide a desired level of trophic factor production/concentration (for example, but not limited to VEGF). Such a device can be implanted during surgery, for example. In embodiments, the device has suitable physical properties, such as being flexible and durable in both the dehydrated state and rehydrated state. In exemplary embodiments, the devices are circular or approximately circular and have a diameter of at least 23 mm, at least 24 mm, at least 26 mm, at least 27 mm, at least 28 mm, at least 29 mm, at least 30 mm and at least 35 mm. In embodiments, the devices weigh at least 60 mg, at least 65 mg, at least 70 mg, at least 80 mg, at least 90 mg, at least 100 mg, at least 110 mg, at least 115 mg, at least 120 mg and at least 130 mg.

In embodiments, the devices of the invention are used in preventing or repairing orthopedic injuries in animals, including humans. In embodiments, orthopedic injuries include, but are not limited to, injuries are to the neck, arm, back, elbow, hand, foot, knee, wrist, hip, and ankle. For example, tendon injuries in animals, including humans, may be aided by attachment of the device to the area in need of repair. In embodiments, the devices of the invention are approximately rectangular. For example, circular pieces can be cut into strips for attachment to a part of the body. In embodiments, the tendon targeted for repair is in the hand of a human. In embodiments, the device is surgically implanted into the body of the injured animal, e.g., human. For example, the device of the invention can be used as an alternative to an epi-tendinous repair, in that it appears to effectively wrap around the tendon and provide a smooth gliding surface. Epi-tendinous repairs historically add 20% strength to the repair, such that the bioactive devices of the invention may preclude the use of this stitch. If additional factors are embedded within the device, both improved flexor tendon strength and diminished adhesion formation may be realized. In embodiments, the devices of the invention embedded with, for example, chondrocytes, are used as a spacer in thumb arthritis surgery (CMC Arthroplasty). There are currently two major grafts used for CMC joint surgery that are FDA approved and neither contain a bioactive component consisting of cartilage forming cells.

### Examples of Bioassays

A variety of bioassays are available which may be used to further optimize bioactivity and to study and further identify the mode of action by which cells, cell components, and bioactive devices of the invention perform. Some examples, without limitation, of such assays may include scratch assays, assessment of bioactivity using 3-dimensional skin constructs as in vitro model systems (such as those described in Am. J. Pathol., 156(1):193-200 (2000) and references cited therein), assessments of macrophage activation, and assessments of the effect of supplemental factors on the qualitative and quantitative secretion profiles of factors produced by cells and bioactive devices of the invention.

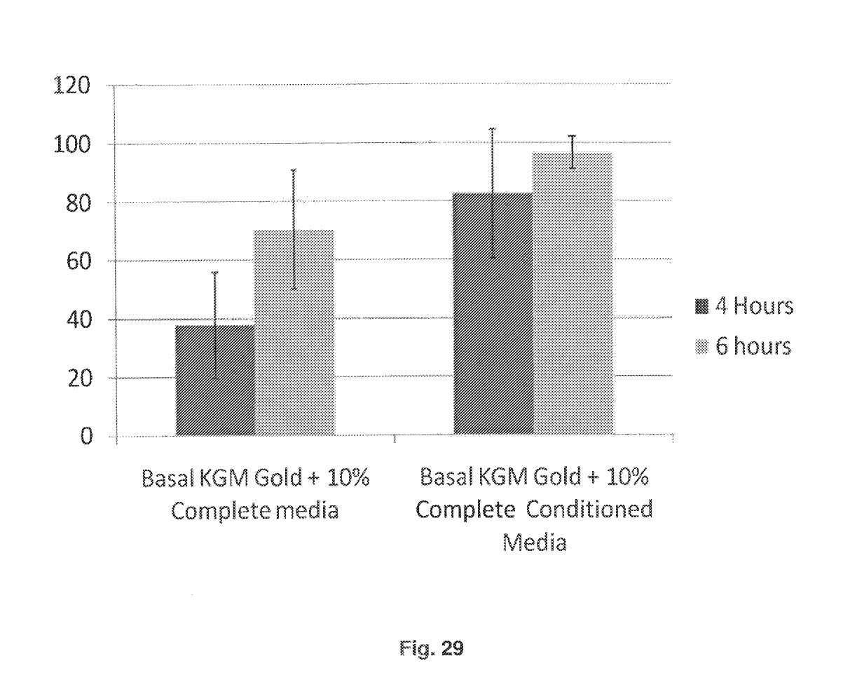

The scratch assay is an easy, low-cost and well-known method for measuring cell migration in vitro. The assay is performed by scratching a cell culture monolayer to create a void lacking adherent cells. Images of the void may be captured at the beginning and at regular intervals during cell migration as the void (i.e., the scratch) is closed by cells migrating and/or growing across the void. A comparison of images is then performed to quantify the migration rate of the cells using at least one experimental treatment method compared to a control treatment. See, FIG. 29.

FIG. 29 depicts results demonstrating that hABM-SC produce factors which enhance the rate and magnitude of closure in an in vitro wound closure assays. In particular, normal human keratinocytes (NHEKs) were grown to a confluent monolayer before being scratched with a pipet tip to create a scratch wound across the monolayer. Photographs were taken immediately after the scratch was made and at 4 and 6 hour intervals and incubated with control or conditioned media. The extent of the wound closure was determined by comparing the 4 and 6 hour photographs to the initial pictures using image analysis software (CMA, Muscale LLC) to calculate the scratch area. The extent of closure is depicted as the percentage of the initial scratch in this figure. Conditioned media with factors secreted by human exCF-SC cells (Complete Conditioned Media) increased the percentage of closure compared to control media (Complete Media) not exposed to hABM-SC cells. At both 4- and 6-hour time points, the scratch area remaining in wells treated with media conditioned by hABM-SC cells human exCF-SC was significantly reduced compared to those treated with control media (p<0.001 and p<0.01 respectively), demonstrating both an increased rate of closure and magnitude of closure.

Three-dimensional skin constructs (such as those described in Am. J. Pathol., 156(1):193-200 (2000) and references cited therein) can be used, for example, to analyze and optimize the effect of bioactive devices of the invention on skin growth, development, modeling, re-modeling, and wound repair.

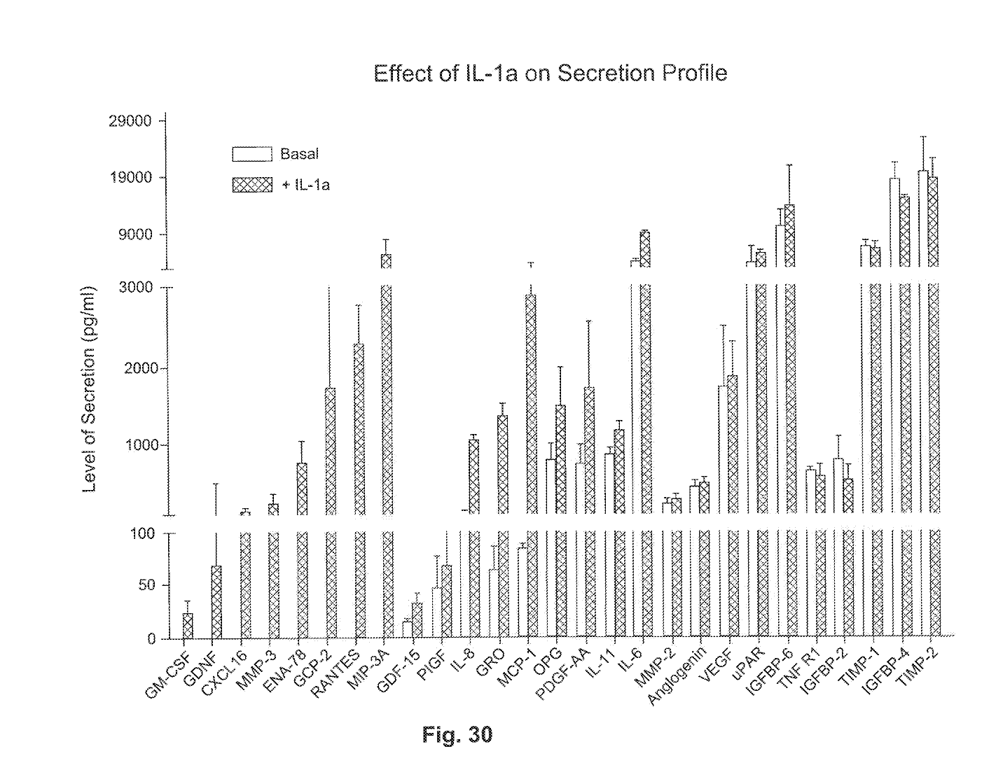

Assessment of the effect of supplemental factors on the qualitative and quantitative secretion profiles of proteins and other compounds produced by cells and bioactive devices of the invention may be performed using a variety of supplemental factors. As three examples, but without limitation, of such assessments, FIGS. 30, 31, and 32 shows the effect of IL-1alpha (IL-1a), TNF-alpha (TNFa), and Interferon-gamma (IFNg) on the secretion profile of factors produced by bone marrow-derived cells exposed or not exposed to exogenous treatments with these molecules.

FIG. 30 depicts results from a quantitative determination of secreted factors in conditioned media using QUANTIBODY™ glass antibody arrays from Ray Biotech Inc. (Norcross, Ga., USA) after human exCF-SC were exposed to IL-1 alpha (IL-1a) (10 ng/mL) for 24 hours. Calculations of the quantity of protein detected by each antibody were determined using a five point standard curve using Ray Biotech Inc.'s Q Analyzer software. Each antibody, together with a positive and negative control, was arrayed in quadruplicate. Outliers were removed automatically from the raw data via the Q Analyzer software and the mean values were determined to calculate the quantity of protein. Each bar represents the mean of three biological replicates±the standard deviation. Eight factors were detected only upon IL-1a stimulation (i.e., GM-CSF, GDNF, CXCL-16, MMP-3, ENA-78, GCP-2, RANTES, MIP-3a) while additional factors were induced by IL-1a treatment at least two fold above basal levels (e.g., GDF-15, IL-8, GRO, MCP-1). Because IL-1 alpha is present in inflammatory conditions, up-regulation of these factors by human exCF-SC is important for suppression of inflammation, angiogenesis, tissue regeneration and recruitment of immune effectors.

FIG. 31 depicts results from a quantitative determination of secreted factors in conditioned media using QUANTIBODY™ glass antibody arrays from Ray Biotech Inc. (Norcross, Ga., USA) after human exCF-SC were exposed to tumor necrosis factor alpha (TNFa) (10 ng/mL) for 24 hours. Calculations of the quantity of protein detected by each antibody were determined using a five point standard curve using Ray Biotech Inc.'s Q Analyzer software. Each antibody, together with a positive and negative control, was arrayed in quadruplicate. Outliers were removed automatically from the raw data via the Q Analyzer software and the mean values were determined to calculate the quantity of protein. Each bar represents the mean of three biological replicates±the standard deviation. Five factors were detected only upon TNFa stimulation (i.e., CXCL-16, ENA-78, ICAM-1, MIP-3a, RANTES) while an additional five factors were induced by TNFa treatment at least two fold above basal levels (i.e., GDF-15, PIGF, IL-8, GRO, MCP-1). Because TNFa is present in inflammatory conditions, up-regulation of these factors by hABMSCs is important for suppression of inflammation, angiogenesis, tissue regeneration and recruitment of immune effectors.

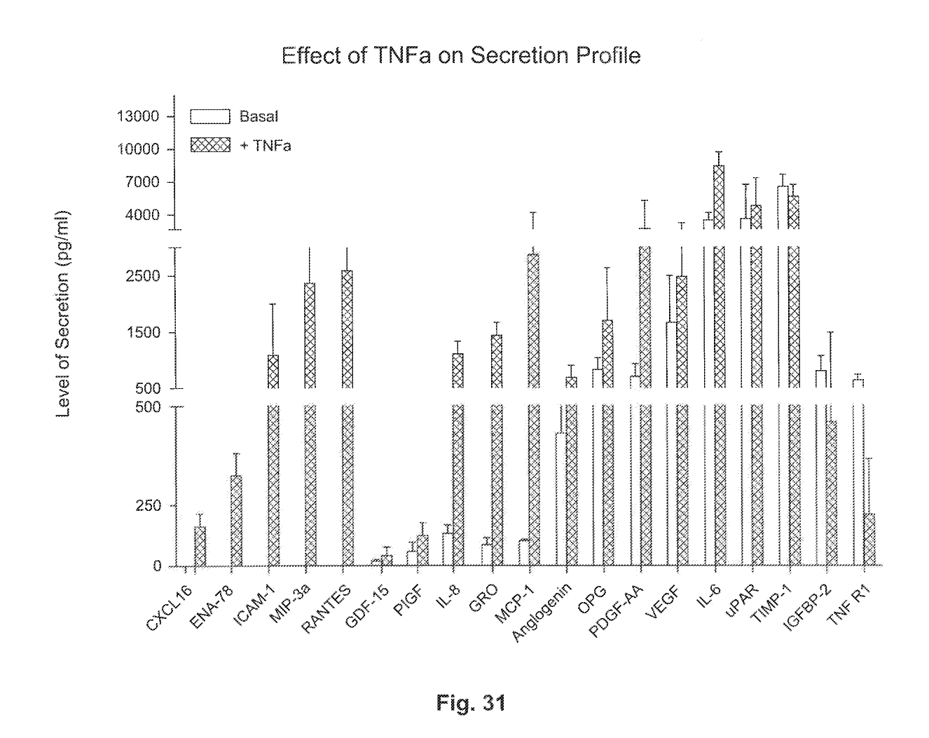

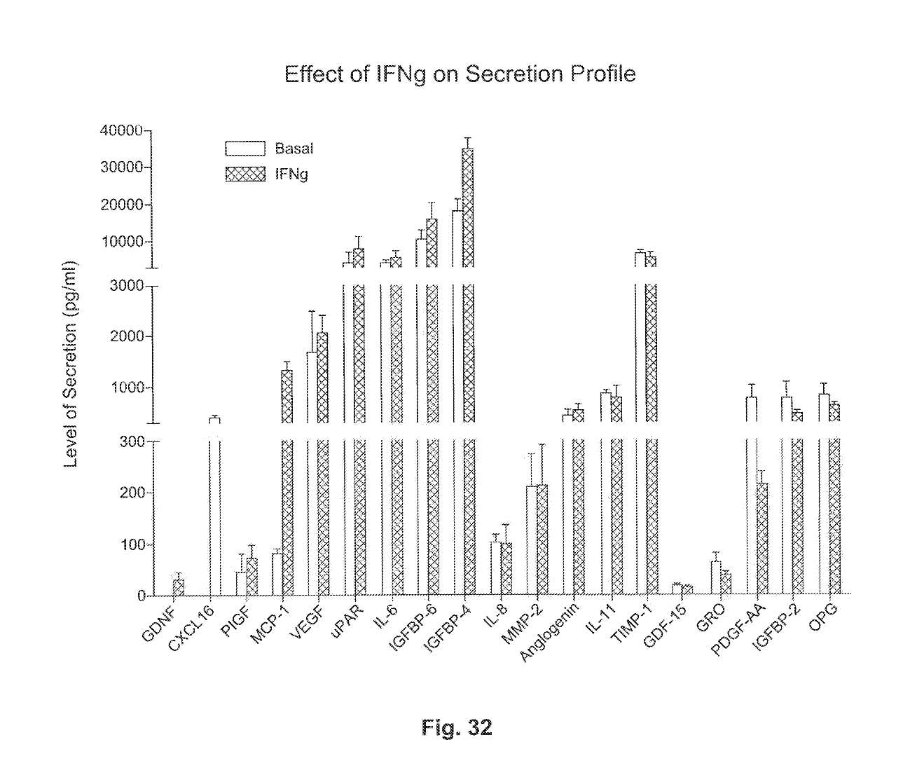

FIG. 32 depicts results from a quantitative determination of secreted factors in conditioned media using QUANTIBODY™ glass antibody arrays from Ray Biotech Inc. (Norcross, Ga., USA) after human exCF-SC were exposed to interferon gamma (IFNg) (10 ng/mL) for 24 hours. Calculations of the quantity of protein detected by each antibody were determined using a five point standard curve using Ray Biotech Inc.'s Q Analyzer software. Each antibody, together with a positive and negative control, was arrayed in quadruplicate. Outliers were removed automatically from the raw data via the Q Analyzer software and the mean values were determined to calculate the quantity of protein. Each bar represents the mean of three biological replicates±the standard deviation. Two factors are detected only upon IFNg stimulation (i.e., GDNF and CXCL16) while an additional two factors are induced by IFNg treatment at least two fold above basal levels (i.e., PIGF AND MCP-1). Because IFNg is present in inflammatory conditions, up-regulation of these factors by hABMSCs is important for suppression of inflammation, angiogenesis, tissue regeneration and recruitment of immune effectors.

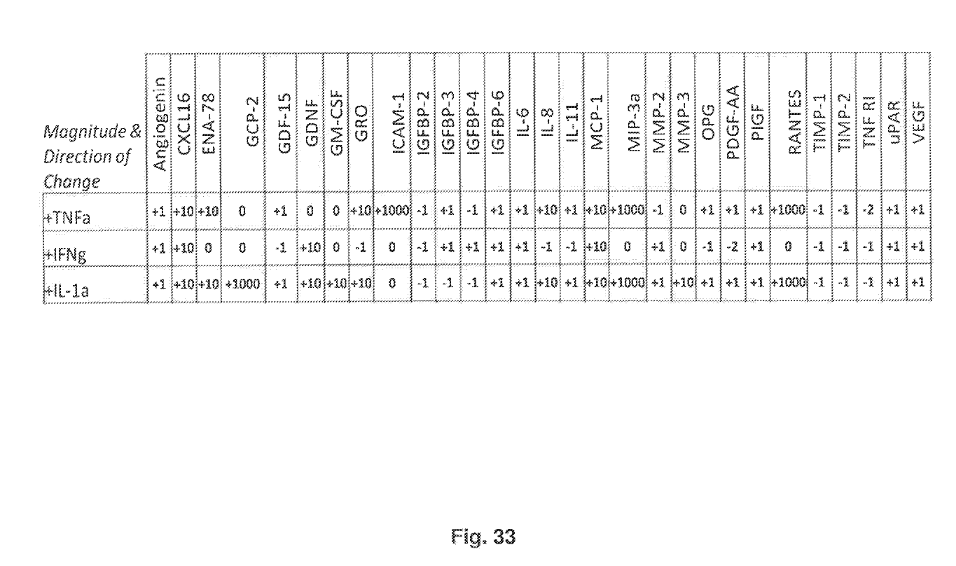

FIG. 33 provides a side-by-side comparison of the relative effects of tumor necrosis factor alpha (TNFa), interferon gamma (IFNg), and interleukin-1 alpha (IL-1a) on hABM-SC in comparison to each other. Supernatants (spnts) were collected from cultures of human exCF-SC, that were maintained for 2 days in complete media (AMEM+10% serum+glutamine) followed by 1 day in media plus vehicle (Basal) or media plus 10 ng/ml tumor necrosis factor alpha (TNFa), interferon gamma (IFNg) or interleukin-1 alpha (IL-1a). Quantitative analysis of over 150 factors present in the supernatant was completed via use of RayBiotech Inc. Quantibody arrays. The table illustrates the magnitude and direction of change, if any, when basal and treated supernatants were compared. A numeric code was used to bin the effects into 5 categories based on the magnitude and direction of effect: −2=reduction by >2; −1=reduction by 0 to −2; 0=no change; +1=induction <10; +10=induction 10 to 1000; +1000=induction >1000. These results demonstrate that human ABM-SC modify their secretion profile in response to different inflammatory markers and therefore one might expect the human ABMSC to have distinct effects depending upon the in vivo environment.

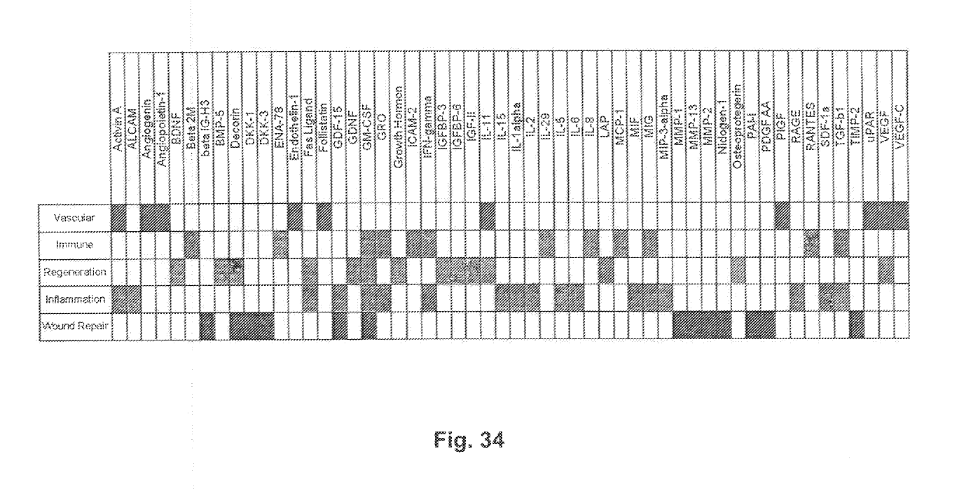

FIG. 34 portrays, without limitation, examples of various biological systems upon which induction of the indicated factors may be useful in rendering therapeutic effects (e.g., vascular, immune, regenerative, inflammatory, and wound repair systems and mechanisms).

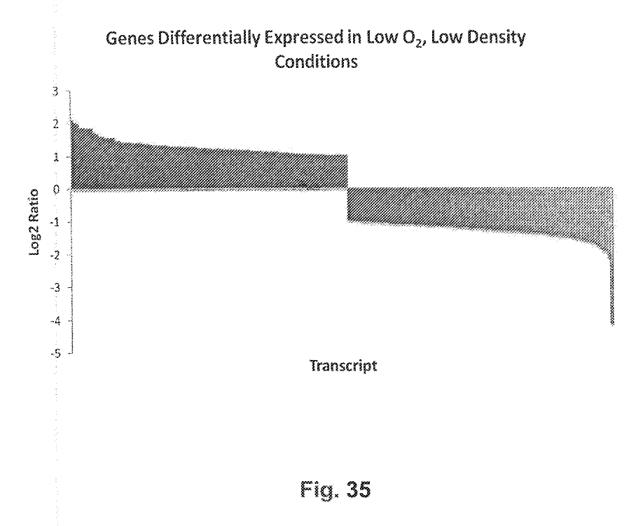

Assessment of genomic profiles of transcript expression may also be used to optimize and analyze the effects of cell culture conditions on cells and bioactive devices of the invention. For example, FIG. 35 graphically depicts the fact that nearly 200 transcripts are differentially expressed by at least at least two fold (p≦0.01) in fibroblasts grown at 4% oxygen and seeded at 30 cells/cm² vs. fibroblasts grown at 20% oxygen and seeded at 3000 cells/cm². These results are also further described below in Table 2.

Specifically, identification of differentially expressed genes was determined using Neonatal Human Dermal Fibroblasts (NHDF) cultured under 4% oxygen conditions and passaged at cell seeding densities of 30 cells/cm² compared to gene expression in NHDF cultured under 20% oxygen conditions and passaged at cell seeding densities of 3000 cells/cm². RNA was isolated from NHDF cells expanded in three flasks each under conditions of either low oxygen (4%) and low cell seeding density (30 cells/cm²) or high oxygen (20%) and high cell seeding density (3000 cells/cm²) after approximately 37 population doublings. The RNA was labeled with Cy5 and hybridized to the Human Whole Genome ONEARRAY™ from Phalanx Biotech Group (Palo Alto, Calif., USA) which contains 30,968 human probes. Fold changes for gene expression under each growth conditions were determined for all three triplicate samples. 196 probes were identified which were differentially expressed at least two fold (P≦0.01). This data demonstrates NHDFs expanded under low oxygen and low cell seeding density conditions results in a significantly different gene expression profile compared to standard tissue culture conditions.

TABLE 2
Number of Gene Transcripts Affected by Low Oxygen/Low Cell
Seeding Density Conditions

| | Number of | | Number of |
|---|---|---|---|
| | Genes | | Genes on |
| Category | Affected | P-value | Microarray |
| cell cycle process | 36 | 5.90E−19 | 544 |
| cell cycle | 31 | 5.29E−17 | 445 |
| cell division | 26 | 6.88E−17 | 291 |
| organelle organization | 36 | 1.05E−07 | 1336 |
| establishment of organelle | 7 | 1.78E−06 | 39 |
| localization | | | |
| microtubule-based process | 13 | 2.21E−05 | 262 |
| extracellular matrix | 14 | 4.88E−05 | 332 |
| DNA packaging | 5 | 0.0003 | 34 |
| DNA conformation change | 6 | 0.0011 | 75 |
| anatomical structure | 17 | 0.0029 | 688 |
| morphogenesis | | | |

### Regenerative and Therapeutic Powders

Embodiments of the invention include generation of tissues in vitro (e.g., skeletal muscle, smooth muscle, dermal, cartilaginous, etc.) using a combination of CF-SC and/or exCF-SC (from human or non-human sources) and a biodegradable matrix (e.g., collagen, PGA, etc.).

Embodiments of the invention include generation of dried or lyophilized regenerative and therapeutic powders produced from CF-SC and/or exCF-SC. Embodiments of the invention also include regenerative and therapeutic powders produced from artificial tissues and biologically compatible matrices (e.g., collagen matrices) in which CF-SC and/or exCF-SC (or components of CF-SC and/or exCF-SC) have been incorporated. For example, CF-SC and exCF-SC, CF-SC and exCF-SC incorporated into biologically compatible matrices, as well as CF-SC and exCF-SC incorporated into artificial tissues may be processed and utilized according to methods described and further referenced in U.S. Pat. No. 7,358,284 (Griffey, et al.; hereby incorporated by reference herein). Embodiments of the present invention encompass dried or lyophilized regenerative and therapeutic powders comprising CF-SC and/or exCF-SC (or components derived therefrom) which do not include or comprise a basement membrane as part of an acellular tissue matrix.

Embodiments of the invention include treating a medical condition in a patient in need of treatment by contacting a powder of the present invention with the patient. In embodiments, the regenerative and therapeutic powders of the invention are used to treat open wounds, to aid in or effect periodontal repair, for treatment of dermal deformities (e.g., acne scars, nasolabial folds), for treatment of vocal cord scars, third degree burns (e.g., applied post-debridement of dead skin, but prior to skin flap transplantation), and/or in any clinical scenario wherein the desired outcome is faster healing with less scarring. Embodiments of the invention include creating tissue de novo as described herein and subsequently converting these tissues into non-living, particulate powders that can be used as therapeutics.

Embodiments of the invention also include use of powders as a source of ECM (extracellular matrix) to construct other desired tissues.

Embodiments of the invention further include preparation and use of powders in liquid, semi-liquid, or in dry forms for application by injection, spraying, layering, packing, and in-casing in vivo in human or non-human animals.

### Veterinary Applications

Embodiments of the invention also include cells, cellular compositions, and bioactive devices derived (at least in part) from non-human cells as well as methods useful in veterinary (i.e., non-human) applications. For example, compositions and bioactive devices may be derived from, or used to treat, animals in the categories of, but without limitation to, equine (e.g., horses/donkeys), porcine (e.g., pigs), canine (e.g., dogs), feline (e.g., cats), bovine (e.g., cows), ovine (e.g., sheep), caprine (e.g., goats), camelids (e.g., camels/lamas), and murine (e.g., rats/mice).

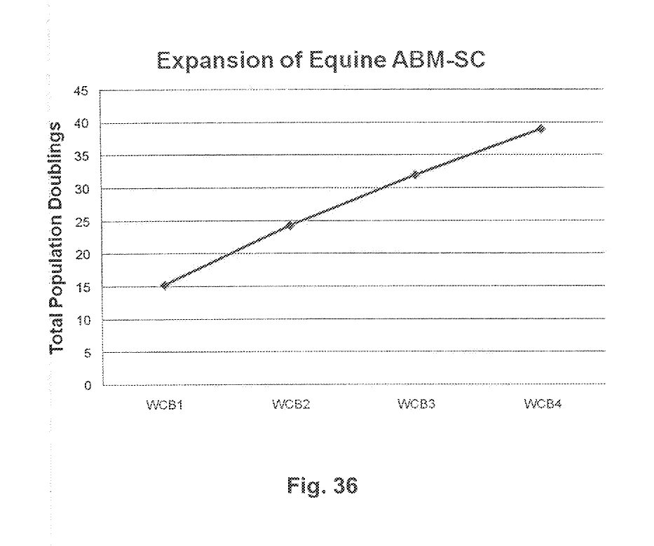

By way of example, but without limitation, it has been demonstrated that adult bone marrow derived cells from equine (horse) sources are capable of rapid proliferation and high numbers of cell doublings when cultured in vitro under low oxygen (4%) and low cell seeding (60 cells/cm²). See, FIG. 36.

In particular, FIG. 36 depicts a growth kinetic plot of equine (horse) bone marrow derived somatic cells. Bone marrow derived cells from the humerus and femur of a one month old foal were seeded at 60,000 cells/cm² and expanded at 4% oxygen to create a Master Cell Bank (MCB). The MCB and all subsequent Working Cell Banks (WCB1-WCB3) were seeded at 60 cells/cm² to determine the growth kinetics of equine ABMSCs at 4% oxygen and low cell seeding density. A total of 39 cell population doublings from the MCB was achieved over four expansions with an average of 8 cell population doublings per expansion. These results demonstrate that equine ABMSCs can be expanded with similar doublings and growth kinetics as human ABMSCs propagated under low oxygen and low cell seeding density conditions.

Additionally, adult bone marrow-derived equine (horse) cell populations cultured under the above-described conditions have been shown to exhibit the unique protein expression profile shown in Table 3. In particular, horse ABM-SCs were characterized by flow cytometry for the expression of surface markers in the master and working cell banks. Horse peripheral blood mononuclear cells (PBMCs) were used as a positive control for several markers that were negative on the horse ABMSCs. These results illustrate that horse ABMSCs can be identified by the surface markers CD44, CD49d, CD49e, CD49f, CD90, and CD147. These markers demonstrate consistent expression across all expansions. Furthermore, horse ABMSCs express GM-CSF and Vimentin across all expansions. Other surface markers that are expressed at lower percentages are CD13 and MHC I. Markers that are expressed on PBMCs but not expressed on horse ABMSCs and can be used to detect possible contaminants are CD31, CD33, CD34 and CD11b.

TABLE 3

| Marker | Description | EQ-100 | EQ-101 | EQ-102 | EQ-103 | EQ-104 | PBMC |
|---|---|---|---|---|---|---|---|
| CD11b | aM Integrin—Gran, | 9.8% | 0.1% | 0.2% | 1.1% | 0.6% | 75.0% |
| | Mono, NK | | | | | | |
| CD13 | Aminopeptidase | 68.3% | ND | ND | ND | ND | ND |
| | N—Gran, Mono | | | | | | |
| CD147 | Neurothelin—Mono, | 93.5% | ND | ND | ND | ND | ND |
| | Plate, Endo, Eryth, T sub | | | | | | |
| CD31 | PECAM-1—Platelets, | 8.5% | 2.8% | 2.6% | 3.7% | 0.6% | 39.1% |
| | Mono, Gran, B cells | | | | | | |
| CD33 | Transmembrane | 4.5% | 4.6% | 0.2% | 5.4% | 7.4% | 38.7% |
| | glycoprotein—Myloid | | | | | | |
| | progenitors, Mono | | | | | | |
| CD34 | Transmembrane | 2.9% | 2.6% | 0.8% | 4.6% | 1.4% | 20.7% |
| | glycoprotein—Hemopoietic | | | | | | |
| | progenitors | | | | | | |
| CD44 | HCAM—Leukocytes, | 99.9% | 99.8% | 99.8% | 99.8% | 99.6% | ND |
| | Erythrocytes | | | | | | |
| CD49d | a4 Integrin—Lympho, | 99.8% | 99.8% | 96.5% | 99.4% | 99.6% | ND |
| | Mono, Esino | | | | | | |
| CD49e | a5 Integrin—Mono, | 95.9% | 91.5% | 93.4% | 96.6% | 97.4% | ND |
| | Platelets | | | | | | |
| CD49f | a6 Integrin—Mono | 99.5% | 95.7% | 99.1% | 99.5% | 99.1% | ND |
| CD90 | Thy-1—Thymic stromal, | 87.2% | 100.0% | 100.0% | 100.0% | 99.8% | ND |
| | hematopoietic prog. | | | | | | |
| GM-CSF | | 97.1% | 97.1% | 99.1% | 99.5% | 97.3% | ND |
| MHC I | All nucleated cells | 22.6% | 8.5% | 6.2% | 8.6% | 12.9% | 97.7% |
| Vimentin | Mesoderm derived cells | 98.7% | 99.4% | 97.7% | 99.0% | 99.4% | ND |

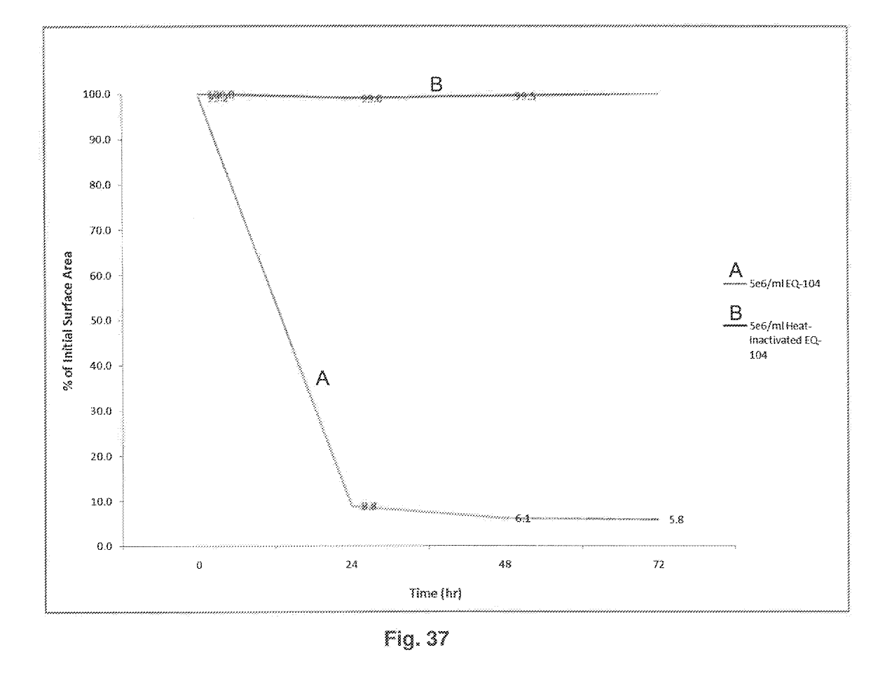

It has also been demonstrated that adult bone marrow-derived equine (horse) cell populations exhibit the same type of bioactivity (gel contraction) when cultured in a collagen matrix as has been previously demonstrated for human and porcine adult bone marrow-derived cells. See, e.g., FIG. 37. For example, FIG. 37 shows results obtained when equine ABM-SCs were seeded at 5e6 cells/ml within 1.6 mg/ml rat tail collagen gels and cultured suspended in media for 3 days. Diameters of the gel constructs were measured at 24, 48, and 72 hrs. Percent surface area contraction was calculated by comparing x and y dimension initial diameters to contracted diameters of each timepoint. A control gel containing heat inactivated cells showed little contraction with the active cells contracting the gels significantly to 5.8% the initial size after 3 days in culture.

In another embodiment of the invention, hABM-SC are also capable of producing significant quantities of VEGF in biocompatible cell matrices. For example, FIG. 38 depicts VEGF levels within cultured exCF-SC-seeded Poly-Lactic-co-Glycolic Acid (PLGA) scaffolds. Cells were seeded at varying densities of 3e6 cells or 7e6 cells onto 2 cm diameter, non-woven PLGA polymer scaffolds and cultured for either 1, 3, or 6 days. Constructs were replenished with fresh media on culture day 2 and 4. At each timepoint, constructs were washed 3× in balanced salt solution and snap frozen. Lysates were made of each construct by mechanical dissociation in protein extraction buffer. ELISA was used on construct lysates to quantify amount (in ng) of VEGF contained within hABM-SC seeded PLGA constructs. Results indicate an increase in VEGF contained within the PLGA constructs with increasing cell density.

Embodiments of the invention include ABM-SC (for example, human or non-human CF-SC or exCF-SC) seeded into biocompatible matrices (such as a PLGA scaffold) at densities in a range of about 100 cells/mm³ to about 100000 cells/mm³. For example, embodiments of the invention include cells seeded into biocompatible matrices at about 100, 500, 1000, 2000, 3000, 4000, 5000, 6000, 7000, 8000, 10000, 15000, 20000, 25000, 30000, 35000, 40000, 45000, 50000, 60000, 70000, 80000, 90000 and 100000 cells/mm³ or greater.

### Additional Regenerative and Therapeutic Compositions and Applications

Embodiments of the invention include use of bioactive compositions of the invention for treatment and management of acute and chronic trauma-related injuries (e.g., such as occur among military personnel in combat or in other individuals suffering burns or injuries inflicted by high velocity projectiles). Accordingly, embodiments of the invention include methods and compositions useful for treatment and repair of burn injuries, dermal wounds, traumatic brain injury. Embodiments of the invention include use of compositions of the invention for treatment and preservation of function of nerve cells and neuronal signaling (including, for example, but not limited to, treatment of neuropathological disease conditions such as Parkinson's and Alzheimer's Disease).

Embodiments of the invention also include use of compositions of the invention for treatment and repair of surgically induced injuries such as in patients undergoing mastectomy/breast reconstructive surgery to promote healing and reduction of scarring at the incisional site.

Additional embodiments of the invention include use of CF-SC and/or exCF-SC (or components derived therefrom) either directly, or as incorporated into biocompatible matrices, as incorporated into bioactive compositions and devices, and as incorporated into regenerative and therapeutic powders (which may be applied in any final form whether as a dry powder, liquid, semi-liquid, paste, solid, or semi-solid) wherein said uses may particularly encompass (without limitation):

Use of CF-SC and/or exCF-SC (or components derived therefrom) incorporated into biocompatible sheets (i.e., sheet-like matrices; which may be packaged into packets for mobility and rapid application on burns or other wounds “in the field”);

Use of CF-SC and/or exCF-SC (or components derived therefrom) in suspension (e.g., for example, for application by spraying, pouring, or pasting on open wounds);

Use of CF-SC and/or exCF-SC (or components derived therefrom) for mitigation of injuries such as traumatic brain injury;

Use of CF-SC and/or exCF-SC (or components derived therefrom) to prevent or mitigate sepsis;

Use of CF-SC and/or exCF-SC (or components derived therefrom) to promote or effect healing of subacute injuries (e.g., to improve limb salvage and prevent amputation);

Use of CF-SC and/or exCF-SC (or components derived therefrom) to improve autologous skin graft outcomes (e.g., to reduce rejection risk or “failure to take”);

Use of CF-SC and/or exCF-SC (or components derived therefrom) for reduction or revision of disfiguring scars;

Use of CF-SC and/or exCF-SC (or components derived therefrom) to slow the course of Parkinson's Disease, Alzheimer's or other neurological pathologies;

Use of CF-SC and/or exCF-SC (or components derived therefrom) to aid in healing chronic and poorly healing wounds; and

Use of CF-SC and/or exCF-SC (or components derived therefrom) to reduce, improve, or remove restrictive scars (e.g., to increase mobility and quality of life).

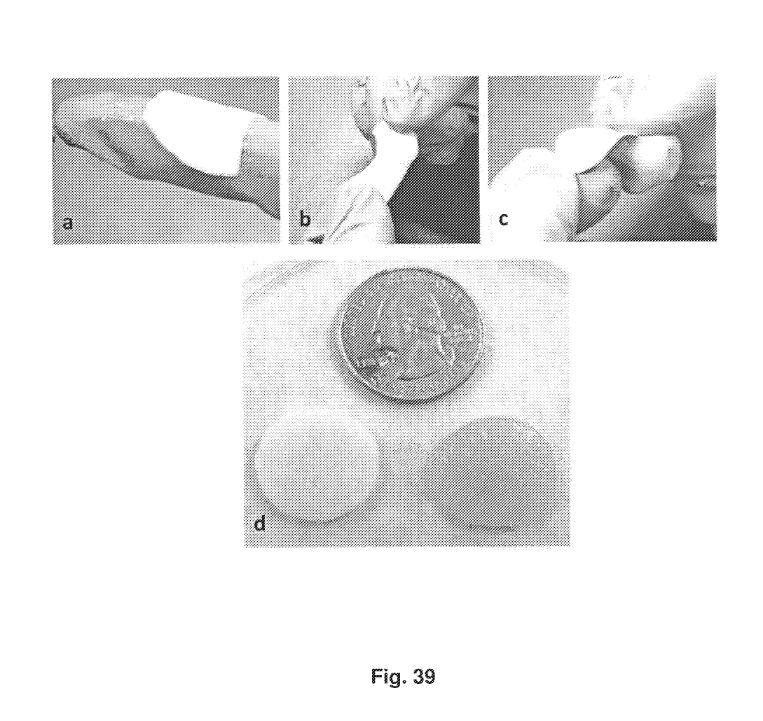

In certain embodiments of the invention, ABM-SC are useful in tissue engineered constructs of various shapes and forms. For example, FIG. 39 shows photographs of: A & B) hABM-SC seeded porcine collagen gel after culture and cross-linking to generate a non-living mechanically stable bioactive construct; C) hABM-SC seeded porcine collagen gel after culture and dehydration to generate a non-living thin film bioactive construct; and D) non-woven PLGA scaffold (left) and hABM-SC seeded non-woven PLGA scaffold cultured construct

Additional embodiments of the invention also include:

Bioactive compositions and devices wherein CF-SC and/or exCF-SC seeded scaffolds are co-cultured with other cell types to allow stimulation of tissue specific factors from CF-SC and/or exCF-SC (e.g., for production of factors useful in treatment of neural, vascular, bone, cartilage, cardiac condition);

Compositions and devices wherein CF-SC and/or exCF-SC seeded scaffolds are cultivated with varied O₂, N₂ and CO₂ gas ratios to further optimize desired bioactivity (this may include varying percentages of oxygen present in the air contacting the cultured constructs to produce hypoxic, atmospheric, or hyperoxic conditions);

Compositions and devices wherein CF-SC and/or exCF-SC seeded scaffolds are cultivated under varied culture media conditions to further optimize desired bioactivity (this may include variation of additions of chemical factors such as growth factor proteins, vitamins, minerals, amino acids, sugars, fatty acids, and buffers);

Compositions and devices wherein particulate forms of natural matrix or synthetic polymers are seeded with CF-SC and/or exCF-SC, or CF-SC and/or exCF-SC are encapsulated into particulate forms, for microcarrier delivery vehicles of cells (or components derived therefrom) to the patient. Further, the particulate forms of tissue engineered constructs generated in vitro with CF-SC and/or exCF-SC and scaffolds are used in combination with viable CF-SC and/or exCF-SC for carrier delivery vehicles into the patient.

Tissue engineered constructs generated from CF-SC and/or exCF-SC and scaffolds may be used to produce bioactive films, bandages, patches, sutures, meshes, or wraps. Multiple shapes, sizes, and thicknesses of these constructs can be designed for specific applications. Bandages or patches can be applied to cover damaged skin tissue. Flexible constructs can be used as bioactive wraps to enclose more irregularly shaped tissues such as bone, ligaments, tendon, nerve, and muscle.

Arrangements of CF-SC and/or exCF-SC and matrix scaffolds in culture can be specifically designed for different construct preparations including cell encapsulation, cell seeding around outside of scaffold, cell-scaffold juxtaposition for secretion of factors from cells into scaffold.

### Immune Disorders

Cells and compositions of the present invention may be used to prevent, treat, and/or ameliorate, inter alia, immune, autoimmune, and inflammatory diseases and disorders. Some examples of such disorders are indicated below; these lists are exemplary only and are not intended to be comprehensive with respect to all immune, autoimmune, and inflammatory diseases and disorders; nor should the following be construed as limiting with respect to pathologies which may be treated with the cells and compositions of the present invention.

Example of some diseases with a complete or partial autoimmune etiology: Acute disseminated encephalomyelitis (ADEM), Addison's disease, Ankylosing spondylitis, Antiphospholipid antibody syndrome (APS), Aplastic anemia, Autoimmune hepatitis, Autoimmune Oophoritis, Celiac disease, Crohn's disease, Diabetes mellitus type 1, Gestational pemphigoid, Goodpasture's syndrome, Graves' disease, Guillain-Barré syndrome (GBS), Hashimoto's disease, Idiopathic thrombocytopenic purpura, Kawasaki's Disease, Lupus erythematosus, Multiple sclerosis, Myasthenia gravis, Opsoclonus myoclonus syndrome (OMS), Optic neuritis, Ord's thyroiditis, Pemphigus, Pernicious anaemia, Polyarthritis, Primary biliary cirrhosis, Rheumatoid arthritis, Reiter's syndrome, Sjögren's syndrome, Takayasu's arteritis, Temporal arteritis (also known as “giant cell arteritis”), Warm autoimmune hemolytic anemia, and Wegener's granulomatosis.

Examples of some diseases suspected of being linked to autoimmunity: Alopecia universalis, Behcet's disease, Chagas' disease, Chronic fatigue syndrome, Dysautonomia, Endometriosis, Hidradenitis suppurativa, Interstitial cystitis, Lyme disease, Morphea, Neuromyotonia, Narcolepsy, Psoriasis, Sarcoidosis, Scleroderma, Ulcerative colitis, Vitiligo, and Vulvodynia.

Examples of some immune hypersensitivity diseases and disorders: Allergic asthma, Allergic conjunctivitis, Allergic rhinitis (“hay fever”), Anaphylaxis, Myasthenia gravis., Angioedema, Arthus reaction, Atopic dermatitis (eczema), Autoimmune hemolytic anemia, Autoimmune Pernicious anemia, Coeliac disease, Contact dermatitis (poison ivy rash, Eosinophilia, Erythroblastosis Fetalis, Farmer's Lung (Arthus-type reaction), for example), Goodpasture's syndrome, Graves' disease, Graves' disease, Hashimoto's thyroiditis, Hemolytic disease of the newborn, Immune complex glomerulonephritis, Immune thrombocytopenia, Myasthenia gravis, Pemphigus, Rheumatic fever, Rheumatoid arthritis, Serum sickness, Subacute bacterial endocarditis, Symptoms of leprosy, Symptoms of malaria, Symptoms of tuberculosis, Systemic lupus erythematosus, Temporal arteritis, Transfusion reactions, Transplant rejection, and Urticaria (hives).

Example of some inflammatory disorders: allergies, ankylosing spondylitis, arthritis, asthma, autistic enterocolitis, autoimmune diseases, Behcet's disease, chronic inflammation, glomerulonephritis, inflammatory bowel disease (IBD), inflammatory bowel diseases, pelvic inflammatory disease, psoriasis, psoriatic arthritis, reperfusion injury, rheumatoid arthritis, transplant rejection, and vasculitis.

Example of some immunodeficiency disorders: B cell deficiencies (such as X-linked agammaglobulinemia and Selective Immunoglobulin Deficiency), T cell deficiencies (such as DiGeorge's syndrome (Thymic aplasia), Chronic mucocutaneous candidiasis, Hyper-IgM syndrome and, Interleukin-12 receptor deficiency), Combined T cell and B cell abnormalities (such as Severe Combined Immunodeficiency Disease (SCID), Wiskott-Aldrich syndrome, and Ataxia-telangiectasia), Complement Deficiencies (such as Hereditary Angioedema or Hereditary angioneurotic edema and Paroxysmal nocturnal hemoglobinuria), Phagocyte deficiencies (such as Leukocyte adhesion deficiency, Chronic Granulomatous Disease (CGD), Chédiak-Higashi syndrome, Job's syndrome (Hyper-IgE syndrome), Cyclic neutropenia, Myeloperoxidase deficiency, Glucose-6-phosphate dehydrogenase deficiency, and Interferon-γ deficiency), and Common Variable Immunodeficiency (CVID), Vici syndrome, and Acquired immune deficiency syndrome (AIDS).

### Embodiments of the Invention

Particular embodiments of the invention include the following:

A1. A method of administering a therapeutically useful amount of a biological composition or compositions to a subject, comprising administering to said subject an isolated population of self-renewing colony forming cells, wherein the cells in said cell population have substantially no multipotent differentiation capacity, wherein said cells have a normal karyotype, and wherein said cells are non-immortalized.

A2. A method of administering a therapeutically useful amount of a biological composition or compositions to a subject, comprising:
(i) isolating the biological composition or compositions produced by an isolated population of self-renewing colony forming cells; and,
(ii) administering said biological composition or compositions to said subject,
   wherein the cells in said cell population have substantially no multipotent differentiation capacity, wherein said cells have a normal karyotype, and wherein said cells are non-immortalized.

A3. A method of repairing, treating, or promoting regeneration of damaged tissue in a subject, comprising administering to said subject an effective amount of an isolated population of self-renewing colony forming cells, wherein the cells in said cell population have substantially no multipotent differentiation capacity, wherein said cells have a normal karyotype, and wherein said cells are non-immortalized.

A4. A method of repairing, treating, or promoting regeneration of damaged tissue in a subject, comprising:
(i) isolating the biological composition or compositions produced by an isolated population of self-renewing colony forming cells; and,
(ii) administering said biological composition or compositions to said subject,
   wherein the cells in said cell population have substantially no multipotent differentiation capacity, wherein said cells have a normal karyotype, and wherein said cells are non-immortalized.

A5. A method of treating or reducing inflammation, immune, or autoimmune activity in a subject, comprising administering to said subject an effective amount of an isolated population of self-renewing colony forming cells, wherein the cells in said cell population have substantially no multipotent differentiation capacity, wherein said cells have a normal karyotype, and wherein said cells are non-immortalized.

A6. A method of treating or reducing inflammation, immune, or autoimmune activity in a subject, comprising:
(i) isolating the biological composition or compositions produced by an isolated population of self-renewing colony forming cells; and,
(ii) administering said biological composition or compositions to said subject,
   wherein the cells in said cell population have substantially no multipotent differentiation capacity, wherein said cells have a normal karyotype, and wherein said cells are non-immortalized.

A7. The method of any of embodiments A1 to A6, wherein prior to administration, said cell population has been passaged in vitro for a number of population doublings sufficient to cause the cells in said population to lose multipotent differentiation capacity.

A8. The method of any one of embodiments A1 to A7, wherein said cell population has unipotent differentiation capacity.

A9. The method of any of embodiments A1 to A8, wherein said cells have substantial capacity for self-renewal.

A10. The method of any of embodiments A1 to A9, wherein prior to administration said cell population has been passaged in vitro for a number of population doublings while retaining substantial capacity for self-renewal.

A11. The method of any one of embodiments A1 to A10, wherein the cells in said isolated cell population are not embryonic stem cells.

A12. The method of any one of embodiments A1 to A11, wherein the cells in said isolated cell population are not stem cells, mesenchymal stem cells, hematopoietic stem cells, multipotent adult progenitor cells (MAPCs), multipotent adult stem cells (MASCs), or fibroblasts.

A13. The method of any one of embodiments A1 to A12, wherein said cells do not differentiate into one or more cell types selected from the group consisting of:

a) osteocytes; b) adipocytes; and, c) chondrocytes.

A14. The method of any one of embodiments A1 to A13, wherein said cells do not deposit detectable levels of calcium following treatment under osteoinductive conditions.

A15. The method of embodiment A14, wherein said osteoinductive conditions include exposure to exogenously supplied Noggin.

A16. The method of any one of embodiments A1 to A15, wherein the cells in said isolated cell population are derived from connective tissue.

A17. The method of any one of embodiments A1 to A16, wherein the cells in said isolated cell population are stromal cells.

A18. The method of any one of embodiments A1 to A17, wherein the cells in said isolated cell population co-express CD49c and CD90.

A19. The method of any one of embodiments A1 to A18, wherein the cell population maintains an approximately constant doubling rate through multiple in vitro cell doublings,

A20. The method of any one of embodiments A1 to A19, wherein said cells are negative for detectable expression of one or more antigens selected from the group consisting of:

a) CD10; b) STRO-1; and, c) CD106/V/CAM-1.

A21. The method of any one of embodiments A1 to A20, wherein said cells are positive for detectable expression of one or more antigens selected from the group consisting of:

a) CD44; b) HLA Class-1 antigen; and, c) (beta) 2-Microglobulin,

A22. The method of any one of embodiments A1 to A21, wherein said cells express or secrete detectable quantities of compositions selected from the group consisting of:

a) TNF-RI; b) soluble TNF-RI; c) TNF-RII; d) soluble TNF-RII; e) IL-1R antagonist;

and, f) IL-18 binding protein.

A23. The method of any one of embodiments A1 to A21, wherein said cells express or secrete detectable quantities of compositions selected from the group consisting of compositions shown in Table 1A, 1B and 1C.

A24. The method of any one of embodiments A1 to A23, wherein the cells in said isolated cell population are initially isolated from a tissue source selected from the group consisting of:

a) bone marrow; b) adipose tissue/fat; c) skin; d) placental; e) umbilical cord; f) tendon; g) ligament; h) muscle fascia; and, i) other connective tissues.

A25. The method of embodiment A24, wherein said tissue source is human.

A26. The method of any one of embodiments A1 to A25, wherein said cell population maintains an approximately constant doubling rate through a number of in vitro cell doublings selected from the group consisting of:

a) 1 to 5 cell doublings; b) 5 to 10 cell doublings; c) 10 to 20 cell doublings; d) 20 to 30 cell doublings; e) 30 to 40 cell doublings; f) 40 to 50 cell doublings; g) 1 to 50 cell doublings; h) 5 to 50 cell doublings; i) 10 to 50 cell doublings; j) 20 to 50 cell doublings; k) 30 to 50 cell doublings; l) 1 to 10 cell doublings; m) 1 to 20 cell doublings; n) 1 to 30 cell doublings; o) 1 to 40 cell doublings; p) 5 to 20 cell doublings; q) 5 to 30 cell doublings; r) 5 to 40 cell doublings; s) 10 to 30 cell doublings; t) 10 to 40 cell doublings; and, u) 20 to 40 cell doublings.

A27. The method of any one of embodiments A1 to A26, wherein said cell population has undergone a number of population doublings selected from the group consisting of:

a) at least about 10 population doublings; b) at least about 15 population doublings; c) at least about 20 population doublings; d) at least about 25 population doublings; e) at least about 30 population doublings; f) at least about 35 population doublings; g) at least about 40 population doublings; h) at least about 45 population doublings; and, i) at least about 50 population doublings.

A28. The method of any one of embodiments A1 to A27, wherein said biological composition or compositions are bound in or to the cell surface of said cell populations.

A29. The method of any one of embodiments A1 to A28, wherein said biological composition or compositions are secreted into the extracellular environment of said cell populations.

A30. The method of any one of embodiments A1 to A29, wherein said biological composition or compositions are one or more molecules selected from the group consisting of:

a) proteins; b) carbohydrates; c) lipids; d) fatty acids; e) fatty acid derivatives; d) gases; and, e) nucleic acids.

A31. The method of embodiment A30, wherein said proteins are selected from the group consisting of:

a) glycosylated proteins; b) cytokines; c) chemokines; d) lymphokines; e) growth factors; f) trophic factors, g) morphogenetic proteins; and, h) hormones.

A32. The method of embodiment A31, wherein said wherein said biological composition or compositions bind to and inactivate, or reduce, the biological activity of molecules selected from the group consisting of:

a) fatty acids; b) fatty acid derivatives; c) receptor molecules; d) cytokines; e) chemokines; lymphokines; g) growth factors; h) trophic factors, i) morphogenetic proteins; and, j) hormones.

A33. The method of embodiment A32, wherein said biological composition or compositions are soluble receptors that bind cognate ligands selected from the group consisting of:

a) fatty acids; b) fatty acid derivatives; c) receptor molecules; d) cytokines; e) chemokines; f) lymphokines; g) growth factors; h) trophic factors, i) morphogenetic proteins; and, j) hormones.

A34. The method of any one of embodiments A1 to A33, wherein said cells are induced to increase expression of one or more biological compositions.

A35. The method of any one of embodiments A1 to A33, wherein said cells are induced to express one or more biological compositions.

A36. The method of any one of embodiments A1 to A29, wherein said one or more biological compositions is/are selected from Table 1A, 1B and 1C.

A37. The method of any one of embodiments A1 to A29, wherein said one or more biological compositions is selected from the group consisting of:

a) TNF-RI; b) soluble TNF-RI; c) TNF-RII; d) soluble TNF-RII; e) IL-1R antagonist; and, f) IL-18 binding protein.

A38. The method of any one of embodiments A1 to A37, wherein the cells in said cell population du not exhibit long-term engraftment in, or with, tissues or organs when administered to a living mammalian organism.

A39. The method of any one of embodiments A1 to A38, wherein the cells in said cell population maintain approximately constant levels of production of one or more therapeutically useful compositions in vivo.

A40. The method of embodiment A39, wherein said levels of production are maintained for a measure of time selected from the group consisting of:

a) at least about 24 hours; b) at least about 48 hours; c) at least about 72 hours; d) at least about 4 days; e) at least about 5 days; f) at least about 6 days; g) at least about 7 days; h) at least about 2 weeks; i) at least about 3 weeks; j) at least about 4 weeks; k) at least about 1 month; 1) at least about 2 months; m) at least about 3 months; n) at least about 6 months; and, o) at least about 1 year.

A41. The method of any one of embodiments A1 to A40, wherein said patient is human.

A42. The method of any one of embodiments A1 to A41, wherein said method is used to treat a disease or disorder selected from the group consisting of:

a) a neurological disease or disorder; b) a cardiac disease or disorder; c) a skin disease or disorder; d) a skeletal muscle disease or disorder; e) a respiratory disease or disorder; f) a hepatic disease or disorder; g) a renal disease or disorder; h) a genitourinary system disease or disorder; i) a bladder disease or disorder; j) an endocrine disease or disorder; k) a hematopoietic disease or disorder; l) a pancreatic disease or disorder; m) diabetes; n) an ocular disease or disorder; o) a retinal disease or disorder; p) a gastrointestinal disease or disorder; q) a splenic disease or disorder; r) an immunological disease or disorder; s) an autoimmune disease or disorder; t) an inflammatory disease or disorder; u) a hyperproliferative disease or disorder; and, v) cancer.

A43. The method of any one of embodiments A1 to A42, wherein said cells are genetically modified.

A44. The method of embodiment A43, wherein said cells are genetically modified by introduction of a recombinant nucleic acid molecule.

A45. A process for making an isolated cell population in any one of embodiments A1 to A47, wherein said process comprises:

i) obtaining a source population of cells from an organism; and,

ii) culturing said source population of cells in vitro.

B1. A composition comprising a pharmaceutically acceptable mixture of self-renewing, colony-forming somatic cells (CF-SC), or conditioned cell culture media derived from such cells, and purified naturally occurring or isolated recombinant extracellular matrix or blood plasma proteins.

B2. The composition of embodiment B1, wherein said CF-SC are derived from bone marrow.

B3. The composition of embodiments B1 or B2, wherein said CF-SC are derived from a human.

B4. The composition of any one of embodiments B1-B3, wherein said CF-SC are derived from an adult mammal, including humans.

B5. The composition of any one of embodiments B1-B4, wherein said CF-SC express one or more secreted proteins shown in Table 1A, 1B and 1C.

B6. The composition of any one of embodiment B1-B5, wherein said extracellular matrix or blood plasma proteins comprise one or more full-length or alternatively processed isoforms, proteolytic fragments, or subunits of molecules selected from the group consisting of:

a) collagen; b) elastin; c) fibronectin; d) laminin; e) entactin (nidogen); f) hyaluronic acid; g) polyglycolic acid (PGA); h) fibrinogen (Factor I); i) fibrin; j) prothrombin (Factor II); k) thrombin; 1) anti-thrombin; m) Tissue factor Co-factor of VIIa (Factor III); n) Protein C; o) Protein S; p) protein Z; q) Protein Z-related protease inhibitor; r) heparin cofactor II; s) Factor V (proaccelerin, labile factor); t) Factor-VII; u) Factor-VIII; v) Factor-IX; w) Factor-X; x) Factor-XI; y) Factor-XII; z) Factor-XIII; aa) von Willebrand factor; ab) prekallikrein; ac) high molecular weight kininogen; ad) plasminogen; ae) plasmin; af) tissue-plasminogen activator; ag) urokinase; ah) plasminogen activator inhibitor-1; and, ai) plasminogen activator inhibitor-2.

B7. The composition of any one of embodiments B1-B6, further comprising purified naturally occurring or isolated recombinant cytokines or chemokines.

B8. The composition of any one of embodiments B1-B7, wherein said extracellular matrix, blood plasma proteins, cytokines, and/or chemokines are derived from humans.

B9. The composition of any one of embodiments B1-B8, wherein said pharmaceutically acceptable mixture forms a semi-solidified or solidified matrix.

B10. A method of treating damaged tissue with the composition of any one of embodiments B1-B8, wherein the composition is a liquid.

B11. The method of embodiment B10, wherein the liquid is applied by injection.

B12. A method of treating damaged tissue with the composition of any one of embodiments 1-9, wherein the composition is applied as a liquid but thereafter forms a semi-solidified or solidified matrix.

B13. The method of embodiments B10-B12 wherein said tissue is damaged as a result of a condition selected from the group consisting of:

a) disease; b) physical trauma; c) ischemia; d) aging; e) burn; f) bacterial infection; g) viral infection; h) fungal infection; and, i) dysregulation of the immune system.

B14. The method of embodiment B13, wherein the damaged tissue is skin.

B15. A method of using the composition of any one of embodiments B1-B9 for facial skin rejuvenation.

B16. A method of using the composition of any one of embodiments B1-B9, wherein said composition inhibits acute inflammation.

C1. A method for treating, repairing, regenerating, or healing a damaged organ or tissue comprising contacting said damaged organ or tissue with an effective amount of self-renewing colony forming somatic cells or compositions produced from such cells so as to effect said treatment, repair, regeneration, or healing of the damaged organ or tissue.

C2. The method of embodiment C1, wherein said damaged organ or tissue is contacted with an effective amount of self-renewing colony forming somatic cells or compositions produced from such cells by means selected from the group consisting of:

a) injection into the damaged organ or tissue; b) application onto the damaged organ or tissue; c) injection proximal to the damaged organ or tissue; d) application proximal to the damaged organ or tissue; and, e) intravenous administration.

C3. The method of embodiments C1 or C2, wherein the cells are derived from bone marrow.

C4. The method of any one of embodiments C1-C3, wherein the cells are human.

C5. The method of any one of embodiments C₁-C₄, wherein the cells, or compositions produced by said cells, inhibit or reduce adverse immune responses (such as cell-mediated autoimmunity), fibrosis (scarring) and/or adverse tissue remodeling (for example, ventricular remodeling).

C6. The method of any one of embodiments C1-C5, wherein the cells, or compositions produced by said cells, control inflammation and/or inhibit acute inflammation.

C7. The method of any one of embodiments C1-C5, wherein the cells, or compositions produced by said cells, stimulate or enhance angiogenesis.

C8. The method of any one of embodiments C1-C5, wherein said cells do not exhibit significant or detectable levels of permanent or long-term engraftment into said damaged organs or tissues.

C9. The method of any one of embodiments C1-C8, wherein said damaged organs are selected from the group consisting of heart, brain, and spinal cord.

C10. The method of any one of embodiments C1-C8, wherein said damaged tissue is selected from the group consisting of cardiac tissue, neuronal tissue (including central and peripheral nervous system tissue), and vascular tissue (including major and minor arteries, veins, and capillaries).

D1. A method of inducing, enhancing, and/or maintaining the generation of new red blood cells in vitro.

D2. The method of embodiment D1, wherein said induction, enhancement, or maintenance is achieved by co-cultivation of hematopoietic precursor cells with self-renewing colony forming cells.

D3. The method of embodiment D2, wherein said self-renewing colony forming cells are human bone marrow-derived somatic cells (hABM-SC).

D4. The method of embodiment D3, wherein said hABM-SC are derived from an adult.

D5. The method of any one of embodiments D1-D3, wherein said co-cultivation utilizes a semi-permeable barrier to maintain separation of the hematopoietic precursor cells from the self-renewing colony forming cells while allowing exchange of compositions produced by said self-renewing colony forming cells across said barrier.

D6. The method of embodiment D1, wherein said induction, enhancement, or maintenance is achieved by co-cultivation of hematopoietic precursor cells with isolated compositions produced by self-renewing colony forming cells.

D7. The method of embodiment D5, wherein said self-renewing colony forming cells are human bone marrow-derived somatic cells (hABM-SC).

D8. The method of embodiment D6, wherein said hABM-SC are derived from an adult.

D9. The method of any one of embodiments D5-D7, wherein said isolated compositions are lyophilized.

D10. The method of any one of embodiments D5-D7, wherein said isolated compositions are cryopreserved.

D11. The method of any one of embodiments D5-D7, wherein said isolated compositions are mixed with one or more pharmaceutically acceptable carriers.

D12. A method of producing, isolating, purifying, and/or packaging cell-derived compositions and/or trophic factors.

D13. A method of producing conditioned media, wherein said media contains sera or is sera-free media.

D14. A method of isolating and purifying fractions and/or cell-derived compositions from conditioned media, wherein said media contains sera or is sera-free media.

D15. A method of isolating, cryopreserving, and/or expanding CD34+Cord

Blood Cells (CBC).

D16. The method of embodiment D15, wherein said CBC are expanded in suspension cultures.

D17. The method of embodiment D15, wherein said CBC are expanded by co-culturing with a feeder layer of self-renewing colony forming cells.

D18. The method of embodiment D17, wherein said self-renewing colony forming cells are human bone marrow-derived somatic cells (hABM-SC).

D19. A wash solution comprising Balanced Salt Solution with dextrose (BSSD).

D20. The wash solution of embodiment D19 wherein said dextrose is at a concentration of about 4.5% dextrose.

D21. The wash solution of embodiment D19 or D20, further comprising human serum albumin.

D22. The wash solution of embodiment D21, wherein said human serum albumin is at a concentration of about 5% human serum album.

D23. A cryopreservation media comprising dimethyl sulfoxide (DMSO) and human serum albumin in a Balanced Salt Solution.

D24. The cryopreservation media of embodiment D23, wherein said DMSO concentration is about 5% and said HSA concentration is about 5%.

E1. An isolated cell population derived from bone marrow, wherein greater than about 91% of the cells of the cell population co-express CD49c and CD90, and wherein the cell population has a doubling rate of less than about 30 hours.

E2. The isolated cell population of embodiment E1, wherein the cell population is derived from human bone marrow.

E3. The isolated cell population of embodiments E1 or E2, wherein the cells of the cell population that co-express CD49c and CD90 do not express CD34 and/or CD45.

E4. The isolated cell population according to any one of embodiments E1, E2, or E3, wherein the cells of the cell population that co-express CD49c and CD90 further express at least one cardiac-related transcription factor selected from the group consisting of GATA-4, Irx4, and Nkx2.5.

E5. The isolated cell population according to any one of embodiments E1, E2, or E3, wherein the cells of the cell population that co-express CD49c and CD90 further express at least one trophic factor selected from the group consisting of:

### a) Brain-Derived Neurotrophic Factor (BDNF);

### b) Cystatin-C;

### c) Interleukin-6 (IL-6);

### d) Interleukin-7 (IL-7);

### e) Interleukin-11 (IL-11);

### f) Nerve Growth Factor (NGF);

### g) Neurotrophin-3 (NT-3);

### h) Macrophage Chemoattractant Protein-1 (MCP-1);

### i) Matrix Metalloproteinase-9 (MMP-9);

j) Stem Cell Factor (SCF); and,

### k) Vascular Endothelial Growth Factor (VEGF).

E6. The isolated cell population according to any one of embodiments E1, E2, or E3, wherein the cells of the cell population that co-express CD49c and CD90 further express p21 or p53, and wherein expression of p53 is a relative expression of up to about 3000 transcripts of p53 per 10⁶ transcripts of an 18s rRNA and expression of p21 is a relative expression of up to about 20,000 transcripts of p21 per 10⁶ transcripts of an 18s rRNA.

E7. The isolated cell population according to any one of embodiments E1, E2, or E3, wherein the isolated cell population has been cultured in vitro through a number of population doublings selected from the group consisting of:

a) at least about 15 population doublings;
b) at least about 20 population doublings;
c) at least about 25 population doublings;
d) at least about 30 population doublings;
e) at least about 35 population doublings; and,
f) at least about 40 population doublings.

E8. A method of making an isolated cell population derived from bone marrow, wherein greater than about 91% of the cells of the cell population co-express CD49c and CD90, and wherein the cell population has a doubling rate of less than about 30 hours, comprising the steps of:

a) culturing a source of the cell population under a low oxygen condition or a low oxidative stress condition to produce an adherent cell population; and,

b) culturing the adherent cell population at a seeding density of less than about 2500 cells/cm².

E9. The method of embodiment E8, wherein the cell population is derived from human bone marrow.

E10. The method of embodiments E8 or E9, wherein the source of the cell population in embodiment 8, part a) is cultured at an initial seeding density selected from the group consisting of:

a) less than about 75000 cells/cm²; and,
b) less than about 50000 cells/cm².

E11. The method of any one of embodiments E8 to E10, wherein the adherent cell population in embodiment 8, part b) is cultured at a seeding density selected from the group consisting of:

a) less than about 2500 cells/cm²;
b) less than about 1000 cells/cm²;
c) less than about 100 cells/cm²;
d) less than about 50 cells/cm²; and,
e) less than about 30 cells/cm².

E12. The method of any one of embodiments E8 to E11, wherein the low oxygen condition is selected from the group consisting of:

a) between about 1 to 10% oxygen;
b) between about 2 to 7% oxygen;
d) less than about 20% oxygen;
c) less than about 15% oxygen;
d) less than about 10% oxygen;
e) less than about 5% oxygen; and,
f) about 5% oxygen.

E13. The method of any one of embodiments E8 to E12, further including lysing the red blood cells in a source of the cell population prior to culturing the source of the cell population.

E14. The method of any one of embodiments E8 to E12, further including selecting a fractionated source of the cell population by passage through a density gradient prior to culturing the source of the cell population.

E15. The method of any one of embodiments E8 to E14, wherein the cells of the cell population that co-express CD49c and CD90, do not express CD34 and/or CD45.

E16. The method of any one of embodiments E8 to E15, wherein the cells of the cell population that co-express CD49c and CD90 further express at least one cardiac-related transcription factor selected from the group consisting of GATA-4, Irx4, and Nkx2.5.

E17. The method of any one of embodiments £8 to E15, wherein the cells of the cell population that co-express CD49c and CD90 further express at least one trophic factor selected from the group consisting of:

### a) Brain-Derived Neurotrophic Factor (BDNF);

### b) Cystatin-C;

### c) Interleukin-6 (IL-6);

### d) Interleukin-7 (IL-7);

### e) Interleukin-11 (IL-11);

### f) Nerve Growth Factor (NGF);

### g) Neurotrophin-3 (NT-3);

### h) Macrophage Chemoattractant Protein-1 (MCP-1);

### i) Matrix Metalloproteinase-9 (MMP-9);

j) Stem Cell Factor (SCF); and,

### k) Vascular Endothelial Growth Factor (VEGF).

E18. The method of any one of embodiments E8 to E15, wherein the cells of the cell population that co-express CD49c and CD90 further express p21 or p53, and wherein expression of p53 is a relative expression of up to about 3000 transcripts of p53 per 10⁶ transcripts of an 18s rRNA and expression of p21 is a relative expression of up to about 20,000 transcripts of p21 per 10⁶ transcripts of an 18s rRNA.

E19. The method of any one of embodiments E8 to E15, wherein the isolated cell population has been cultured in vitro through a number of population doublings selected from the group consisting of

a) at least about 15 population doublings;
b) at least about 20 population doublings;
c) at least about 25 population doublings;
d) at least about 30 population doublings;
e) at least about 35 population doublings; and,
f) at least about 40 population doublings.

E20. Use of an isolated cell population according to any one of embodiments E1 to E7 in the manufacture of a medicament for treating a human suffering from a condition selected from the group consisting of:

a) a degenerative condition;
b) an acute injury condition;
c) a neurological condition; and,
d) a cardiac condition.

E21. Use of an isolated cell population according to any one of embodiments E1 to E7 in the manufacture of a medicament for treating a human suffering from a degenerative or acute injury condition.

E22. An isolated cell population derived from bone marrow, wherein greater than about 91% of the cells of the cell population co-express CD49c and CD90, and wherein the cell population has a doubling rate of less than about 30 hours under a low oxygen condition.

E23. The isolated cell population of embodiment E22, wherein the cell population is derived from human bone marrow.

E24. The isolated cell population of embodiments E22 or E23, wherein the low oxygen condition is between about 1 to 10% oxygen.

E25. The isolated cell population of embodiment E24, wherein the low oxygen condition is about 5% oxygen.

E26. The isolated cell population of any one of embodiments E22 to £25, wherein the cell population is cultured as an adherent cell population at a seeding density of less than about 2500 cells/cm².

E27. The isolated cell population of any one of embodiments E22 to E25, wherein the seeding density is less than about 1000 cells/cm².

E28. The isolated cell population of any one of embodiments E22 to E25, wherein the seeding density is less than about 100 cells/cm².]

E29. The isolated cell population of any one of embodiments E22 to E25, wherein the seeding density is less than about 50 cells/cm².

E30. The isolated cell population of any one of embodiments E22 to E25, wherein the seeding density is less than about 30 cells/cm².

E31. A method of making an isolated cell population, wherein greater than about 91% of the cells of the cell population co-express CD49c and CD90, and wherein the cell population has a doubling rate of less than about 30 hours, comprising the steps of:

a) aspirating bone marrow cells from a human;
b) lysing the red blood cell component of the bone marrow aspirate;
c) seeding the non-lysed bone marrow cells in a tissue culturing device;
d) allowing the non-lysed bone marrow cells to adhere to a surface;
e) culturing the adherent cells under a 5% oxygen condition; and
f) passaging the adherent cells at a seeding density of 30 cells/cm².

E32. An isolated cell population obtainable by the method of embodiment E31.

E33. An isolated cell population obtained by the method of embodiment E31.

E34. A method of making an isolated cell population, wherein greater than about 91% of the cells of the cell population co-express CD49c and CD90, and wherein the cell population has a doubling rate of less than about 30 hours after 30 cell doublings, comprising the steps of:
a) aspirating bone marrow cells from a human;
b) selecting a fractionated source of the cell population by passage through a density gradient;
c) seeding the fractionated cells in a tissue culturing device;
d) allowing the fractionated cells to adhere to a surface;
e) culturing the adherent cells under a 5% oxygen condition; and
f) passaging the adherent cells at a seeding density of 30 cells/cm².

E35. An isolated cell population obtainable by the method of embodiment E34.

E36. An isolated cell population obtained by the method of embodiment E34.

### EXAMPLES

### Example 1

### Bioactivity of Adult Bone Marrow-Derived Somatic Cells

### Production of Serum-Free Conditioned Media

Production of serum-free conditioned media was produced as described below for use in assays, such as the solid-phase antibody capture of secreted proteins (also as described below). Human exABM-SC (Lot # RECB-819; at ˜43 population doublings) were thawed and re-suspended in either Advanced DMEM (GIBCO™; Catalog #12491-015, Lot #1216032 (Invitrogen Corp., Carlsbad, Calif., USA)) supplemented with 4 mM L-glutamine (Catalog #SH30034.01. Lot #134-7944, (HYCLONE™ Laboratories Inc., Logan, Utah, USA)) or HyQ® RPMI-1640 (HYCLONE™ Catalog #SH30255.01, Lot # ARC25868) containing 4 mM L-glutamine and supplemented with Insulin-Transferrin-Selenium-A (ITS) (GIBCO™; Catalog #51300-044, Lot #1349264). Cell suspensions were then seeded in T-225 cm² CELLBIND™ (Corning Inc., NY, USA) culture flasks (culture surfaces treated with a patented microwave plasma process; see, U.S. Pat. No. 6,617,152) (n=3) at 20,000 cells/cm² in 36 mL of media (n=3 per condition). Cultures were placed in a 37° C. humidified trigas incubator (4% O₂, 5% CO₂, balanced with nitrogen) for approximately 24 hours. Cultures were then re-fed with fresh media on same day to remove non-adherent debris and returned to the incubator. On day 3, cell culture media were concentrated using 20 mL CENTRICON™ PLUS-20 Centrifugal Filter Units (Millipore Corp., Billerica, Mass., USA), as per manufacturer's instructions. Briefly, concentrators were centrifuged for 45 minutes at 1140×G. Concentrated supernatants were transferred to clean 2 mL cryovials and stored at −80° C. Fresh culture media were also concentrated as described for use as a negative control. The cells were then removed from the flasks using 0.25% porcine trypsin EDTA (CELLGRO™; Catalog #30-004-C1 (Mediatech Inc., Herndon, Va., USA)). Trypsin was then neutralized by adding back an equal volume of cell culture media containing 10% fetal bovine serum. Cell count and viability analysis was performed using a COULTER™ AcT 10 Series Analyzer (Beckman Coulter, Fullerton, Calif.) and trypan blue exclusion assays, respectively.

To perform 2D SDS-PAGE, human ABM-SC (Lot # PCH627; at 27 population doublings) were thawed and re-suspended in either HyQ® Minimum Essential Medium (MEM), Alpha Modification (HYCLONE™; Catalog #SH30265.01, Lot # ASA28110) supplemented with 4 mM L-glutamine (HYCLONE™; Catalog #SH30034.01, Lot #134-7944)) or RPMI1640 (HYCLONE™; Catalog #SH30255.01) supplemented with 4 mM L-glutamine (HYCLONE™; Catalog #SH30034.01, Lot #134-7944). Cell suspensions were then seeded in T-225 cm² CELLBIND™ culture flasks (11=3) at 24-40,000 cells/cm² in 36 mL of media (n=3 per condition). Cultures were placed in a 37° C. humidified trigas incubator (4% O₂, 5% CO₂, balanced with nitrogen) for approximately 24 hours. Cultures were re-fed with fresh media on same day to remove non-adherent debris and then returned to the incubator. The following day, conditioned media were collected, pooled, and centrifuged at 1140×G for 15 minutes to remove cell debris, and then transferred to sterile centrifuge tubes for short-term storage at −80° C.

### Example 2

### Two Dimensional (2-D) SDS PAGE Separation of Secreted Factors

### FIG. 1

Frozen aliquots of conditioned media and control media (samples) were shipped to Kendrick Labs, Inc. (Madison, Wis.) for analysis. Prior to use, samples were thawed and warmed to room temperature. Approximately 50 mL of each sample was lyophilized then re-dissolved in 200 microL of SDS Boiling Buffer (5% sodium dodecyl sulfate, 5% beta mercaptoethanol ethanol, 10% glycerol and 60 mM Tris, pH 6.8) and 2 mL of ultrapure water. The samples were then dialyzed against 5 mM Tris, pH 7.0 for two days at 4° C. using 6-8,000 MWCO membranes. The final dialysis was performed using water only. The samples were lyophilized once again, re-dissolved in 200 microL of SDS Boiling Buffer, and heated in a boiling water bath for 5 minutes before loading into the gels.

Two-dimensional gel electrophoresis was performed according to the method of O'Farrell (O'Farrell, P. H., J. Biol. Chem. 250: 4007-4021, 1975) as follows: Isoelectric focusing was first carried out in glass tubes of inner diameter 2.0 mm using 2.0% ampholines, pH 3.5-10 (Amersham Biosciences, Piscataway, N.J.) for 20,000 volt-hrs. 50 ng of IEF internal standard (tropomyosin) was then added to each sample. The tropomyosin standard is used as a reference point on the gel, it migrates as a doublet with a lower polypeptide spot of MW 33,000 and pI 5.2. The tube gel pH gradient for this set of ampholines was determined using a surface pH electrode.

After equilibration for 10 min in buffer 0 (10% glycerol, 50 mm dithiothreitol, 2.3% SDS, 0.0625 M tris, pH 6.8) each tube gel was sealed to the top of a stacking gel that, itself, is placed on top of a 12% acrylamide slab gel (1.0 mm thickness). SDS slab gel electrophoresis was carried out for about 5 hours at 25 mA. The following proteins (Sigma Chemical Co.) were added as molecular weight standards to a single well in the agarose portion of the gel (the agarose is cast between the tube gel to the slab gel): myosin (220,000 daltons), phosphorylase A (94,000 daltons), catalase (60,000 daltons), actin (43,000 daltons), carbonic anhydrase (29,000 daltons), and lysozyme (14,000 daltons). Following silver-staining the standards appear as bands on the basic edge of the acrylamide slab gel (Oakley et al. Anal. Biochem. 105:361-363, 1980). The gel was then dried between two sheets of cellophane paper with the acid end to the left (FIG. 1). If gels are intended for use with mass spectroscopy analysis they are stained using the silver stain method of O'Connell and Stults (O'Connell and Stults. Electrophoresis. 18:349-359, 1997).

The results show that using the methods provided, human ABM-SC can be cultured in the absence of animal serum to produce conditioned media rich in secreted proteins, and that such proteins can be individually identified and isolated. Conditioned media produced in such can also be processed, alternatively, by fractionating the expressed proteins based on a range of molecular weights. Techniques for protein concentration and fractionation are well-known and routinely used by those of ordinary skill in the art. These techniques include techniques such as affinity chromatography, hollow fiber filtration, 2D PAGE, and low-absorption ultrafiltration.

### Example 3A

### Pro-Regenerative Cytokine Secretion by Human ABM-SC

Human ABM-SC were plated in triplicate at 6,000 viable cells/cm² in cell culture “T” flasks containing AFG104 media. After allowing cells to attach and equilibrate for 24 hours, culture media was completely changed and flasks were incubated for 72 hours. Media was collected, centrifuged and stored at −80° C. until analysis for cytokines using commercially available colorimetric ELISA assay kits. For analysis of secreted cytokine release, sister flasks were treated with 10 mg/mL TNF-alpha, added during the last 24 hours of the 72 hour incubation. For each, lot three flasks of cells and supernatant were prepared, processed and banked independently for the basal and stimulated conditions, designated Basal Flask A, B and C or Stimulated Flask A, B and C, respectively.

Results show that when sub-cultured, ABM-SC secrete potentially therapeutic concentrations of several growth factors and cytokines known to augment angiogenesis, inflammation and wound healing. See, FIG. 11. Hence, ABM-SC have been shown to consistently secrete several cytokines and growth factors in vitro; including proangiogenic factors (e.g., SDF-1 alpha, VEGF, ENA-78 and angiogenin), immunomodulators (e.g., IL-6 and IL-8) and scar inhibitors/wound healing modulators (e.g., MMP-1, MMP-2, MMP-13 and Activin-A). Furthermore, the release of several of these factors is modulated by tumor necrosis factor alpha (TNF-alpha), a known inflammatory cytokine released during the course of acute tissue injury.

### Example 3B

### Solid-Phase Capture and Identification of Secreted Factors

### Table 1A, 1B and 1C

Conditioned media were screened for the presence of various proteins such as cytokines, proteases, and soluble receptors by solid phase antibody capture protein array, using RAYBIO™ Human Cytokine Antibody Array (RayBiotech, Inc., Norcross, Ga., USA). Briefly, frozen aliquots of conditioned media were thawed and warmed to room temperature prior to use. Array membranes were placed into the well of an eight-well tray (C series 1000). To each well, 2 mL 1× Blocking Buffer (RayBiotech, Inc.) was added and then incubated at room temperature for 30 min to block the membranes. Blocking Buffer was then decanted from each container, and the membranes were then incubated with conditioned media (diluted 1:10 with Blocking Buffer) at room temperature for 1 hr. Fresh cell culture media were used in place of PBS as negative controls. Samples were then decanted from each container and washed 3 times with 2 mL of 1× Wash Buffer I (RayBiotech, Inc.) at room temperature, while shaking for 5 min. Array membranes were then placed into one well, with 1 mL biotin-conjugated secondary antibody prepared in 1× Blocking Buffer, and incubated at room temperature for 1 hr. Arrays were then washed several times with Wash Buffer. 2 mL HRP-conjugated streptavidin diluted 1:1000 with 1× Blocking Buffer was added to each membrane and then incubated at room temperature for 2 hrs. Membranes were then washed several times with 1× Wash Buffer. Detection reagents for chemiluminescence were prepared as per manufacturer's instructions (RayBiotech, Inc.) and applied to each membrane and incubated at room temperature for 2 minute. Membranes were then placed protein side up on a plastic sheet. The opposite of the membrane was then covered with another piece of plastic sheet. Air bubbles were purged from the membranes by smoothing out the plastic. The membranes were then expose to x-ray film (Kodak X-OMAT AR™ film) and then processed using a film developer.

Table 1A, 1B and 1C shows an extensive list of cytokines, growth factors, soluble receptors, and matrix proteases secreted by human ABM-SC when sub-cultured in serum-free cell culture media. Media Supernatant Concentrate #1=Advanced DMEM (Gibco™) supplemented with 4 mM L-glutamine. Media Supernatant Concentrate #2=RPMI-1640 containing 4 mM L-glutamine and HEPES (HyClone) supplemented with Insulin-Transferrin-Selenium-A (Gibco™)

The results demonstrate that numerous trophic factors and soluble receptors important for tissue regeneration and modulation of the immune system are produced by ABM-SC when cultured under these conditions. Notably, earlier experiments demonstrated that supplementation of the base culture medium with insulin, transferrin, and selenium was required to achieve secreted protein levels such as those indicated in Table 1A, 1B and 1C. Protein levels shown in Table 1A, 113 and 1C were assessed using a RAYBIO™ Human Cytokine Antibody Array (RayBiotech, Inc.). Values are expressed as mean optical densities (O.D.). (N=2 for test samples. N=4 for controls.) Values reported with a (+) indicate mean O.D. values for that particular analyte greater than two standard deviations above the mean O.D. values for the respective negative control. Values reported with a (−) represent mean O.D. values for that particular analyte that are not greater than two standard deviations above the mean O.D. values for the respective negative control.

TABLE 1A

| | Media Supernatant | Media Supernatant |
|---|---|---|
| Cytokine | Concentrate #1 | Concentrate #2 |
| POSITIVE CTL | 11,020 (Mean O.D.) | 11,127 (Mean O.D.) |
| NEG CTL (Background) | 2,360.00 | 2,271.00 |
| Angiogenin | 5800.5 (+) | 4651 (+) |
| BDNF | 5855.5 (+) | 3587 (+) |
| BLC | 3852 (+) | 3164.5 (+) |
| BMP-4 | 3299 (+) | 2610 (+) |
| BMP-6 | 2359.5 (−) | 2290.5 (−) |
| CK beta 8-1 | 2408.5 (−) | 2426 (−) |
| CNTF | 2655.5 (+) | 2663 (+) |
| EGF | 3932.5 (+) | 2517 (+) |
| Eotaxin | 2527 (+) | 2488 (+) |
| Eotaxin-2 | 2467 (−) | 2452.5 (+) |
| Eotaxin-3 | 4564 (+) | 4450 (+) |
| FGF-6 | 2863.5 (+) | 2883.5 (+) |
| FGF-7 | 2328 (−) | 2374.5 (−) |
| Flt-3 Ligand | 2661 (+) | 2414.5 (−) |
| Fractalkine | 2432.5 (−) | 2379.5 (−) |
| GCP-2 | 2546.5 (+) | 2270 (−) |
| GDNF | 2299.5 (−) | 2208.5 (−) |
| GM-CSF | 2294 (−) | 2129 (−) |
| I-309 | 2431.5 (−) | 2222 (−) |
| IFN-gamma | 2807.5 (+) | 2848.5 (+) |
| IGFBP-1 | 3192 (+) | 4528.5 (+) |
| IGFBP-2 | 4813.5 (+) | 4244 (+) |
| IGFBP-4 | 4640 (+) | 4222.5 (+) |
| IGF-I | 2206.5 (−) | 2238 (−) |
| IL-10 | 2225.5 (−) | 2200.5 (−) |
| IL-13 | 2582 (+) | 2473 (+) |
| IL-15 | 2472.5 (−) | 2622.5 (+) |
| IL-16 | 2339.5 (−) | 2229.5 (−) |
| IL-1alpha | 2698.5 (+) | 2571.5 (+) |
| IL-1beta | 2276 (−) | 2253 (−) |
| IL-1ra | 2609 (+) | 2505.5 (+) |
| IL-2 | 2523.5 (+) | 2381 (−) |
| IL-3 | 2346 (−) | 2270 (−) |
| IL-4 | 2591 (+) | 2402 (+) |
| IL-5 | 3159 (+) | 3808 (+) |
| IL-6 | 45570 (+) | 40260.5 (+) |
| IL-7 | 7336.5 (+) | 5805 (+) |
| Leptin | 4187 (+) | 3733.5 (+) |
| LIGHT | 3689.5 (+) | 3378.5 (+) |
| MCP-1 | 9925.5 (+) | 5561 (+) |
| MCP-2 | 3117.5 (+) | 2481.5 (+) |
| MCP-3 | 2532 (+) | 2382 (−) |
| MCP-4 | 2702.5 (+) | 2694 (+) |
| M-CSF | 2387 (−) | 2381.5 (−) |
| MDC | 2414.5 (−) | 2510.5 (+) |
| MIG | 2344 (−) | 2342.5 (−) |
| MIP-1-delta | 2324 (−) | 2259.5 (−) |
| MIP-3-alpha | 2323.5 (−) | 2261.5 (−) |
| NAP-2 | 2517.5 (+) | 2467.5 (+) |
| NT-3 | 2973.5 (+) | 3205.5 (+) |
| PARC | 2668 (+) | 2630 (+) |
| PDGF-BB | 2580.5 (+) | 2780 (+) |
| RANTES | 2803 (+) | 2760 (+) |
| SCF | 2765 (+) | 2701.5 (+) |
| SDF-1 | 3721 (+) | 2562 (+) |
| TARC | 2488 (−) | 2395 (−) |
| TGF-beta 1 | 2381 (−) | 2311 (−) |
| TGF-beta 3 | 2422 (−) | 2531 (+) |
| TNF-alpha | 2243 (−) | 2321 (−) |
| TNF-beta | 2355 (−) | 2410.5 (−) |

TABLE 1B

| | Media Supernatant | Media Supernatant |
|---|---|---|
| Cytokine | Concentrate #1 | Concentrate #2 |
| POSITIVE CTL | 12,318 (Mean O.D.) | 11,936 (Mean O.D.) |
| NEG CTL | 2,452.00 | 2,392.00 |
| Acrp30 | 2539.5 (+) | 2436.5 (−) |
| AgRP | 2670 (+) | 2494 (−) |
| Angiopoietin-2 | 3372 (+) | 2656.5 (+) |
| Amphiregulin | 2692 (+) | 2447 (−) |
| axl | 3398.5 (+) | 3438.5 (+) |
| bFGF | 2915 (+) | 2901.5 (+) |
| Beta-NGF | 2573.5 (+) | 2544 (+) |
| BTC | 2653.5 (+) | 2554.5 (+) |
| CCL28 | 2706.5 (+) | 2553.5 (+) |
| CTACK | 3502 (+) | 3217 (+) |
| dtk | 2610.5 (+) | 2512 (+) |
| EGF-R | 3057.5 (+) | 2767.5 (+) |
| ENA-78 | 2630.5 (+) | 2503 (+) |
| Fas/TNFRSF6 | 3312 (+) | 3322.5 (+) |
| FGF-4 | 2711 (+) | 2650.5 (+) |
| FGF-9 | 2770 (+) | 2538.5 (+) |
| G-CSF | 3950.5 (+) | 3951 (+) |
| GITR ligand | 2973.5 (+) | 3107.5 (+) |
| GITR | 3198 (+) | 2935 (+) |
| GRO | 29446.5 (+) | 10214 (+) |
| GRO-alpha | 7351 (+) | 3553.5 (+) |
| HCC-4 | 3241 (+) | 2720.5 (+) |
| HGF | 5535 (+) | 3936.5 (+) |
| ICAM-1 | 3043 (+) | 2701.5 (+) |
| ICAM-3 | 2621.5 (+) | 2427 (−) |
| IGF-BP-3 | 3392 (+) | 3190.5 (+) |
| IGF-BP-6 | 5858 (+) | 6111 (+) |
| IGF-I SR | 2737.5 (+) | 2757 (+) |
| IL-1 R4/ST2 | 3463.5 (+) | 3235.5 (+) |
| IL-1 RI | 2522.5 (+) | 2401 (−) |
| IL11 | 2444.5 (−) | 2273 (−) |
| IL12-p40 | 2584 (+) | 2536 (+) |
| IL12-p70 | 2612 (+) | 2618 (+) |
| IL17 | 2610.5 (+) | 2555.5 (+) |
| IL-2 Ra | 2491 (−) | 2441.5 (−) |
| IL-6 R | 3202 (+) | 2836 (+) |
| IL8 | 24199.5 (+) | 17594.5 (+) |
| I-TAC | 3898 (+) | 3564 (+) |
| Lymphotactin | 3415.5 (+) | 3166 (+) |
| MIF | 3743 (+) | 3524 (+) |
| MIP-1-alpha | 2792 (+) | 2747.5 (+) |
| MIP-1-beta | 2638.5 (+) | 2523 (+) |
| MIP-3-beta | 2495.5 (−) | 2377 (+) |
| MSP-a | 2524.5 (+) | 2394 (−) |
| NT-4 | 2735 (+) | 2635 (+) |
| Osteoprotegerin | 4183.5 (+) | 3399 (+) |
| Oncostatin M | 2610 (+) | 2508 (−) |
| PlGF | 2705 (+) | 2493 (−) |
| sgp130 | 3232 (+) | 2866.5 (+) |
| sTNF RII | 3124 (+) | 3127 (+) |
| sTNF-RI | 9981 (+) | 7929.5 (+) |
| TECK | 2887.5 (+) | 2851 (+) |
| TIMP-1 | 8718 (+) | 9342.5 (+) |
| TIMP-2 | 11927 (+) | 12602 (+) |
| TPO | 3712 (+) | 3141.5 (+) |
| TRAIL-R3 | 3129 (+) | 3051 (+) |
| TRAIL-R4 | 3417 (+) | 3381 (+) |
| uPAR | 9557.5 (+) | 8158.5 (+) |
| VEGF | 8587.5 (+) | 6851 (+) |
| VEGF-D | 3477 (+) | 3190.5 (+) |

TABLE 1C

| | Media Supernatant | Media Supernatant |
|---|---|---|
| Cytokine | Concentrate #1 | Concentrate #2 |
| POS | 16,092 (Mean O.D.) | 15,396 (Mean O.D.) |
| NEG | 2,338 | 1,747 |
| Avtivin A | 23239.5 (+) | 18339 (+) |
| ALCAM | 14185.5 (+) | 15463.5 (+) |
| B7-1 (CD80) | 2983.5 (+) | 2222.5 (+) |
| BMP-5 | 2770.5 (+) | 2011.5 (+) |
| BMP-7 | 2564 (+) | 1828 (−) |
| Cardiotrophin-1 | 2816.5 (+) | 2097 (+) |
| CD14 | 3556 (+) | 2334.5 (+) |
| CXCL-16 | 4108.5 (+) | 2559 (+) |
| DR6 (TNFRSF21) | 3477 (+) | 2312 (+) |
| Endoglin | 3070 (+) | 2135 (+) |
| ErbB3 | 3366 (+) | 2313.5 (+) |
| E-Selectin | 2846.5 (+) | 1918 (+) |
| Fas-Ligand | 3531.5 (+) | 2943.5 (+) |
| ICAM-2 | 3158.5 (+) | 2155.5 (+) |
| IGF-II | 3212 (+) | 2395.5 (+) |
| IL-1 R II | 2855 (+) | 1834 (−) |
| IL-10 Rb | 2780 (+) | 1916 (+) |
| IL-13 Ra2 | 2559.5 (+) | 1693 (−) |
| IL-18 BPa | 2921 (+) | 1881 (−) |
| IL-18 Rb | 3238.5 (+) | 2387 (+) |
| IL-2 Ra | 3666 (+) | 2316.5 (+) |
| IL-2 Rb | 3001 (+) | 2083.5 (+) |
| IL-2 Rg | 3121 (+) | 2185.5 (+) |
| IL-21R | 3567.5 (+) | 2534.5 (+) |
| IL-5 Ra | 3084.5 (+) | 2237 (+) |
| IL-9 | 3676 (+) | 2324.5 (+) |
| IP-10 | 3300.5 (+) | 2262.5 (+) |
| LAP | 6202 (+) | 5383.5 (+) |
| Leptin R | 3487 (+) | 2791 (+) |
| LIF | 3486.5 (+) | 2400.5 (+) |
| L-Selectin | 3036.5 (+) | 2160 (+) |
| M-CSF R | 3140 (+) | 2330.5 (+) |
| MMP-1 | 3469 (+) | 2499 (+) |
| MMP-13 | 3083.5 (+) | 2316.5 (+) |
| MMP-9 | 3058.5 (+) | 2370 (+) |
| MPIF-1 | 2974 (+) | 2274.5 (+) |
| NGF R | 2887.5 (+) | 2355 (+) |
| PDGF-AA | 4130 (+) | 3423.5 (+) |
| PDGF-AB | 3191.5 (+) | 2278.5 (+) |
| PDGF Ra | 4430 (+) | 4027 (+) |
| PDGF Rb | 3768 (+) | 2784 (+) |
| PECAM-1 | 4071.5 (+) | 3450 (+) |
| Prolactin | 3199.5 (+) | 2151 (+) |
| SCF R | 3431.5 (+) | 2668.5 (+) |
| SDF-1b | 2268.5 (−) | 2156 (+) |
| Siglec-5 | 2691 (+) | 2160.5 (+) |
| TGF-a | 3058.5 (+) | 2388.5 (+) |
| TGF b2 | 3316 (+) | 2583 (+) |
| Tie-1 | 2883 (+) | 3178 (+) |
| Tie-2 | 3565 (+) | 3802.5 (+) |
| TIMP-4 | 6468 (+) | 6248 (+) |
| VE-Cadherin | 3164.5 (+) | 2428 (+) |
| VEGF R2 | 4030.5 (+) | 3003 (+) |
| VEGF R3 | 3200 (+) | 2651.5 (+) |

### Example 4

### Bioactivity of Adult Bone Marrow-Derived Somatic Cells

### In Vitro Neurogenesis Enhanced by Secreted Factors

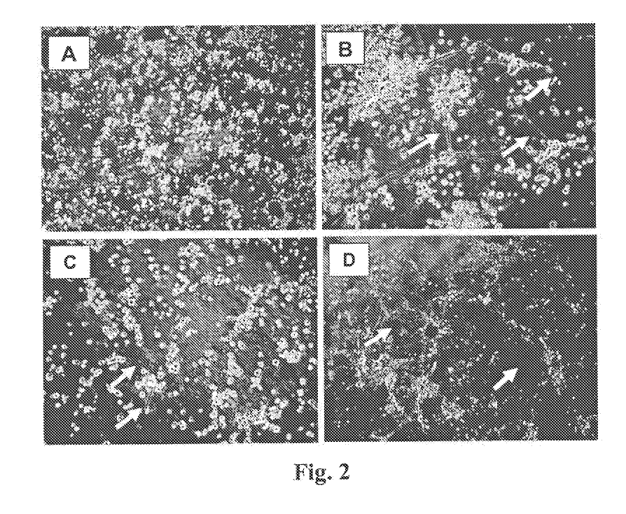

A stock solution of collagen was first prepared by re-suspending rat tail collagen (Sigma Chemical) in 0.1N acetic acid at a final concentration of 3.0 mg/mL. The collagen-based medium then was prepared as described by Bell et al., Proc. Natl. Acad. Sci. USA, vol. 76, no. 3, pp. 1274-1278 (March 1979) with minor modifications as described herein. Briefly, the collagen medium was prepared by mixing the rat tail collagen solution with DMEM 5× (JRH Biosciences) supplemented with 5 mM L-glutamine (CELLGRO™), Antibiotic-Antimycotic Solution (CELLGRO™), and a buffer solution (0.05N NaOH (Sigma Chemical), 2.2% NaHCO₃ (Sigma Chemical), and 60 mM HEPES (JRH Biosciences) at a ratio of 4.7:2.0:3:3. Approximately 500 microL of the collagen cell suspension was added to each well of a 24-well culture plate. The 24-well plates were then placed in a 37° C. humidified trigas incubator (4% O₂, 5% CO₂, balanced with nitrogen) for 1 hour to permit the collagen solution to congeal. Frozen rat PC-12 were thawed, washed in RPMI-1640 supplemented with 4 mM L-glutamine and HEPES (HYCLONE™) supplemented with Insulin-Transferrin-Selenium-A (GIBCO™) and centrifuged at 350×g for 5 minutes at 25° C. Cell pellets were re-suspended in same solution at a concentration of 75,000 viable cells/mL, with and without 136 ng/mL rat beta-NGF (β-NGF) (Sigma Chemical), 1:50 dilution of unconditioned concentrated RPMI-1640/ITS medium (used as a negative control), and a 1:50 dilution of conditioned concentrated RPMI-1640/Insulin-Transferrin-Selenium-A (ITS) media (media was conditioned as described in Example 1; conditioned and unconditioned, negative control media were concentrated as described in Example 1). Next, 1 mL of cell suspension was dispensed evenly across the surface of each of 2 gels (1 mL gel) for each cohort and then verified by phase contrast microscopy. The plates were then placed in a 37° C. humidified trigas incubator (4% O₂, 5% CO₂, balanced with nitrogen). Spent culture media was replaced every 3 days with fresh media. Images were captured on Day 10. See, FIG. 2.

These results demonstrate that PC12 differentiation into neurons by NGF is augmented dramatically when supplemented with conditioned media produced by human ABM-SC. Interestingly, the extent of neural differentiation, as assessed by the number of axon and neurites in the culture, was not significant when conditioned media was added alone. While some neurite outgrowth was observed in the presence of NGF alone, supplementing the cultures with conditioned media dramatically increased both the number and length of neurites. Previous work in our lab showed that supplementing RPMI culture media with insulin, transferrin, and selenium was critical for neural differentiation of PC12 under all standard published experimental conditions tested. These data indicate that media conditioned by human ABM-SC contain components which supplement or induce neurite outgrowth over and above the levels obtained with RPMI/ITS media alone or with RPMI/ITS media containing NGF. See, FIG. 2.

### Example 5

### Bioactivity of Adult Bone Marrow-Derived Somatic Cells

### Inhibition of Mitogen-Induced T Cell Proliferation In Vitro

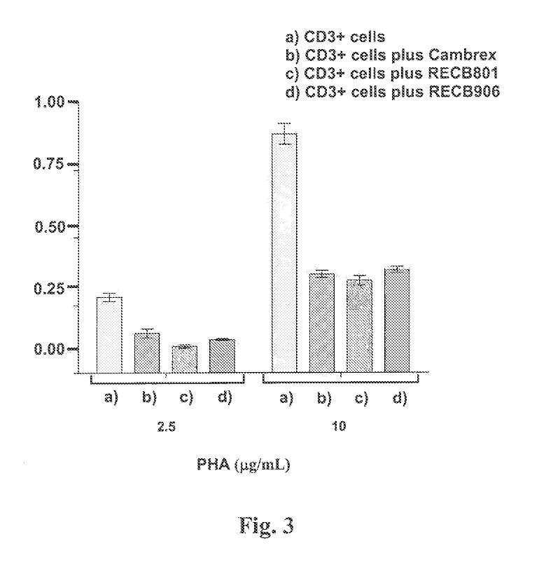

Human ABM-SC (Lot #RECB801 at ˜18 population doublings) and exABM-SC (RECB906 at ˜43 population doublings), were plated in 75 cm² flasks at a concentration of 6000 viable cells/cm² in complete media (Minimal Essential Medium-Alpha (HYCLONE™) supplemented with 4 mM glutamine and 10% sera-lot selected, gamma-irradiated, fetal bovine serum (HYCLONE™) and incubated at 37° C. in a humidified trigas incubator (4% O₂, 5% CO₂, balanced with nitrogen). After 24 hrs, spent media was aspirated and replaced with 15 mL fresh media. Human mesenchymal stem cells (hMSC, Catalog #PT2501, Lot #6F3837; obtained from Cambrex Research Bioproducts; now owned by Lonza Group Ltd., Basel, Switzerland) were plated in 75 cm² flasks at a concentration of 6000 viable cells/cm² in 15 mL Mesenchymal Stem Cell Growth Medium (MSCGM™; Lonza Group Ltd., Basel, Switzerland) and incubated at 37° C. in a humidified incubator at atmospheric O₂ and 5% CO₂. After 24 hrs, spent media was aspirated and replaced with 15 mL fresh MSCGM™. Both human ABM-SC (hABM-SC) and hMSC were harvested after 96 hours in culture. Harvested hABM-SC and hMSC were plated in 96-well round bottom plates at a concentration of 25,000 viable cells/mL in RPMI-complete media (HYCLONE™). Human peripheral blood mononuclear cells (PBMCs) were labeled in 1.25 microM CarboxyFluoroscein Succinimidyl Ester (CFSE) and cultured at 250,000 cells/well in RPMI-complete media along with hMSC, Lot #RECB801, Lot #RECB906 hABM-SC or alone. To stimulate T cell proliferation, cultures were inoculated with 2.5 or 10 microg/mL Phytohaemagglutinin (Sigma Chemical). Cells were then harvested 72 hrs later and stained with CD3-PC7 antibody (Beckman Coulter), as per manufacturer's instructions, and analyzed on a Beckman FC 500 Cytometer, using FlowJo 8.0 software (Tree Star, Inc., Ashland, Oreg.). Only CD3+ cells were analyzed for division index. See, FIG. 3.

These findings demonstrate that exABM-SC possess the capacity to inhibit T cell activation and proliferation and, therefore, may be useful as a therapeutic to suppress T cell-mediated graft rejection, autoimmune disorders involving dysregulation of T cells, or to induce a state of immune tolerance to an otherwise immunogenic skin product. Thus, one could envision the use of allogeneic human exABM-SC or compositions produced by such cells, to treat burn patients awaiting surgical application of an allogeneic skin product. In such an embodiment, treating an open wound first with exABM-SC, or compositions produced by such cells, may act not only to help rebuild the wound bed by inciting host cells to migrate to the cite of injury, but also to provide an environment permissive to long term engraftment of allogeneic skin or skin substitutes.

### Example 6

### Reconstitution of Porcine ABM-SC in Aqueous Vehicle for In Vivo Administration

Porcine ABM-SC were seeded at 60 cells/cm², refed at day 4, and grown for a total of 6 days. Cells were collected and frozen until subsequent use. Frozen aliquots of porcine ABM-SC were thawed, washed in DPBSG (Dulbecco's Phosphate Buffered Saline (CELLGRO™)) supplemented with 4.5% glucose) and centrifuged at 350×g for 5 minutes at 25° C. Cell pellets were re-suspended in DPBSG at a concentration of approximately 50,000/microL. Cell counts and viability assays were performed using a COULTER™ AcT 10 Series Analyzer (Beckman Coulter, Fullerton, Calif.) and by trypan blue exclusion, respectively. The cell suspension was then loaded into a 1 cc tuberculin syringe.

### Example 7

### Bioactivity of Adult Bone Marrow-Derived Somatic Cells

### Treatment of Incisional Wounds with Allogeneic Porcine ABM-SC

Two Yucatan swine, weighing between 57 kg and 78 kg were anesthetized and prepared for aseptic surgery. Four incisional wounds measuring approximately 50 mm in length were made with a scalpel blade on both sides of two animals (Nos. 3 and 4) for a total of eight wounds per animal along the paravertebral and thoracic area skin. Bleeding was stopped by inserting sterile gauze soaked with epinephrine into the lesion site. Gauze was then removed after about 10-20 minutes and each wound was treated with a single dose of porcine ABM-SC, divided into 12 separate injections evenly spaced around the incision with an additional 10-300 microL applied to the wound bed itself. Control wounds were injected similarly with vehicle only (DPBSG). Wounds were then closed with Steri-Strips™ (3M) and the animals were covered with protective aluminum jackets. The jackets were checked several times each day to ensure stable and proper position. The wound dressings were monitored daily and changes photographed on days 0, 1, 3, 5, and 7. Animals were euthanized on day 7 for histopathology. Formalin fixed paraffin embedded tissue sections were prepared and stained by H&E. Histomorphometric scoring was conducted by an expert veterinary pathologist blinded to the treatment group.

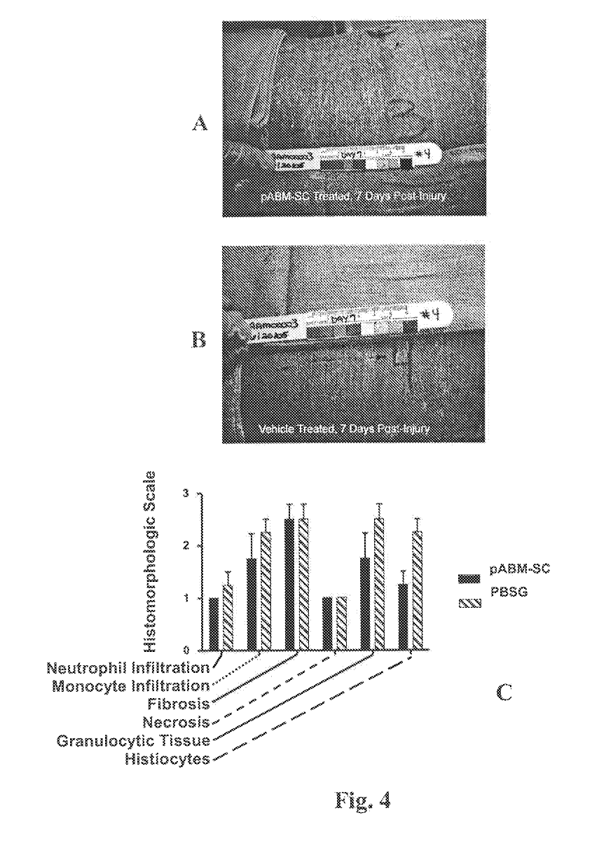

Seven days following treatment of the wounds, lesions treated with allogeneic porcine ABM-SC shown almost no signs of visible scarring (FIG. 4) while those treated with vehicle exhibited visible signs of scarring. Histomorphometric analysis of the wounds showed a marked reduction in tissue macrophages (histiocytes) in those treated with the ABM-SC, while no significant difference was seen in any of the other histological scores assessed.

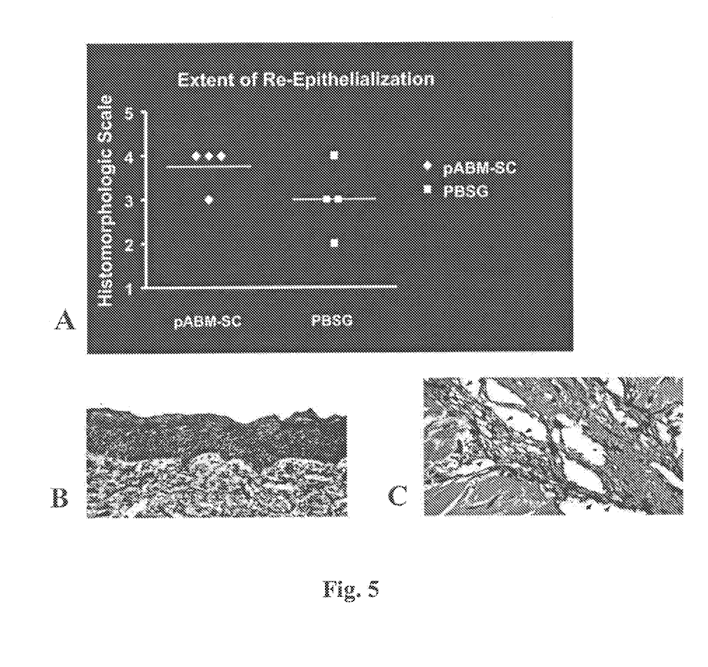

When similar tissue sections were scored for the extent of re-epithelialization (a crude indicator wound healing rate), those treated with ABM-SC exhibited a marked increase in the amount of epithelial cells repopulating the site of the incisions (FIG. 5).

### Example 8

### Bioactivity of Human ABM-SC in Collagen Vehicle for In Vivo Administration as a Liquid, Semi-Solid, or Solid-Like Therapeutic

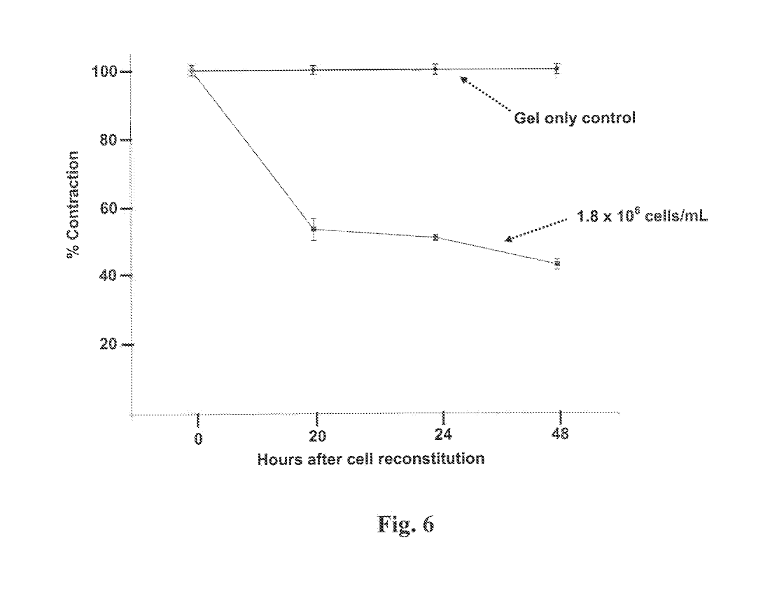

### FIG. 6-9

When reconstituted in a collagen-based biodegradable vehicle and stored at 4° C., human ABM-SC (Lot # PCH610; ˜27 population doublings) retain high cell viability for at least 24 hours (as demonstrated by cell bioactivity in gel contraction assays). Stored this way, the collagen solution will remain as a liquid and will preserve the cells in a suspended state without significant loss of viability (FIG. 6). Bioactivity of the cells can then be assessed using an in vivo assay of wound repair. To conduct this assay, a stock solution of collagen was first prepared by re-suspending rat tail collagen (Sigma Chemical) in 0.1N acetic acid at a final concentration of 3.0 mg/mL. The collagen medium was prepared as described by Bell et al. (Proc. Natl. Acad. Sci. USA, vol. 76, no. 3, pp. 1274-1278 (March 1979)) with minor modifications as described herein. Briefly, the collagen medium was prepared by mixing the rat tail collagen solution with DMEM 5× (JRH Biosciences) supplemented with 5 mM L-glutamine (CELLGRO™), Antibiotic-Antimycotic Solution (CELLGRO™; Catalog #30-004-C1), and a buffer solution (0.05N NaOH (Sigma Chemical), 2.2% NaHCO₃ (Sigma Chemical), and 60 mM HEPES (JRH Biosciences) at a ratio of 4.7:2.0:3.3. Frozen human adult bone marrow derived somatic cells (hABM-SC) were thawed, washed in DMEM 1× and centrifuged at 350×g for 5 minutes at 25° C. The cell pellets were re-suspended in DMEM I X at concentration of approximately 72,000 total cells/microL. Fifty microliters of cell suspension was then added to 2 mL collagen medium and gently triturated (i.e., gently pipetted up and down to obtain a homogeneous suspension of cells in collagen medium), yielding a final cell concentration of approximately 1,800 cells/microL. The cell suspension was then stored at approximately 4-8° C. overnight. The following day, the liquid cell suspension was transferred from the 15 mL conical tube and dispensed into 24-well cell culture plates at approximately 500 microL/well. The plates were then placed in a 37° C. humidified trigas incubator (4% O₂, 5% CO₂, balanced with nitrogen) for 1 hour to permit the collagen to solidify into a semi-solid gel. The gels were then removed from the 24-well plates using disposable sterile spatulas (VWR) and transferred to 12-well culture plates. The gels were then floated in 1.0 mL DMEM 1× per well. For negative controls, gels were prepared as described but without cells. Three wells were seeded for each condition (n=3).

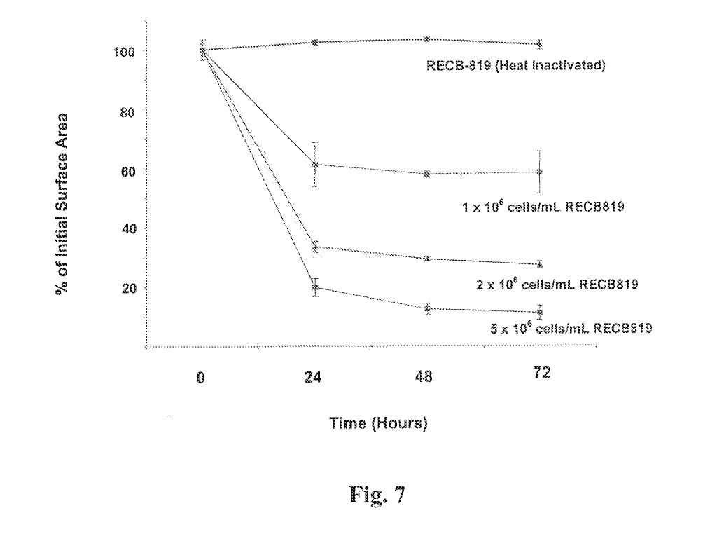

To evaluate the extent to which gel contraction is dose-dependent, a similar assay was conducted wherein human exABM-SC (Lot# RECB819; at ˜43 population doublings) were reconstituted in collagen solution at different cell concentrations immediately after removal from cryostorage (FIG. 7). A stock solution of collagen was first prepared by re-suspending rat tail collagen (Sigma Chemical) in 0.1N acetic acid at a final concentration of 3.0 mg/mL. The collagen medium then was prepared as described by Bell et al. (1979) with minor modifications as described herein. Briefly, the collagen medium was prepared by mixing the rat tail collagen solution with DMEM 5× (JRH Biosciences) supplemented with 5 mM L-glutamine (CELLGRO™), Antibiotic-Antimycotic Solution (Cellgro™), and a buffer solution (0.05N NaOH (Sigma Chemical), 2.2% NaHCO₃ (Sigma Chemical), and 60 mM HEPES (JRH Biosciences)) at a ratio of 4.7:2.0:3.3. Frozen human adult bone marrow derived somatic cells (hABM-SC) were thawed, washed in DMEM 1× and centrifuged at 350×g for 5 minutes at 25° C. The cell pellets were re-suspended in DMEM 1× at concentration of approximately 40,000, 80,000 and 200,000 viable cells/microL. Fifty microliters of each cell suspension was added to 2 mL collagen medium and gently triturated. Approximately 500 microL of the collagen cell suspension was added to each well of a 24-well culture plate. The plates were then placed in a humidified 37° C. trigas incubator (4% O₂, 5% CO₂ balanced with nitrogen) for 1 hour to permit the collagen solution to solidify. The gels were then removed from the plates using disposable sterile spatulas (VWR) and transferred to 12-well culture plates. The gels were floated in 1.0 mL DMEM 1× per well.

As a negative control, gels were prepared as described above using the highest concentration of hABM-SC (5×10⁶/mL) except that the cells were heat-inactivated (to eliminate biological activity). Heat-inactivated cells were first prepared by heating the initial cell suspension in DMEM 1× medium to 70° C. in a heat block containing water (heat transfer) for 40 minutes. Three wells were seeded for each condition (n=3).

To determine the extent to which the gels contracted over time, the percentage initial or starting surface area was calculated from digital images captured at 0, 24, 48 and 72 hours using a flatbed scanner. From each image, the diameter of the gel was measure both horizontally and vertically and then averaged. Results demonstrate that both the rate and extent of gel contraction was effected in a dose dependent manner (FIG. 7).

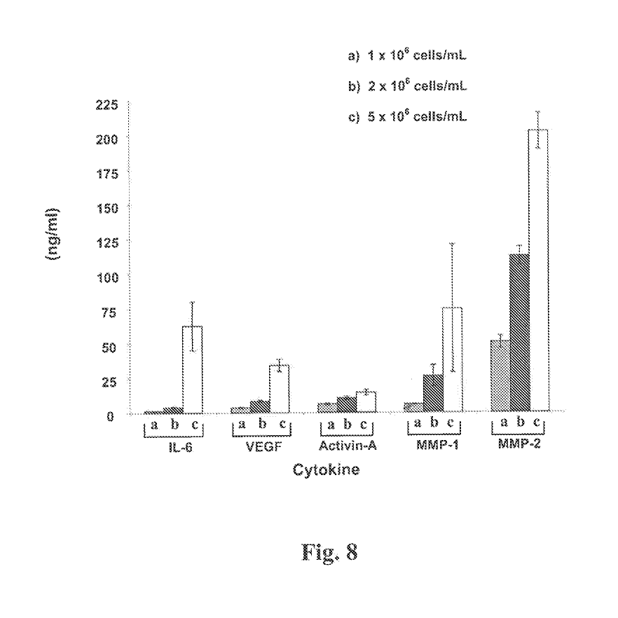

To determine the levels of certain secreted proteins produced from the human ABM-SC in these semi-solid gels, enzyme-linked immunosorbant assay (ELISA) was performed (on day 3 of culture) on conditioned cell culture supernatants collected from the liquid media surrounding the gels (FIG. 8). Supernatants were transferred to sterile 15 mL conical tubes and centrifuged at 1140×g for 15 minutes to remove cell debris. Supernatants were then transferred to 2 mL cryovials and transferred to −80° C. for short-term storage. On the day of assay, supernatants were thawed and equilibrated to room temperature before use. ELISA analysis was performed to detect IL-6, VEGF, Activin-A, pro-MMP-1, and MMP-2 ELISA (conducted as per manufacturer's instructions; all kits were purchased from R&D Systems, Inc. (Minneapolis, Minn., USA)). Results demonstrate that therapeutically relevant levels of trophic factors can be produced by these semi-solid neotissues and that these levels can be controlled by adjusting cell concentration. Of the trophic factors measured, detectable levels were not seen in cultures containing heat inactivated cells only. Statistical comparisons between assay conditions were determined by One-way ANOVA (*** p<0.001).

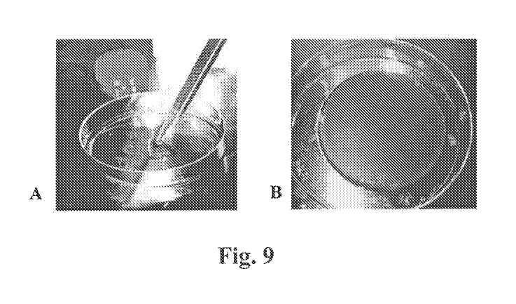

Human ABM-SC can also be reconstituted in a collagen solution to construct a large-format semi-solid structure that could be used as topical therapeutic (FIG. 9). To construct such a structure, a stock solution of collagen was first prepared by re-suspending rat tail collagen (Sigma Chemical) in 0.1N acetic acid at a final concentration of 3.0 mg/mL. The aqueous collagen medium was prepared by mixing the rat tail collagen solution with DMEM 5× (JRH Biosciences) supplemented with 5 mM L-glutamine (CELLGRO™, Antibiotic-Antimycotic Solution (CELLGRO™), and a buffer solution (0.286N NaOH (Sigma Chemical), 1.1% NaHCO₃. (Sigma Chemical), and 100 mM HEPES (JRH Biosciences) at a ratio of 6:2:2. Frozen hABM-SC were thawed and washed in 1×DMEM and then centrifuged at 350×g for 5 minutes at 25° C. The cell pellet was re-suspended in 1×DMEM at a concentration of approximately 90,000 cells/microL. Approximately 1.1 mL of cell suspension was then added to 20 mL collagen medium and gently triturated to achieve a final cell concentration of 5×10⁶ cells/mL. The final concentration of collagen was 1.8 mg/mL. The cell suspension was then dispensed into a 10 cm Petri dish (forming dish). The effective dose of cells in the collagen solution dispensed was approximately 100×10⁶ viable cells. The 10 cm forming dish containing the cell suspension was then placed in a humidified 37° C. incubator (5% CO₂) for 1 hour to permit the collagen solution to solidify. The semi-solid gel was then carefully removed from the 10 cm forming dish and transferred to a 15 cm Petri dish (culture dish) and photographed.

To construct a solid-like neotissue derived from human ABM-SC and collagen, the semi-solid structure described above can be placed back into a 37° C. humidified cell culture incubator (5% CO₂) for an additional 2 days (FIG. 10). To form a solid-like neotissue, a semi-solid gel prepared as described above, with the exception that the final collagen solution was 1.4 mg/mL (instead of 1.8 mg/mL), was carefully dislodged from the edges of the 10 cm forming dish and floated in approximately 82 mL 1×DMEM containing Antibiotic-Antimycotic Solution (CELLGRO™) in a 15 cm culture dish. The semi-solid gel was then transferred to a 37° C. humidified incubator (5% CO₂) for an additional 48 hrs to facilitate remodeling of the matrix into a solid-like tissue structure, free of the starting collagen substrate. The solid-like neotissue was then removed from the 15 cm culture dish and photographed (FIGS. 10A and 10B). Histological analysis of the neotissue by Masson's Trichrome stain demonstrates that the matrix is rich in newly synthesize human collagens and proteoglycans (FIG. 10C). Control collagen gels do not stain by this method. Collagens and proteoglycans stain blue.

The results of these studies indicate that frozen stocks of ABM-SC can be dispensed upon thaw and reconstituted in a liquid collagen-based medium that could be used therapeutically as a liquid suspension, semi-solid construct, or solid-like neotissue. When prepared in such a way and stored at approximately 4-10° C., the cell suspension will remain as liquid while maintaining satisfactory cell viability for greater than 24 hours. Employing such a method to formulate ABM-SC for clinical application then would provide considerable latitude to the clinician administering the cells. The suspension could be administered as a liquid injectable or, alternatively, could be applied topically to a wound bed. In the latter case, the liquid cell suspension would be anticipated to mold to the contour of the wound and then congeal into a semi-solid structure (for example, when warmed to 37 degrees C.). Alternatively, the suspension could be used in such a way as to manufacture semi-solid constructs or solid-like neotissues.

These data also show that when prepared by the methods, the resulting compositions each possess bioactivity important for mediating repair of various types of wounds, particularly those involving the skin.

ExCF-SC (for example, exABM-SC), or compositions produced by such cells, prepared in a liquid collagen-based medium could therefore be used topically to treat open wounds or as an injectable alternative to dermal fillers for facial rejuvenation.

In the semi-solid form, exCF-SC (for example, exABM-SC) or compositions produced by such cells, cold be used topically to treat severe burn patients that have had damaged full-thickness skin removed surgically, thereby acting as a dermal replacement.

Solid neotissues produced by exCF-SC (for example, exABM-SC) could be used surgically as an alternative to human cadaveric skin (ALLODERM™), porcine skin (PERMACOL™) and other animal-derived constructs (INTEGRA™). Moreover, these data also show that the potency of each of these various constructs can be controlled by altering dose of cells or compositions produced by the cells.

### Example 9

### Improvement of Cardiac Function in Rats Treated with hABM-SC

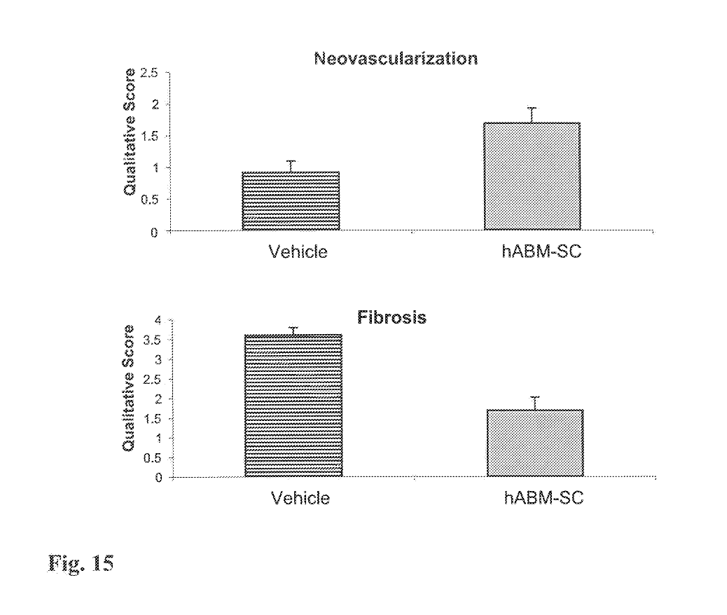

Administration of human ABM-SC to animals following myocardial infarct demonstrates that CF-SC (such as ABM-SC) improve cardiac function and enhance repair of cardiac tissue damage by stimulating angiogenesis and reducing fibrosis. See, FIG. 15. A rat model for acute myocardial infarction was utilized by occluding a coronary artery thereby creating a cardiac lesion (i.e., damaged region of heart). Lesioned rats were injected intercardially with either hABM-SC or vehicle.

Heart Function Methods: Sprague-Dawley rats of both sexes (age approx. 3 months) received experimentally-induced myocardial infarction via the placement of a permanent silk ligature around the left-anterior descending (LAD) coronary artery via a midline sternotomy. Five days after this procedure, the rats were begun on a standard regimen of Cyclosporine A treatment that lasted for the duration of the study. On day 7-8 following infarction, rats were anesthetized, intubated and an intercostal incision was made to expose the apex of the heart. An ultrasonic Millar catheter was then inserted through the ventricular wall, and pressure over time measurements were recorded for a period of approximately 30-60 seconds. This model of infarct production and pressure/nine measurements of cardiac function is a standard, well characterized model by which the effects of cellular therapies on cardiac function can be assessed (See e.g., Müller-Ehmsen, et al., Circulation., 105(14):1720-6 (2002)).

The test composition was delivered using a 100 microL Hamilton syringe fitted with a 30 gauge, low dead-space needle. Five separate injections of 20 microL were performed over the course of 2-3 minutes. Four injections were performed at equal distances around the visualized infarct, while the fifth was placed directly into the center of the infarcted region as determined by area of discoloration. After injection, the incision was sutured closed, the pneumothorax was reduced, and the animals were weaned from the respirator and extubated. Four weeks after injection (5 weeks post-infarction), animals were reanesthetized, the heart was exposed through a midline sternotomy, and a Millar catheter was inserted. Dp/dt measurements were taken as described above, after which the rats were euthanized via exsanguination.

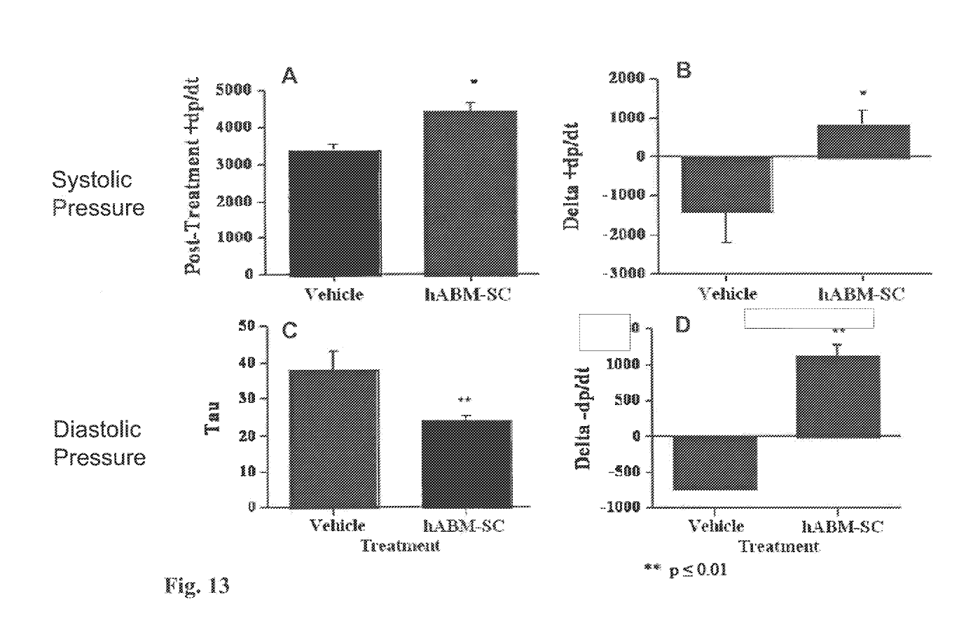

Heart Function Results (FIG. 13): Four weeks after treatment, rats receiving ABM-SC demonstrated significantly higher +dp/dt (peak positive rate of pressure change) values (A). Expressing changes in cardiac function over the course of the study by subtracting 0 week +dp/dt values from 4 week values (“delta +dp/dt”) demonstrated that while vehicle treated rats had decreases in cardiac function over the course of the study (negative delta), animals treated with either cell preparation showed significant improvement in cardiac function (B). Compared to vehicle treated rats, those receiving ABM-SC demonstrated significantly lower tau values (C), suggesting increased left ventricular compliance. Tau is the time constant of isovolumetric left ventricular pressure decay. For peak negative rate of pressure change (−dp/dt), expressing changes in cardiac function over the course of the study by subtracting 0 week −dp/dt values from 4 week values (“delta −dp/dt”) demonstrated that while vehicle-treated rats had decreases in cardiac function over the course of the study (negative delta), animals treated with cell preparation showed significant improvement in cardiac function (D). [*p<0.05, **p<0.01 by ANOVA]

Heart Structure Methods: Sprague-Dawley rats received experimentally-induced myocardial infarction via the placement of a permanent silk ligature around the left-anterior descending (LAD) coronary artery. Animals received a standard regimen of Cyclosporine A treatment (10 mg/kg s.c. daily) that lasted for the duration of the study.

On day 7-8 following infarction, rats were anesthetized, intubated and an intercostal incision was made to expose the apex of the heart. Cardiac function was accesses after which the test article was delivered using a 100 microL Hamilton syringe fitted with a 30 gauge, low dead-space needle. Five separate injections of 20 microL were performed over the course of 2-3 minutes. Four injections were performed at equal distances around the visualized infarct, while the fifth was placed directly into the center of the infracted region as determined by area of discoloration. After injection, the incision was sutured shut, the pneumothorax was reduced, and the animals were weaned from the respirator and extubated. Four weeks after injection (5 weeks post-infarction), animals were reanesthetized, the heart was exposed through a midline sternotomy, and cardiac function accessed. After functional measures were completed rats were euthanized via exsanguination. Rats were first deeply anesthetized using a mixture of ketamine (75 mg/kg) and medetomidine (0.5 mg/kg). The thoracic cavity was then surgically exposed and the heart dissected and immersion fixed in 10% neutral buffered formalin. Hearts were then grossly sectioned into three pieces, oriented into embedding molds, and processed for paraffin embedding. Heart tissues were then sectioned at 6 μm and stained by Hemotoxylin & Eosin (H&E) or Masson's Trichrome. At least six sections from every heart were also stained with hemotoxylin/eosin and Trichrome respectively. Specifically, trichrome staining allows for the visualization of collagen (blue) versus muscle tissue (red). Since collagen indicates the presence of scar tissue (absence of regeneration), the ratios of collagen to normal cardiac muscle were determined. A semiquantitative scoring scale was devised, with 0 as no detectable collagen and 5 as maximal/severe. Stained sections were then sent to a board certified pathologist for histomorphometric scoring.

Each slide contained three cross-sections of the heart, demonstrating a cross-sectional view of both ventricles from the mid-ventricular area (1) distal ⅓ of the ventricle (2), and apex of the ventricle (3). For histomorphometric analyses, the following grading scheme was used:

Location of tissue damage: Left ventricle (LV), Right ventricle (LV), Both ventricles (BV).

Percent of affected ventricle damaged (size of injury): Given in percent (0-100%)

Thickness score of experimentally damaged area of ventricle: Given a grade of 1-4 based on estimated thickness in millimeters. Compared with known landmarks in the tissue sections (e.g. average erythrocyte is 7 microns in diameter; average myocardial muscle bundle is 30 microns in diameter). Grade 1 (less than 0.5 mm); Grade 2 (0.5 mm to 1 mm); Grade 3 (1 mm to 1.5 mm); Grade 4 (1.5 mm+).

Neovascularization in area of tissue damage: (Grade of 0 to 4, from normal (0) to neovascularization throughout the entire area of initial tissue damage (4).

Initial vascular damage: Includes degeneration/necrosis of pre-existing blood vessels, with thrombosis and/or inflammation resulting from removal of remaining vascular debris, expressed as a grade of 0 to 4, with 0 being no vascular damage present, and 4 being vascular damage throughout the affected area.

Extent of fibrosis within the area of tissue damage: Expressed as a grade of 0 to 4, from no fibrosis (0) to (4) in 20% graduating levels of fibrosis and scarring of the initial area of damage caused by the infarction procedure. For example, fibrosis of 20% of the ventricle would be assigned a grade of (1), and fibrosis of 40% of the ventricle would be assigned a grade of (2), 60% would receive a (3), and above 60% would receive a (4).

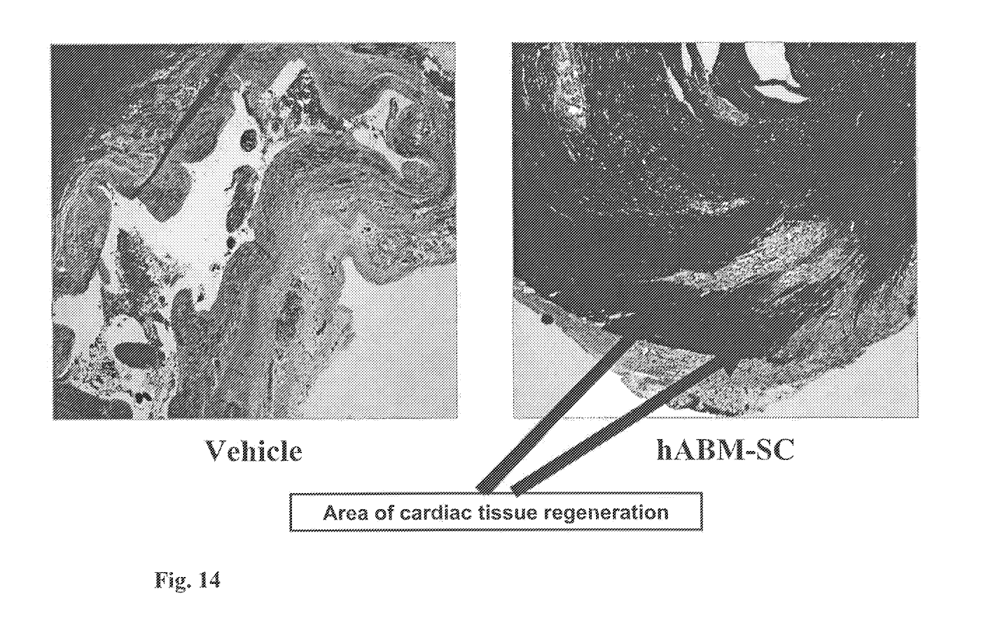

Heart Structure Results: Rats were subsequently sacrificed and cardiac tissue was sectioned and stained. A board certified veterinary pathologist performed semiquantitative scoring (FIG. 15) to evaluate changes in infarct size in the hearts of rats receiving vehicle or ABMSC seven days after myocardial infarction. Histopathological analysis indicated significant reduction in infarct size in rats receiving hABM-SC compared to vehicle. According to a preset scale, rats receiving hABM-SC had histological scores approximately two points lower than vehicle controls. FIG. 14 shows an example of typical infarct size reduction. Histopathological analysis determined that hABM-SC reduced fibrosis and increased vascularization in the infarct zone (FIG. 15), consistent with pro-regenerative activity. Thus, it was observed that rats treated with hABM-SC showed dramatic improvement of cardiac tissue structure. See, FIGS. 14 and 15.

### Example 10

### Adult Bone Marrow-Derived Somatic Cells Suppress Immune Mediated Responses

### Part I

### Suppression of Mitogen-Induced T-Cell Proliferation in One-Way MLR (Mixed Lymphocyte Reaction) Assay

Methods: Human ABM-SC and exABM-SC (Lot #RECB801 and RECB906, respectively), were plated in 75 cm² flasks at a concentration of 6000 viable cells/cm² in 15 mL complete media such as Advanced DMEM (GIBCO™; Catalog #12491-015, Lot #1216032 (Invitrogen Corp., Carlsbad, Calif., USA)) supplemented with 4 mM L-glutamine (Catalog #SH30034.01. Lot #134-7944, (HYCLONE™ Laboratories Inc., Logan, Utah, USA)) or HyQ® RPMI-1640 (HYCLONE™ Catalog #SH30255.01, Lot # ARC25868) containing 4 mM L-glutamine and supplemented with Insulin-Transferrin-Selenium-A (ITS) (GIBCO™; Catalog #51300-044, Lot #1349264) and incubated at 37° C. in a humidified trigas incubator (4% O₂, 5% CO₂, balanced with Nitrogen). After 24 hrs, spent media was aspirated and replaced with 15 mL fresh media. Human mesenchymal stem cells (hMSC) (Lonza BioScience, formerly Cambrex Bioscience, Catalog #PT2501, Lot 6F3837) were plated in 75 cm² flasks at a concentration of 6000 viable cells/cm² in 15 mL MSCGM™ (Lonza BioScience) and incubated at 37° C. in a humidified incubator at atmospheric O₂ and 5% CO₂. After 24 hrs, spent media was aspirated and replaced with 15 mL fresh MSCGM™. Both hABM-SC and hMSC were harvested after 96 hours in culture. Harvested hABM-SC and hMSC were plated in 96-well round bottom plates at a concentration of 25,000 viable cells/mL in RPMI-complete media (Hyclone). Human peripheral blood mononuclear cells (PBMCs) were labeled in 1.25 uM CarboxyFluoroscein Succinimidyl Ester (CFSE) and cultured at 250,000 cells/well in RPMI-complete media along with hMSC, RECB801, RECB906 or alone. To stimulate T cell proliferation, cultures were inoculated with 2.5 or 10 micrograms/mL Phytohaemagglutinin (Sigma Chemical). Cells were then harvested after 72 hrs later and stained with CD3-PC7 antibody (Beckman Coulter), as per manufacturer's instructions, and analyzed on a Beckman FC 500 Cytometer, using Flow Jo software. Only CD3+ cells were gated analyzed for division index.

Results: Allogeneic human ABM-SC and exABM-SC suppress mitogen-induced

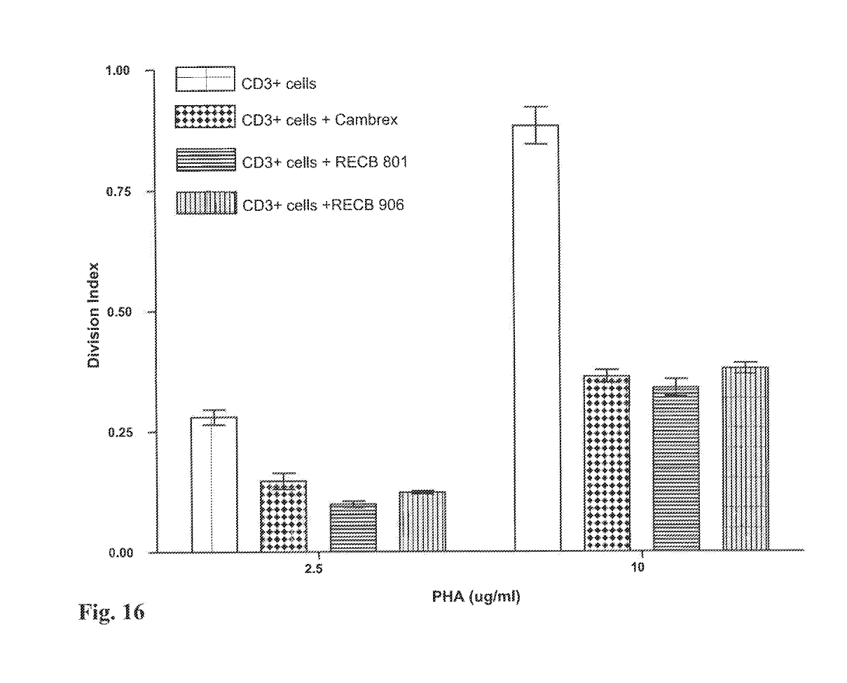

T-cell proliferation in one-way MLR assay. See, FIG. 16.

### Part II

### Allogeneic Porcine ABM-SC Fail to Illicit T-Cell Mediated Immune Response in 2-Way MLR Challenge Assay

Methods: Porcine whole blood was collected for immunoassays on Day 0 (prior to treatment) and at necropsy (Day 3 or Day 30 post-treatment) for cellular immune response analysis. PBMC from each animal were cultured with pABM-SC, the mitogen ConA, or media alone. Samples were analyzed by flow cytometry and the amount of proliferation calculated using FlowJo software.

Whole blood samples were diluted 1:1 with DPBS (Dulbecco's PBS)-2% Porcine Serum. Diluted blood was overlayed on Ficoll (2:1 ratio diluted blood to Ficoll) and centrifuged at 350×G for 30 minutes, with centrifugation cycle ending with zero braking. The resulting top layer was aspirated. The middle layers, which contain the desired mononuclear cells, were pooled for each sample, and washed in 3× with DPBS-2% Porcine Serum. After washing, the pellet was resuspended in 20 mL ACK lysis buffer and incubated for 3 minutes, to remove residual red blood cells, then centrifuged for 5 minutes, at 250×G. The pellets were washed in 20 mL DPBS-2% Porcine Serum and resuspended in 5 mL RPMI Complete media (RPMI-1640, 10% Porcine serum, 2 mM L-glut, 20 mM HEPES, 0.1 mM NEAA, 1× Penn-Strep). Cells were frozen at a concentration of 20×10⁶ cells/mL by centrifugation, and resuspension in ice cold freeze media (10% DMSO in Porcine Serum) and immediately added to 2 mL cryovials and placed into a cryorate freezer. (freeze rate=−25° C./min to −40° C., +15° C./min to −12.0° C., −1° C./min to −40° C., −10° C./min to −120° C.). Cells were stored in 2 mL aliquots per vial in vapor phase of liquid nitrogen until use.

On day 0, pABM-SC were plated in 96 well culture dishes at a concentration of 10,000 cells/well in AFG-104 media according to study template for each test condition. Plates were incubated overnight at 37° C. in a humidified incubator with low O₂ (4-5%), ˜5% CO₂ balanced with nitrogen.

The following day, PBMC were labeled with CFSE (carboxy-fluorescein diacetate, succinimidyl ester). In short, thawed vials of PBMC in 37° C. water bath, washed with 10 mL RPMI-Complete media centrifuged cells at 300×G, and resuspended in DPBS. Cell concentrations were adjusted to 10×10⁶ cells/mL and incubated with CFSE at a final concentration of 0.625 mM for 5 minutes. Cells were immediately washed in 40 mL ice cold DPBS/5% porcine serum and centrifuged 10 minutes at 300×G. Cells were again washed in 25 mL DPBS/5% porcine serum and centrifuged as before. Cells were washed a third and final time in 10 mL RPMI-complete media. Cell concentrations were adjusted to a final concentration of 5×10⁶ cells/mL. Labeled PBMC were added to the assay plate according to study template as follows: AFG-104 media was aspirated and replaced with 100 microL RPMI-Complete media. 100 microL of RPMI-complete media was added to non-stimulated wells, 100 microL media with 20 microg/mL ConA in RPMI-Complete media was added to stimulated wells, and 4.5% Glucose-RPMI-Complete media to vehicle cells. 500,000 labeled PBMC were plated per well in 96 well plates according to study template. Plates were incubated for 5 days at 37° C., atmospheric O₂ (high O₂), with humidity, 5% CO₂ no nitrogen. All conditions were completed in triplicate for each blood sample received. Vehicle stimulation was completed for a subset of blood samples, but was not significantly different than media alone. After 5 days of co-culture, cells were harvested for flow cytometry by transferring cells from 96 well plate to a flow tube. Indirect staining was conducted in accordance with standard protocols. The primary antibody used was Biotin Conjugated Mouse anti-Pig CD3 Monoclonal antibody; followed by exposure to Streptavidin-PE-Cy7 secondary reagent. Cells were resuspended in 200 microL flow wash buffer and analyzed on a Coulter FC500 device.

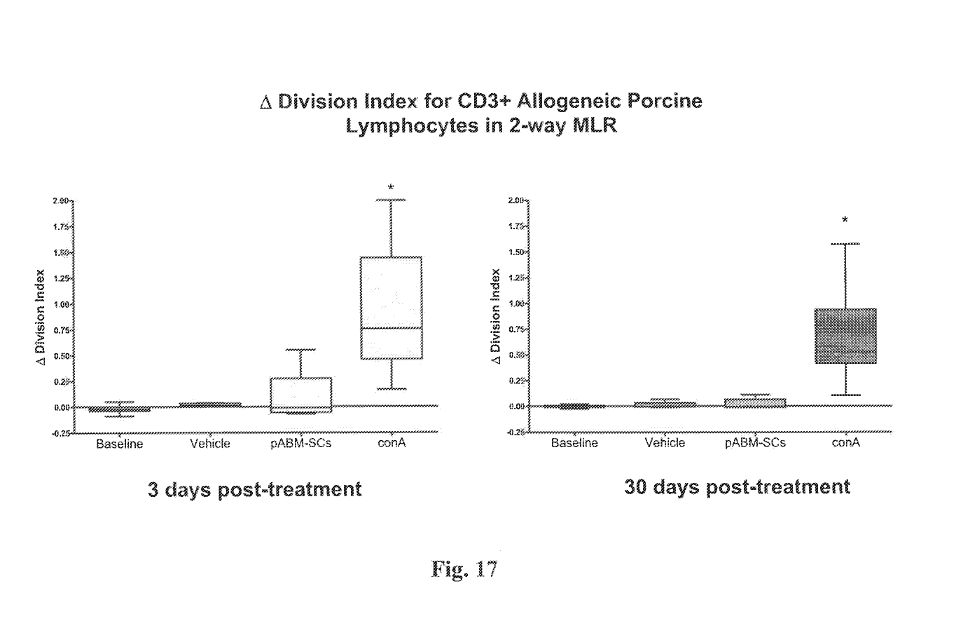

Results: A Division Index was calculated for, samples collected at baseline and at 3 or 30 days post-treatment and then challenged in vitro with media, vehicle, pABM-SC or ConA. The average division index from all animals at Day 3 or Day 30 for CD3+ cells stimulated with ConA was significantly higher than the division index for CD3+ cells from vehicle and pABM-SC treated animals at pre-treatment and at necropsy (*p<0.05). See, FIG. 17.

### Example 11

### Development

A Phase 1, open label, dose escalation study to evaluate the safety of a single escalating dose of hABM-SC administered by endomyocardial injection to cohorts of adults 30-60 days following initial acute myocardial infarction has been undertaken. The primary objective of this study was investigate the safety and feasibility of single escalating doses of hABM-SC delivered via multiple endomyocardial injections using the MYOSTAR™ catheter, guided by the NOGA™ or NOGA XP™ electromechanical cardiac mapping system. A secondary objective was to investigate the preliminary efficacy of single escalating doses of hABM-SC, measured by left ventricular volume, dimension, myocardial infarction size and voltage.

The study protocol provides that test subjects are to be followed for 12 months with frequent monitoring for safety. Efficacy assessments are to be performed at 90 day and six month follow-up visits. The intended study population is 30 to 75 year old consenting adults with an acute myocardial infarction (AMI) within the previous 30 days who have been successfully treated with percutaneous revascularization restoring TIMI II or higher flow, with a left ventricular ejection fraction of greater than or equal to 30% as measured by myocardial perfusion imaging (SPECT).

Inclusion and Exclusion Criteria: Inclusion criteria for the study comprises: (1) 30-75 years of age (inclusive); (2) 30-60 days since AMI (defined as the most recent MI causing a doubling in cardiac-specific troponin I (cTnI) enzyme concentrations relative to normal levels in addition to ECG changes consistent with MI with confirmation by myocardial perfusion imaging [SPECT]); (3) successful percutaneous revascularization of restoring TIMI II or higher flow to infarcted area; (4) negative pregnancy test (serum_hCG) in women of childbearing potential (within 24 hours prior to dosing); (5) left ventricular ejection fraction (LVEF)>30% as measured by myocardial perfusion imaging (SPECT); (6) cardiac enzyme tests (CPK, CPK MB, cTnI) within the normal range at baseline; (7) must be ambulatory.

Exclusion criteria for the study comprises: (1) significant coronary artery, stenosis that may require percutaneous or surgical revascularization within six months of enrollment; (2) left ventricular (LV) thrombus (mobile or mural); (3) high grade atrioventricular block (AVB); (4) frequent, recurrent, sustained (>30 seconds) or non-sustained ventricular tachycardia >48 hours after AMI; (5) clinically significant electrocardiographic abnormalities that may interfere with subject safety during the intracardiac mapping and injection procedure; (6) atrial fibrillation with uncontrolled heart rate; (7) severe valvular disease (e.g., aortic stenosis, mitral stenosis, severe valvular insufficiency requiring valve replacement); (8) history of heart valve replacement; (9) idiopathic cardiomyopathy; (10) severe peripheral vascular disease; (11) liver enzymes (Aspartate aminotransferase [AST]/alanine aminotransferase [ALT])>3 times upper limit of normal (ULN); (12) serum creatinine >2.0 mg/dL; (13) history of active cancer within the preceding three years (with exception of basal cell carcinoma); (14) previous bone marrow transplant; (15) known human immunodeficiency virus (HIV) infection; (16) evidence of concurrent infection or sepsis on chest X-ray (CXR) or blood culture; (17) participation in an experimental clinical trial within 30 days prior to enrollment; (18) alcohol or recreational drug abuse within six months prior to enrollment; (19) major surgical procedure or major trauma within the 14 days prior to enrollment; (20) known autoimmune disease (e.g., systemic lupus erythematosus [SLE], multiple sclerosis); (21) clinically significant elevations in prothrombin (PT) or partial thromboplastin time (PTT) relative to laboratory norms; (22) thrombocytopenia (platelet count <50,000/mm3); (23) inadequately controlled diabetes mellitus type I or type II, defined as a change in anti-diabetic medication regimen within the prior 3 months or HbA1c >7.0%; (24) uncontrolled hypertension defined as systolic blood pressure (SBP)>180 mmHg and/or diastolic blood pressure (DBP)>100 mmHg; (25) use of ionotrophic drugs >24 hours post AMI; (26) other co-morbid conditions such as hemodynamic instability, unstable arrythmias, and intubation, which, in the opinion of the principal investigator, may place subjects at undue risk or interfere with the objectives of the study; (27) any other major illness, which, in the opinion of the principal investigator, may interfere with the subject's ability to comply with the protocol, compromise subject safety, or interfere with the interpretation of the study results; and, (28) contraindication (either allergy or impaired renal function) to injection with contrast media for adequate CT scan evaluations.

Study Drug Dosage and Administration: Subjects in the same cohort will be dosed no closer than three days apart, and dosing of successive cohorts will be separated by approximately four weeks, following review of at least three weeks of safety data on all subjects in the previous cohort.

| Cohorts | Dose |
|---|---|
| Cohort 1 | 30 × 10⁶ cells |
| Cohort 2 | 100 × 10⁶ cells |
| Cohort 3 | 300 × 10⁶ cells |
| Cohort 4 | 300 × 10⁶ cells or MTD |

On the day of dosing, after baseline evaluations are complete and immediately following percutaneous ventricular mapping with the NOGA™ or NOGA XP™ electromechanical mapping system (Biosense Webster, Diamond Bar, Calif.), multiple sequential injections of hABM-SC will be delivered directly into the myocardium from a percutaneous, LV approach using a MYOSTAR™ catheter.

Study Procedures: Potential subjects will be consented and screened within 21 days prior to planned hABM-SC administration, which must occur within 30-60 days following AMI. Screening procedures to determine eligibility also will be used as baseline values, unless hospital SOPs require additional tests (i.e., immediately prior to catheterization). Baseline testing for treatment efficacy is to consist of a Six Minute Walk Test, NYHA classification, blood analysis for B-type natriuretic peptide (BNP) concentration, echocardiography, right and left cardiac catheterization, myocardial perfusion imaging (SPECT), and NOGA™ or NOGA XP™ electromechanical mapping. On the day of admission, additional baseline blood testing (including pregnancy testing [serum_hCG] for female subjects of childbearing potential) will be done, and eligibility will be verified. On the day of dosing (Study Day 0), subjects will undergo NOGA™ or NOGA XP™ electromechanical cardiac mapping and a MYOSTAR™ catheter will be placed into the left ventricle. The dose of hABM-SC will be administered via multiple sequential endomyocardial injections into the damaged (defined by NOGA™ or NOGA XP™) myocardial tissue. After administration of hABM-SC, echocardiography will be performed to detect possible transmural perforation, and the subject will be admitted directly to the intensive care unit (ICU) for a minimum of twenty-four hours of observation with continuous cardiac telemetric monitoring. Stable subjects without complications will be discharged from the ICU to a step down unit (with cardiac monitoring) and then discharged to home no sooner than 72 hours after the dosing procedure. Subjects with complications will remain in the ICU under optimal medical management until stable and appropriate for discharge to the step down unit. Safety evaluations will be performed 7, 14, 21, 60 and 90 days and at six and twelve months following administration of hABM-SC. Efficacy evaluations will be performed at 90 days and six months after the procedure, and will include left ventricular volume, dimension, size of myocardial infarction and voltage, measured respectively by contrast enhanced echocardiography, myocardial reperfusion and viability analysis (SPECT), right and left cardiac catheterization (90 days only), six minute walk test, NYHA classification, and NOGA™ or NOGA XP™ electromechanical mapping (90 days only).

Safety endpoints in the study will comprise: (1) adverse events as detailed in the study protocol; (2) clinically significant changes from baseline in blood or blood components including CBC, CMP, CPK/CPK MB, cTnl′, PT/PTT, and HLA antibody analysis; (3) clinically significant changes from baseline in cardiac electrical activity as assessed by electrocardiogram (ECG) or cardiac telemetry; (4) clinically significant changes from baseline in cardiac electrical activity as assessed by 24 hour Holter monitoring; (5) perioperative myocardial perforation as assessed by echocardiogram (post procedure); and, (6) clinically significant changes from baseline in physical and mental status as assessed by physical examination including a focused neurological examination. If signs and symptoms consistent with cerebrovascular accident (stroke) are observed, a neurological consult will be obtained for further evaluation

Efficacy Endpoints: Efficacy endpoints to be monitored comprise: (1) end systolic and/or end diastolic volume compared to baseline, as measured by myocardial perfusion imaging (SPECT); (2) myocardial infarction size compared to baseline as measured by myocardial perfusion imaging (SPECT); (3) end systolic and/or end diastolic dimensions compared to baseline as measured by contrast enhanced 2-D echocardiography; (4) action potential voltage amplitude in the area of hABM-SC injected myocardium as compared to baseline and historical controls (provided by the core laboratory) as measured by NOGA™ or NOGA XP™ electromechanical mapping; (5) cardiac output and pressure gradients compared to baseline as determined by right and left cardiac catheterization; (6) quality of life compared to baseline as assessed by the Six Minute Walk Test; and, (7) functional cardiovascular disease class (NYHA functional classification scheme) compared to baseline as assessed by the physician performing scheduled physical examinations.

Endomyocardial Delivery of hABM-SC: hABM-SC will be delivered to the myocardium via direct catheter-guided injection from within the ventricular chamber. Endomyocardial delivery of hABM-SC will be accomplished with the aid of the NOGA™ Cardiac Navigation System (one of the most advanced systems for three dimensional visualization of the physical, mechanical and electrical properties of intact myocardium in vivo; from Biosense-Webster, Diamond Bar, Calif.). The actual injection will be performed with the Cordis MYOSTAR™/catheter. The NOGA™ system allows for real time viewing of left ventricular heart function, detection of heart tissue damage, observation and placement of the catheter tip. Given the tenuous cardiac condition of patients post-AMI, a relatively non-invasive delivery system (compared to open heart or direct intracardiac delivery), i.e. the MYOSTAR™ Injection Catheter used in conjunction with the NOGA™ mapping system, was selected for administration of hABM-SC.

Preliminary Results: Preliminary results for 5 patients have been obtained. The first 3 patients comprised the initial dose group (30 million cells), while the last two patients received the second escalating dose (100 million cells). Overall, hABM-SC was well tolerated in all patients, with some trends to improvement in cardiac function noted in several patients. More detailed results are discussed below.

Safely Findings: No evidence of allogeneic immune response (as measured by pre- and post-treatment antibody profiling) was found in any patients.

Cardiac Functional Assessments: NOGA Electromechanical Mapping: Functional mapping was performed at time of treatment and at 90 days after cell treatment. Representative unipolar voltage maps were obtained from the second patient in the first dose cohort. A clear voltage deficit could be seen in the area of infarct (data not shown). Fifteen cell injections were performed at the margin of the infarct using unipolar voltage as a guide. At 90 days follow up, a clear improvement in unipolar voltage could be seen, with near normal voltages prevailing in the infarct zone. Similar degrees of improvement in voltage were noted in patients 1, 3 and 4 (data not shown).

Myocardial Perfusion Imaging (SPECT): Perfusion imaging was performed at baseline, 90 days and 6 months after cell treatment according to previously published methods.

All images were digitally captured and analyzed. Ejection fraction, perfusion deficit size, and ventricular volumes were derived from this analysis, under basal and adenosine-stress conditions, along with a 24 hour-washout rescan. Results for each patient at each time point are discussed below.

Perfusion Deficit: In general, perfusion deficit sizes, which are thought to represent overall infarct sizes, either decreased or remained unchanged over the six months of follow up for treated patients. Two patients demonstrated reductions in deficit deemed “clinically significant” meaning the deficits resolved to less than 4-5% of the total ventricular wall. In both of these cases, the areas of improvement corresponded to areas of voltage improvement as measured by NOGA mapping. Although NOGA mapping is considered investigational, this data supports validity of the hypothesis that unipolar voltage may be a surrogate for infarct size measurement.

Ejection Fraction (EF): In general, ejection fraction in study patients either improved or remained relatively unchanged. One patient experienced a significant drop in overall EF (63% to 50% over six months), but this patient experienced a serious adverse event during the course of cell treatment which renders it questionable whether or not a complete dose of cells was actually administered to the endomyocardium. Two patients demonstrated increases in EF well above the expected for this patient group. The lack of placebo controls precludes any conclusions as to the mechanism of this improvement.

End-Diastolic Volume (EDV): EDV was measure at baseline and at 90 days and 6 months following treatment. In general, EDV remained unchanged in all patients over the 6 month follow up period, suggesting no significant remodeling occurred in these patients.

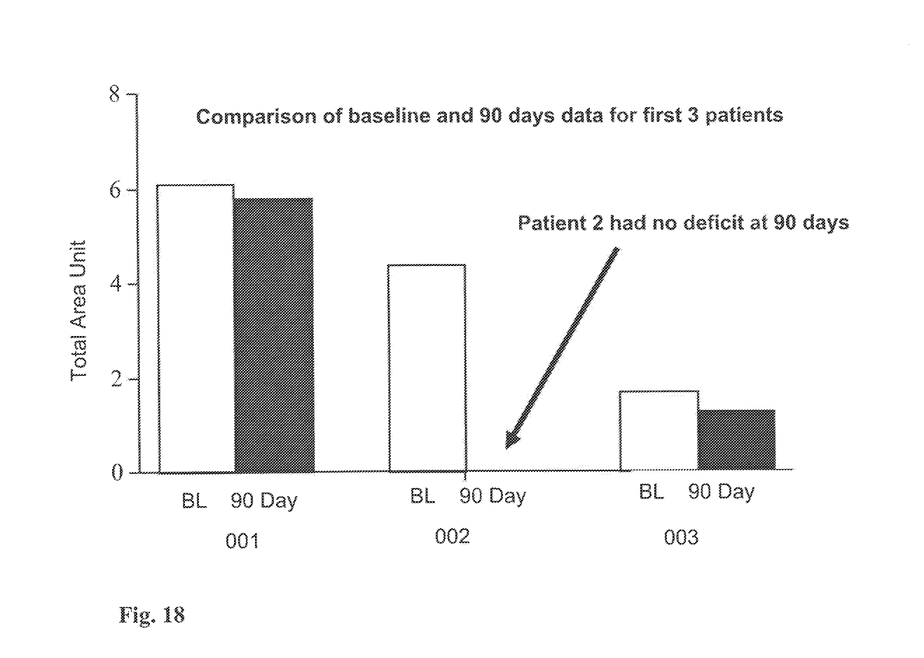

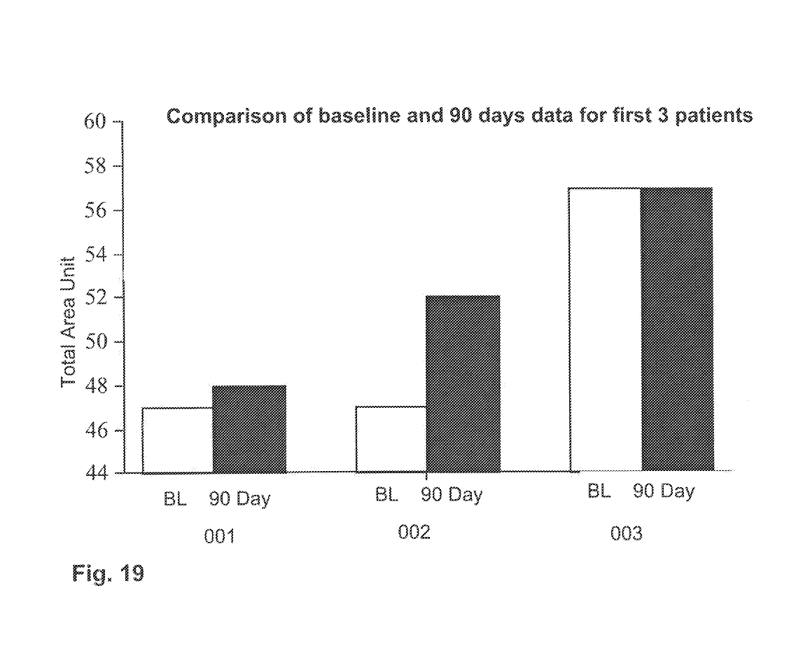

FIG. 18 shows the changes in cardiac fixed perfusion deficit size in three patients by comparison of a baseline (BL) measurements with measurements obtained 90 days post-treatment with hABM-SC. FIG. 19 shows the changes in cardiac ejection fractions measured in three patients by comparison of a baseline (BL) measurements with measurements obtained 90 days post-treatment with hABM-SC.

### Example 12

### Human ABM-SC and Compositions Derived Thereby for the Production of Red Blood Cells In Vitro

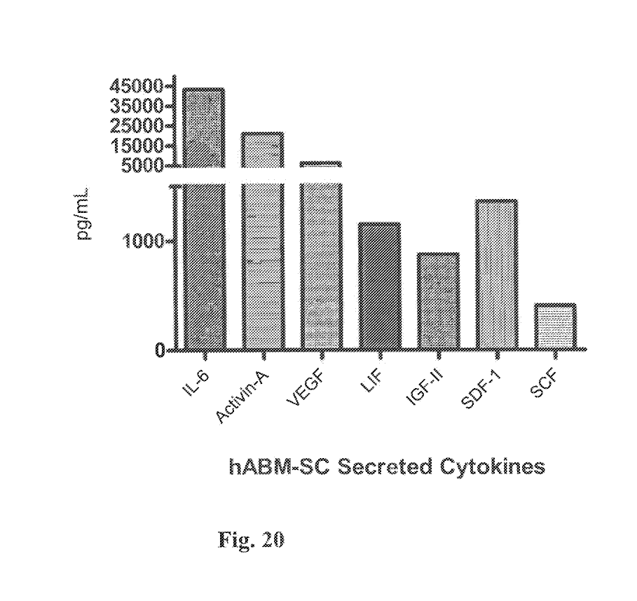

It is well known that the bone marrow microenvironment provides the requisite combination of matrix molecules, growth factors and cytokines necessary to support and modulate hematopoiesis (Dexter at al. 1981). Most, if not all, of the trophic factors known to drive hematopoietic cell self-renewal and lineage restricted differentiation derive from the mesenchymal support cells (Quesenberry et al. 1985). Roecklein and Torok-Storb (1995) showed that even within a relatively pure population of these cells, sub-populations can be isolated that differentially support hematopoiesis. Unlike the immortalized clones described in these previous publications, the hABM-SC utilized as described herein represent a pure population of CD45 negative, CD90/CD49c co-positive non-hematopoietic support cells that secrete many factors important for inducing and maintaining erythropoiesis including, but not limited to, IL-6 (Ullrich et al. 1989), LIF (Cory et al. 1991), SDF-1 (Hodohara et al. 2000), SCF (Dai et al. 1991), Activin-A (Shao et al. 1992), VEGF and IGF-II (Miharada et al. 2006) (FIG. 20).

To generate red blood cells from a starting population of hematopoietic precursors (e.g. embryonic stem cells (ES), hematopoietic stem cells (HSC), cord blood cells (CBC) or committed erythroblast precursors (BFU-E)), human ABM-SC and/or compositions produced by such cells can be utilized to induce, enhance, and/or maintain erythropoiesis by delivering a “cocktail” of erythropoietic factors necessary for, or to supplement, growth and differentiation of hematopoietic precursors into erythroblasts. See, FIG. 20.

### Example 13

### Production, Isolation, Purification, and Packaging of Cell-Derived Compositions and Trophic Factors

A two-step, downstream bioprocess has been developed to manufacture, collect and purify compositions such as secreted growth factors, cytokines, soluble receptors and other macromolecules produced by human ABM-SC and exABM-SC. This cocktail of secreted cell compositions, produced as such in the stoichiometric ratios created by the cells, has tremendous potential as a pro-regenerative therapeutic, cell culture reagent and/or research tool for studying in vitro cell and tissue regeneration. Such compositions can also be used as an alternative to the cells themselves to support the growth and lineage-appropriate differentiation of starting erythroid progenitor cell populations in suspension cultures.

### Production of Sera-Free Conditioned Media

Cryopreserved human ABM-SC (Lot no. P25-T2S1F1-5) are thawed and re-suspended in one liter of Advanced DMEM (GIBCO, catalog #12491-015, lot 284174 (Invitrogen Corp., Carlsbad, Calif., USA)) supplemented with 4 mM L-glutamine (HYCLONE Laboratories Inc., Logan, Utah, USA catalog #SH30255.01).

Cells are seeded in a Corning® CellBind® polystyrene CellSTACK® ten chamber (catalog number 3312, (Corning Inc., NY, USA)) at a density of 20,000 to 25,000 cells per cm². One port of the CellSTACK® ten chamber unit is fitted with a CellSTACK® Culture chamber filling accessory (Corning® Catalog number 3333, (Corning Inc., NY, USA)) while the other port is fitted with a CellSTACK® Culture chamber filling accessory 37 mm, 0.1 μM filter (Corning® Catalog number 3284, (Corning Inc., NY, USA)).

Cultures are placed in a 37° C.±1° C. incubator and aerated with a blood gas mixture (5±0.25% CO₂, 4±0.25% O₂, balance Nitrogen (GTS, Allentown, Pa.)) for 5±0.5 hrs. After 24±2 hrs post seeding, the media is removed, replaced with 1 liter of fresh media and aerated as previously described. Approximately, 72±2 hours later the sera-free conditioned media is aseptically removed from the CellSTACK® ten chamber unit within a biological safety cabinet and transferred to a one liter PETG bottle. The sera free conditioned media is subsequently processed by tangential flow filtration.

### Isolation and Purification of Sera-Free Conditioned Media

Tangential flow filtration (TFF) is performed on a reservoir of sera free conditioned media, recovered from a CellSTACK® ten chamber unit, as described above. A polysulfone hollow fiber with a molecular weight cut-off of 100 kilodaltons (kD) (Catalog number M1ABS-360-01P (Spectrum Laboratories, Inc., Rancho Dominguez, Calif., USA)) is employed. The reservoir of sera free conditioned (the retentate) is re-circulated through the lumen of the hollow fiber tangential to the face of the lumen. Molecules with a molecular weight of 100 kD or less pass through the lumen into a 2 liter PETG bottle; this fraction is called the permeate or filtrate. The retentate is continually re-circulated until the volume is reduced to approximately less than 50 mL. The retentate is subsequently discarded and the permeate is retained for further processing. The resulting permeate (approximately 1 liter) is a clear, sera-free solution containing small molecular weight molecules free of cellular debris and larger macromolecules, herein referred to as Fraction #1.

Fraction #1 is subsequently subjected to additional TFF using a polysulfone hollow fiber with a molecular weight cut off of 10 kilodaltons (kD) (Catalog number M11S-360-01P (Spectrum Laboratories, Inc., Rancho Dominguez, Calif., USA)). Fraction #1 is subsequently used as the retentate and re-circulated through the lumen of the hollow fiber, tangential to the face of the lumen. Smaller molecules ≦10 kD (i.e. ammonia, lactic acid etc.) are allowed to pass through the lumen. After the volume of the retentate is reduced to 100 mL, diafiltration of the solution is begun. One liter of alpha-MEM without phenol red (HYCLONE, catalog number RR11236.01 (HYCLONE Laboratories Inc., Logan, Utah, USA)) is added to the retentate reservoir at the same rate that the permeate is pumped out; thus maintaining the volume of the reservoir constant. The resulting retentate contains small only small molecules ranging in molecular weight from 10 kD to 100 kD; herein referred to as Fraction #2.

Fraction #2 can be further processed by subjecting it to additional TFF using a polysulfone hollow fiber with a molecular weight cut off of 50 kilodaltons (kD) (Catalog number MISS-360-01P (Spectrum Laboratories, Inc., Rancho Dominguez, Calif., USA)). Fraction #2 is thus re-circulated through the lumen of the hollow fiber, tangential to the face of the lumen. Smaller molecules ≦50 kD are passed through the lumen. Both processing streams are retained as product. The resulting permeate/filtrate is composed primarily of molecules 10 kD to 50 kD (Fraction #3), while the retentate comprises macromolecules in the range of 50 kD to 100 kD (Fraction #4).

Each of the resulting fractions is frozen in 60 mL PETG bottles (Catalog number 2019-0060, Nalgene Nunc International Rochester N.Y.).

Such Isolated protein fractions can subsequently be subjected to further aseptic downstream processing and packaging, wherein such compositions can be dialyzed, lyophilized, and reconstituted into a dry, biocompatible matrix, such as LYOSPHERES™ (manufactured by BIOLYPH™, Hopkins, Minn., USA).

### Example 14

### Isolation, Cryopreservation, and Expansion of CD34+Cord Blood Cells (CBC)

Large scale production of lineage-committed erythroid cells (CFU-E or Reticulocytes) can be manufactured from a starting population of stem cells or erythroblast precursors (e.g. cord blood cells, embryonic stem cells, hematopoietic stem cells and BFU-E) employing the methods described below.

Umbilical cord blood from healthy full-term newborns is collected in heparinized blood collection bags. A clean nucleated cell preparation is made by adding ammonium chloride lysis solution to cord blood, then centrifuging the mixture at 300×g for 15 minutes at room temperature. The supernatant is aspirated from the cell pellet, and the cell pellet is washed in BSSD with 5% human serum albumin (wash solution). The cells are centrifuged again at 300×g for 15 minutes at room temperature and the wash solution is removed from the cell pellet by aspiration. CD34+ cells are separated by magnetic cell sorting using MASC LS-columns (MACS®; Miltenyi Biotech, Gladbach, Germany) using established protocols. The CD34+CBCs are subsequently re-suspended in CSM-55 at approximately 2 million cells/mL and cryopreserved using a controlled-rate freezer.

BSSD (Balanced Salt Solution with 4.5% Dextrose) is prepared as follows:
To Balanced Salt Solution, Sterile Irrigating Solution (BSS; Baxter, Deerfield, Ill., USA) add 450±0.5 grams Dextrose (EMD Life Sciences, Gibbstown N.J. USA), QS to a final volume of 10.0 Liters with BSS.

CSM-55 (Cryogenic Storage Media 5% DMSO, 5% HSA) is prepared as follows:
In a 2 liter bottle combine 1.4 liters of BSSD with 400 mLs of 25% HSA (25% solution human serum albumin from ZLB Behring, Ill., USA) and 200 mLs of 50% DMSO (50% dimethyl sulfoxide from Edwards Lifesciences Irvine Calif. USA).

Wash solution is prepared with 400 mLs of BSSD plus 100 mLs of 25% HSA.
CD34+CBC Expansion in Suspension Cultures

The cells are subsequently re-suspended in StemSpan® H300 (StemCell Technology) supplemented with 1.0 U/mL recombinant human EPO(R&D Systems, Cat #287-TC), 10 LYOSPHERES™/L, and inoculated into a disposable HYCLONE™, perfusion BIOPROCESS CONTAINER™ (bioreactor) or equivalent, at a cell concentration of 1.0×10⁶/mL. Cultures are maintained at 37° C. with 5% CO₂, 4% O₂, and balanced with Nitrogen, for 3 weeks using continuous flow of fresh culture media. On day 14, cultures are supplemented with the glucocorticoid antagonist Mifepristone to accelerate enucleation, as described by Miharada et al. 2006. Continuous flow of fresh culture media is maintained at a fixed rate under these conditions until harvest on day 21.

### CBC Expansion on a Human ABM-SC Feeder Layer

Cryopreserved human ABM-SC are thawed and re-suspended in Advanced RPMI Media 1640 (INVITROGEN™) supplemented with 1.0 U/mL recombinant human EPO (R&D Systems, Cat 11287-TC), 4 mM L-Glutamine, 10% lot selected, gamma-irradiated fetal bovine serum (Hyclone), and seeded at a density of 10,000 cells/cm² in 40 layer cell culture factories (Corning) and maintained at 37° C. under 5% CO₂, 4% O₂, and balanced with Nitrogen at 37° C. On day 5, one-half volume of spent media is removed from the cultures and replenished by adding back one-half volume of fresh media along with 1.0×10⁶ CBC/mL. Discontinuous flow (on-off-on) of fresh culture media is subsequently engaged to enable the media conditions to cycle between fresh (on) to conditioned (off), and back to fresh media again (on). On day 14, cultures are supplemented with the glucocorticoid antagonist Mifepristone to accelerate enucleation, as described by above. Co-cultures are maintained under these conditions until harvest on day 21.

### Example 15

### ABM-SC Secrete Scavenger Receptors and Antagonists and Reduce Tumor Necrosis Factor-Alpha Levels in a Dose Dependent Manner

Background: Embodiments of the present invention include methods and compositions for treating, reducing, or preventing adverse immune activity (such as inflammation or autoimmune activity) in a subject by delivering therapeutically effective amounts of exABM-SC or compositions produced by exABM-SC. Embodiments of the invention include utilization of exABM-SC, or compositions produced thereby, relying on the naturally occurring or basal level production of secreted compositions in vitro. Alternatively, embodiments of the invention also include utilization of exABM-SC, or compositions produced thereby, by manipulating the exABM-SC to modulate (up- or down-regulate) the quantity and kind of compositions produced (for example, by administration of pro-inflammatory factors such as TNF-alpha).

For example, it has now been found that exABM-SC produce at least one scavenger receptor for the cytokine Tumor Necrosis Factor-alpha (TNF-α), and at least one antagonist of the Interleukin-1 Receptor (IL-1R), and at least one binding protein (antagonist) of cytokine Interleukin-18 (IL-18). Accordingly, embodiments of the invention include methods and compositions for use and administration of stable cell populations (such as exABM-SC) that consistently secrete therapeutically useful proteins in their native form.

The term “stable cell population” as used herein means an isolated, in vitro cultured, cell population that when introduced into a living mammalian organism (such as a mouse, rat, human, dog, cow, etc.) does not result in detectable production of cells which have differentiated into a new cell type or cell types (such as a neuron(s), cardiomyocyte(s), osteocyte(s), hepatocyte(s), etc.) and wherein the cells in the cell population continue to secrete, or maintain the ability to secrete or the ability to be induced to secrete, detectable levels of at least one therapeutically useful composition (such as soluble TNF-alpha receptor, IL-1R antagonists, IL-18 antagonists, compositions shown in Table 1A, 1B and 1C, etc.).

For purposes of the present invention, “scavenger receptor” is intended to mean any soluble or secreted receptor (whether membrane bound or free in the extracellular milieu) capable of binding to and neutralizing its cognate ligand.

In addition to the pro-inflammatory factors listed above, in view of the present disclosure it is also understood that cell populations of the present invention may be treated with any number, variety, combination, and/or varying concentrations of factors now known or subsequently discovered or identified in order to manipulate the concentration and kind of compositions produced by cell populations of the present invention. For example, the cell populations of the invention may preferably be treated with factors such as: IL-1 alpha, IL-1 beta, IL-2, IL-12, IL-15, IL-18, IL-23, TNF-alpha, TNF-beta, and Leptin. This brief list of preferred factors, however, is not intended nor should it be construed as limiting with respect to the number of different compositions that can be used to treat cell populations of the present invention, nor are these compositions limited to proteins, as is it is also appreciated that many other types of compounds could also be used to manipulate the cell populations of the present invention (including, by way of brief examples, other biological macromolecules such as nucleic acids, lipids, carbohydrates, etc. and small molecules and chemicals such as dimethylsulfoxide (DMSO) and nitrous oxide (NO), etc).

Methods: Production of serum-free conditioned media was produced as described below for use in enzyme-linked immunosorbant assays (ELISA) (also described below). Cryopreserved human exABM-SC (Lot # MFG-05-15; at ˜43 population doublings) were thawed and re-suspended in Advanced DMEM (GIBCO™; Catalog #12491-015, Lot #1216032 (Invitrogen Corp., Carlsbad, Calif. USA)) supplemented with 4 mM L-glutamine (Catalog #SH30034.01. Lot #134-7944, (HYCLONE© Laboratories Inc., Logan, Utah, USA)) with and without 10 ng/mL TNF-α. Cell suspensions were then seeded in T-225 cm² CELLBIND™ (Corning Inc., NY, USA) culture flasks (culture surfaces treated with a patented microwave plasma process; see, U.S. Pat. No. 6,617,152) (n=3) at 10,000, 20,000, 40,000 cells/cm² in 36 mL, of media (n=3 per condition). Heat-inactivated cells seeded at 40,000 cells/cm² were used as a negative control. Cells were heat-inactivated by transferring an aliquot to a sterile tube and incubating it for 40 minutes in a 70° C. heat block containing water (for efficient heat transfer). Cultures were placed in a 37° C. humidified trigas incubator (4% 0₂, 5% C0₂, balanced with nitrogen) for approximately 24 hours. Cultures were then re-fed with fresh media on same day to remove non-adherent debris and returned to the incubator. On day 3, cell culture media was concentrated using 20 mL CENTRICON™ PLUS-20 Centrifugal Filter Units (Millipore Corp., Billerica, Mass., USA), as per manufacturer's instructions. Briefly, concentrators were centrifuged for 45 minutes at 1140×G. Concentrated supernatants (100× final concentration) were transferred to clean 2 mL cryovials and stored at −80° C. until later use.

To determine the levels of certain secreted proteins produced from the human ABM-SC in these adherent cultures, enzyme-linked immunosorbant assays (ELISA) were performed on day 3, 100× concentrated, conditioned cell culture supernatants collected as described above. On the day of assay, supernatants were thawed and equilibrated to room temperature before use. ELISA analysis was performed to detect TNF-α, soluble TNF-RI (sTNF-RI), soluble TNF-RII (sTNF-RII), IL-1 receptor antagonist (IL-IRA) and IL-2 receptor alpha (conducted as per manufacturer's instructions; all kits were purchased from R&D Systems, Inc. (Minneapolis, Minn., USA)).

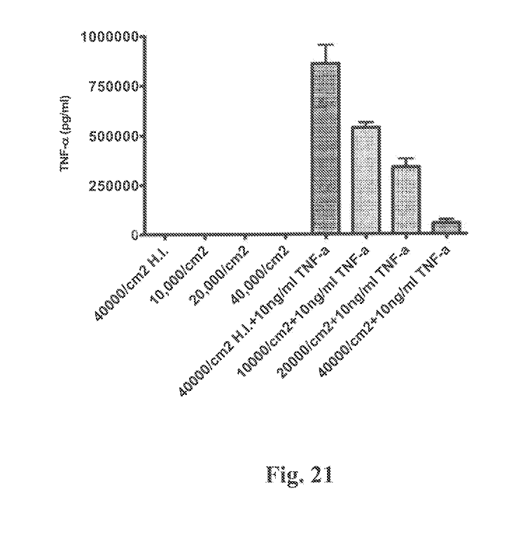

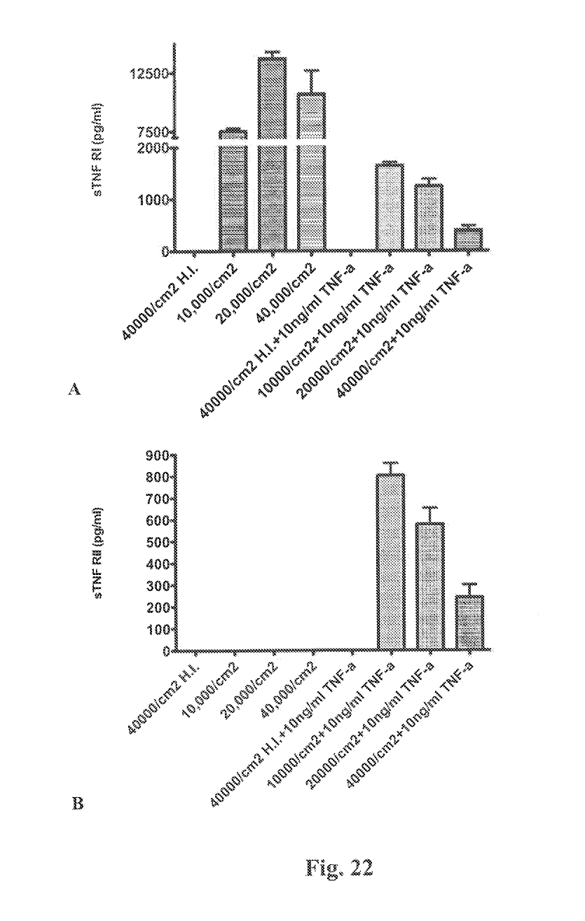

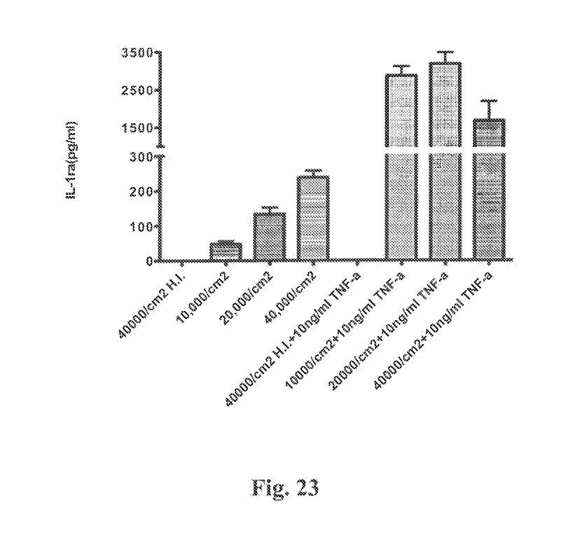

The results demonstrate that therapeutically relevant levels of secreted scavenger receptors (e.g. sTNF-RI) and receptor antagonists (e.g. IL-IRA) are produced by these adherent cultures and that these levels can be controlled by adjusting cell concentration or dose (FIG. 21-23). Importantly, these data also demonstrate that the cells respond to the inflammatory milieu in which they are placed. For example, following treatment with the potent inflammatory cytokine TNF-alpha, the cells up-regulate secretion of sTNF-RII (FIG. 22B) and IL-IRA (FIG. 23). Interestingly, in these sample cultures, the levels of TNF-alpha were significantly reduced with each increase in cell seeding density (FIG. 21), suggesting that the TNF-alpha itself was sequestered in some way by either the ABM-SCs or factors that they secrete.

It is well established that both sTNF-RI and sTNF-RII can bind and neutralize the biological activity of TNF-alpha. Since the measurable levels of both forms of the TNF receptor, as well as TNF-alpha itself, are each reduced significantly with each increase in cell seeding density, it is likely that the ABM-SC derived sTNF-RI and sTNF-RII are binding to and masking TNF-alpha in this assay system.

Of the soluble receptors and receptor antagonists measured, detectable levels were not seen in cultures containing heat-inactivated cells only. Statistical comparisons between assay conditions were determined by one-way ANOVA.

### Example 16

### Osteogenesis Induction Assay

### Human ABM-SC Cells do not Exhibit a Bone Differentiation Characteristic In Vitro when Cell Populations Expanded Beyond Approximately 25 Population Doublings are Exposed to Standard Osteoinductive Conditions or when Cell Populations Expanded Beyond Approximately 30 Population Doublings are Exposed to Enhanced Osteoinductive Conditions

Methods: Human ABM-SC and exABM-SC were seeded at 3100 cells/cm² in 6-well culture dishes (Corning, Catalog #3516) with 2.4 mL Mesenchymal Stem Cell Basal Medium (MSCBM™; Lonza, Catalog #PT-3238) supplemented with MSCGM™ SingleQuot Kit (Lonza, Catalog #PT-4105) per well, hereafter referred to as Mesenchymal Stem Cell Growth Medium (MSCGM™). Approximately four hours later, the MSCGM™ was changed to the appropriate test conditions. Negative control wells were those re-fed with either MSCGM™ alone, or MSCGM™ supplemented with 5 ng/mL recombinant mouse Noggin/Fc Chimer (R&D Systems, Catalog #719-NG). The test wells were those treated with either Osteogenesis Induction Medium (OIM; Lonza Catalog #PT-3924 and #PT-4120) alone (standard osteoinductive conditions) or OIM supplemented with 5 ng/mL recombinant mouse Noggin/Fc Chimer (enhanced osteoinductive conditions). Cultures were then maintained in a humidified CO₂ incubator at 37° C. and re-fed with fresh medium every 3-4 days for 2 weeks. After 14 days, cultures were processed for calcium determination using the Calcium Liquicolor kit (Stanbio, Catalog #0150-250), as per manufacturer's instructions. Plates were read at 550 nm using a SpectraMax Plus³⁸⁴ microplate reader.

Results: Human ABM-SC and exABM-SC derived from research lot # MCB109 were cultured under standard osteoinductive conditions (OIM only) or under enhanced osteoinductive conditions (OIM and the morphogen Noggin; OIM+Noggin). Negative control cultures were maintained in either growth media alone (MSCGM™) or MSCGM™ supplemented with Noggin (MSCGM™+Noggin).

ABM-SC at about 16 population doublings exhibited a calcium deposition increase of approximately 6-fold when the OIM media was supplemented with Noggin (i.e., ABM-SC at about 16 population doublings deposited ˜5 micrograms calcium/well under OIM conditions and ˜30 micrograms/well under OIM+Noggin conditions). ABM-SC lost the capacity to deposit detectable levels of calcium beyond about 16 population doublings under standard OIM conditions, however, this could be reversed by supplementing with Noggin (i.e., exABM-SC at about 25 population doublings deposited no detectable calcium under OIM conditions whereas these same cells deposited ˜5 micrograms calcium/well under OIM+Noggin conditions). In contrast, beyond about ˜30 population doublings (e.g., at about 35 and 43 populations doublings) exABM-SC did not deposit detectable levels of calcium under any of the conditions tested (standard or enhanced OIM).

### Example 17

### Expression of IL-1 Receptor Antagonist (IL-IRA) and IL-18 Binding Protein (IL-18BP) by ABM-SC

Methods: Human ABM-SC which had undergone about 43 cell population doublings (lot # P17-T2S1F1-5) were thawed and seeded in AFG growth medium supplemented with Brefeldin A at 3 micrograms/mL (1×) and placed in a humidified 5% CO₂ incubator at 37° C. for 24 hours. Cultured cells were then removed from the culture flasks using porcine trypsin, washed and prepared for flow cytometry, as per CALTAG FIX & PERM® staining protocol (CALTAG LABORATORIES; now part of Invitrogen Corp. (Carlsbad, Calif., USA). Cells were stained with either FITC conjugated mouse anti-human IL-1 Receptor Antagonist (IL-IRA; eBioscience, Catalog #11-7015, clone CRM17) antibody neat or unlabeled rabbit anti-IL-18 Binding Protein (IL-18BP; Epitomics, Catalog #1893-1, clone EP1088Y) at a 1:10 dilution, both for 45 minutes at room temperature. FITC-rabbit FITC-labeled goat anti-rabbit antibody was then used to detect the IL-18BP. Isotype matched controls were included as a negative control (Beckman Coulter).

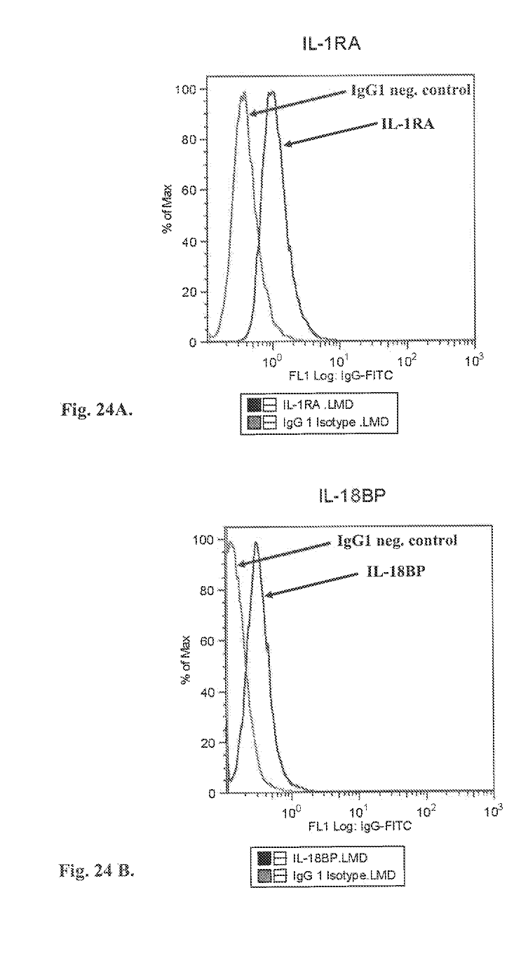

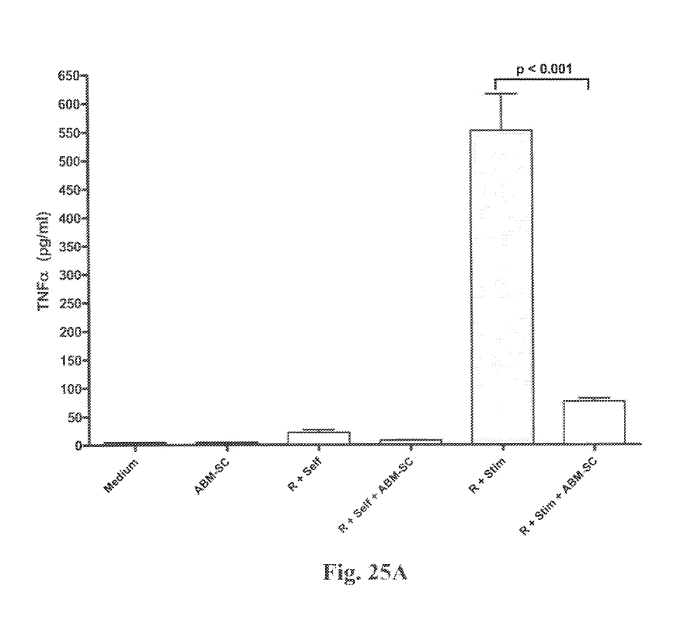

Results: Human exABM-SC express basal levels of IL-1 receptor antagonist (IL-IRA; FIG. 24A) and IL-18 binding protein (IL-18BP; FIG. 24B) even in the absence of an inflammatory signal such as TNF-alpha.

### Example 18

### Human ABM-SC Reduce Expression of TNF-Alpha and IL-13 while Simultaneously Increasing Expression of IL-2

Methods: Human peripheral blood mononuclear cells (PBMC) were co-cultured in RPMI-1640 containing 5% Human Sera Albumin, 10 mM HEPES, 2 mM glutamine, 0.05 mM 2-mercaptoethanol, 100 U/mL penicillin, and 100 microg/mL streptomycin, in a 24 well plate with either 1) Mitomycin-C treated PBMC from same donor (Responder+Self) or 2) Mitomycin-C treated PBMC derived from a different donor (Responder+Stimulator). PBMC from each source were each seeded at 4×10⁵ cells/well. For each condition, cultures were supplemented with or without human ABM-SC at a seeding density of 40,000 cells/well. Cultures were maintained in a humidified 5% CO₂ incubator at 37° C. for 7 days to condition the media. Conditioned cell culture supernatants were collected and analyzed for the presence of the various cytokines using the SEARCHLIGHT™ 9-Plex assay (Pierce Protein Research Products, Thermo Fisher Scientific Inc., Rockford, Ill.). Statistical analysis was performed by one-way ANOVA (analysis of variance).

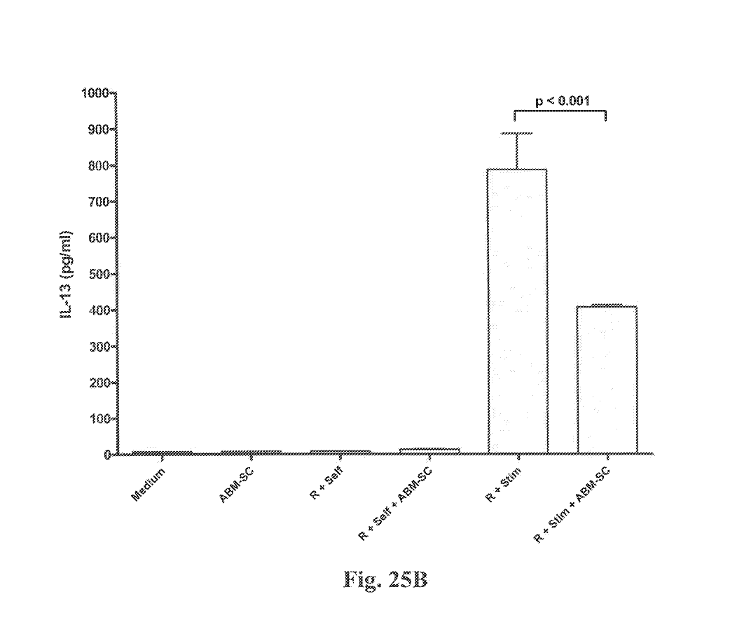

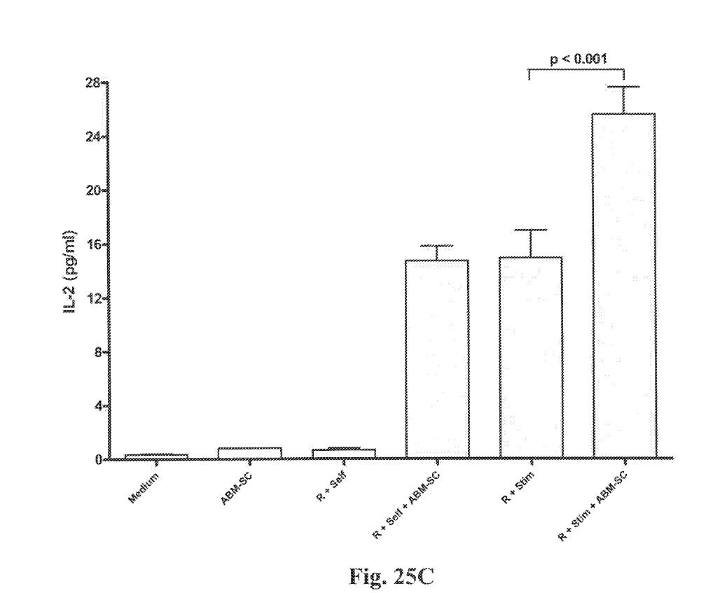

Results: Co-culture of allogeneic PBMC (Responders+Stimulators) resulted in a marked increase in the levels of TNF-alpha and IL-13, as would be expected for a mixed PBMC reaction. When challenged with human ABM-SC, however, both IL-13 and TNF-alpha were significantly reduced (P<0.001), suggesting that ABM-SC could be utilized therapeutically to treat chronic inflammatory disorders or graft rejection by reducing focal or serum levels of inflammatory mediators. See, FIGS. 25A, B, and C.

Notably, ABM-SC induced elevated expression of IL-2 in both autologous (Responders+Self) and allogeneic (Responders+Stimulators) mixed PBMC cultures (P<0.001) while simultaneously suppressing PBMC proliferation. While this result appears somewhat paradoxical given the importance of IL-2 in promoting T cell proliferation, recently it has been shown in mice that disruption of the IL-2 pathway results in lymphoid hyperplasia and autoimmunity rather than immune deficiency, suggesting that the major physiological role of IL-2 may be to limit or regulate, rather than enhance T cell responses (Nelson, “IL-2, Regulatory T-Cells, and Tolerance,” Jour. Immunol. 172: 3983-3988 (2004)). Additionally, it is now known that IL-2 is also critical for promoting self-tolerance by suppressing T cell responses in vivo and that the mechanism by which this occurs is through the expansion and maturation of CD4+/CD25+ regulatory T cells. It is, therefore, contemplated that ABM-SC could be employed therapeutically to induce T-cell tolerance by indirectly supporting the maturation of T regulatory cells through the induced up-regulation of IL-2.

### Example 19

### Human ABM-SC Inhibit Mitogen-Induced Peripheral Blood Mononuclear Cell Proliferation

Methods: Human adult bone-marrow derived somatic cells (ABM-SC) were cultured in vitro for 96 hours in a humidified incubator under 5% CO₂ then passaged onto 96-well round bottom plates at a concentration of 25,000 viable cells/mL in RPMI-complete media (HYCLONE™). Human peripheral blood mononuclear cells (PBMC) were cultured either separately at 250,000 cells/mL in RPMI-complete media, or with ABM-SC Lots RECB801 (sub-cultured to about 19 population doublings) or RECB906 (sub-cultured to about 43 population doublings). To stimulate PBMC proliferation, cultures were inoculated with 2.5 microg/mL phytohaemagglutinin (Sigma Chemical Co.). After 56 hours in culture, cells were pulsed with Thymidine-[Methyl-3H] (Perking Elmer, 1 microCi/well). Cells were harvested at 72 hours using a Filtermaster harvester onto glass filters. Filters were read in Omnifilter platers using an NXT TopCount Scintillation counter. Human mesenchymal stem cells were included as a positive control. (Human mesenchymal stem cells were obtained from Cambrex Research Bioproducts; now owned by Lonza Group, Ltd, Basel, Switzerland). Statistical analysis was performed by one-way ANOVA (analysis of variance).

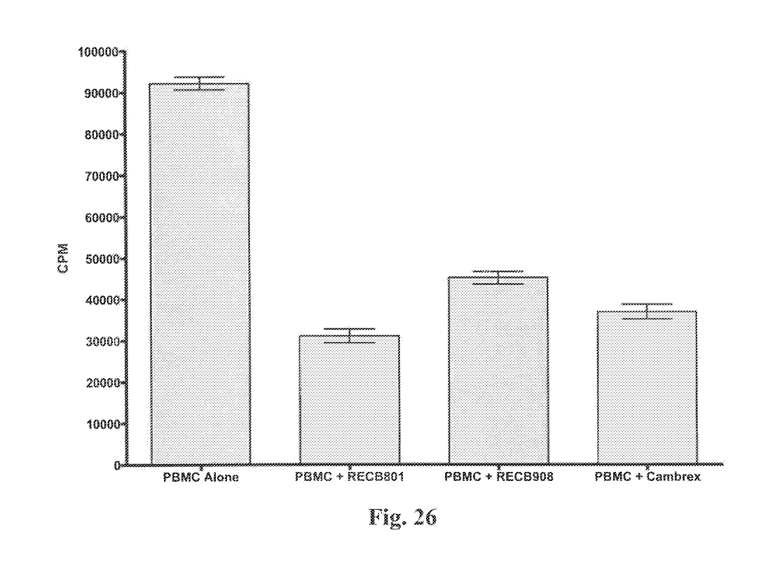

Results: PBMC-induced proliferation was significantly reduced when challenged with either lot of ABM-SC (P<0.001). See, FIG. 26. Mesenchymal stem cells (MSC) were included as a positive control. These data suggest that ABM-SC not only inhibit mitogen-induced proliferation of the total PBMC preparation, but that the presence of ABM-SC in this assay system does not induce proliferation of various cell subpopulations within the preparation (e.g., monocytes, granulocytes, lymophocytes).

### Example 20

### Collagen-Based, Bioactive Devices

The following abbreviations are used in this Example:

ADG, Media formulation based on Advanced DMEM with L-glutamine and HEPES
BSC, biosafety cabinet (laminar flow hood)
BSS, balanced salt solution
CFM-G, a cryopreservation medium containing MEM, glycerol, calf serum and FBS
DMEM, Dulbecco's modified eagles medium
DPBS, Dulbecco's phosphate-buffered saline
ELISA, enzyme-linked immunosorbent assay
EthD-1, ethidium bromide (red stain for dead cells)
Glut, glutaraldehyde
hABMSC(s), human adult bone marrow-derived stromal cell(s)

### HEPES,

PBS, phosphate-buffered saline
RPM, revolutions per minute
VEGF, vascular endothelial growth factor

### Introduction

Human adult bone marrow-derived stromal cells (hABM-SC) secrete a wide variety of factors involved in tissue repair and regeneration. When combined with rat tail collagen, these cells survive for a period of days, cause the construct to contract in a dose-dependent manner and release factors into the media (refer to study RND-04-032-3). The construct generated from combining and culturing hABM-SC and rat tail collagen is a pliable entity containing therapeutic factors that has the potential to be marketed as a medical device. This Example provides methods of preparing clinical grade, GMP collagen-based, bioactive medical devices.

Four major steps were involved in the production of collagen-based, bioactive devices: 1) cell preparation, 2) collagen gel formation, 3) culture of collagen plus cell constructs and 4) collagen construct processing. Each step contained a series of variables and opportunities for adjustment. Over 200 different devices with varying degrees of bioactivity, durability, flexibility, and size were created.

Increasing cell density, collagen concentration, and/or collagen gel volume resulted in higher bioactivity. In embodiments, a collagen concentration of either 4 mg/ml or 6 mg/ml resulted in optimal bioactivity in this study. Devices produced from collagen gel volumes up to 9 ml were feasible and resulted in higher levels of VEGF. However, the higher volume gels had reduced durability compared to other device iterations. Glutaraldehyde cross-linking concentrations used to process the constructs were optimal between 0.005%-0.05%. Dehydration on polyethylene plastic in a laminar flow hood resulted in a device that was thin, flexible, and able to be stored at room temperature.

Collagen-based, bioactive device s were successfully fabricated using hABM-SC and clinical grade porcine collagen. The following protocol provides methods of producing non-living, bioactive medical devices with relatively low COGs, durability and stability. In embodiments of the invention, the parameters identified as optimal include:

6e6 hABM-SC
3 or 4 days in culture
4 or 6 mg/ml collagen
6-9 ml gel volumes
0.005%, 0.01% and 0.005% glutaraldehyde.

### Objectives

The objectives of this study were to develop medical devices that 1) were devoid of living cells but retained bioactive factors 2) were amenable to long term storage and easy shipping and 3) could be scaled up for GMP manufacturing.

### Study Design

In this Example, production of collagen-based, bioactive devices using hABM-SCs involved 4 major steps as outlined in FIG. 40. Each step entails multiple components that can be altered to produce devices with different characteristics. This Example is a summary of many smaller experiments wherein components were altered in a step-wise fashion in order to identify the best combination for production of a non-living, bioactive, scale-able, medical device. A summary of the parameters that were altered is presented in Table 4.

TABLE 4
Parameters varied in creating multiple device iterations.
Living Construct Parameters:

| | |
|---|---|
| Gel Formulation: | multiple (change with collagen conc. and gel |
| | volume) |
| Collagen Concentration: | 2 mg/ml, 3 mg/ml, 4 mg/ml, 6 mg/ml, 8 mg/ml |
| Cell Number: | 3e6, 6e6, 7.5e6, 15e6, 18e6 |
| Gel Volume: | 2 ml, 3 ml, 4 ml, 5 ml, 6 ml, 7 ml, 8 ml, 9 ml |
| Culture Time: | 1, 2, 3, 6, 9 days |
| Feed Regimen: | no feed, 50% every second day, 100% every |
| | third day |
| Cell Preparation: | frozen, cultured |

Non-Living Construct Parameters:

| | |
|---|---|
| Xlink Concentration: | none, 0.0001%, 0.001%, 0.005%, 0.01%, |
| | 0.05%, 0.1%, 0.5% |
| Xlink Time: | 0.5 hr, 1 hr |
| Xlink Quench Time: | 1 hr, 2 hr, 4 hr, overnight |
| Wash Buffer: | none, PBS, DPBS, BSS, BSS + Dextrose |
| Dehydration Surface: | plastic, foil, plate |

Over 200 distinct combinations/devices were created. The devices were first evaluated by physical observations and measurements; size, texture, color, surface profile. Devices that were deemed acceptable by their physical features went on for further testing; quantification of factors by ELISA, mechanical testing, collagenase digestion.

### Materials and Methods

### Cells

Cell type: Product level hABMSCs (multiple lots were used throughout this study)

Culture vessel: none, cells used from frozen vials directly after thawing and resuspending

Seeding density: Seeding density within the collagen gel constructs was varied throughout these studies, but included total cell numbers in each device of 6e6, 7e6, 8e6, 10e6, 15e6 viable cells.

### Materials and Equipment

### Device Materials and Equipment:

Advanced DMEM with L-glutamine (ADG): Advanced DMEM, 4 mM glutamine, 20 mM HEPES
Collagen: TheraCol collagen from porcine skin 10 mg/ml (1%; Sewon Cellontech, Korea)
Collagen Buffer Solution (for 4 mg/ml collagen gels): 16 ml 7.5% sodium bicarbonate, 4 ml 1M HEPES, 2 ml 1N sodium hydroxide, 78 ml sterile water
Collagen Buffer Solution (for 6 mg/ml collagen gels): 20 ml 7.5% sodium bicarbonate, 6.66 ml 1M HEPES, 5.3 ml 1N sodium hydroxide, 68 ml sterile water
10×DMEM with L-glutamine: 10×DMEM, 10 mM L-glutamine working glutaraldehyde solutions: 8% glutaraldehyde stock solution diluted in 1×DPBS to final concentration
5M glycine solution: glycine powder (Sigma), 1×DPBS

### 1×DPBS

Suspension culture 6 well plates (35 mm diameter wells, Grenier BioOne)
Flat end spatulas
V. Mueller sterilization pouches 12″×15″ (polyethylene plastic)
Trypan Blue-Gibco cat#15250-061

### Biosafety Cabinet (BSC):

### Incubator: Form a 3150

### Centrifuge: Beckman Coulter Allegra 6R

### Hemacytomer: Brightline

### Invert Phase Microscope: Nikon Model TS100

### Device Testing Materials and Equipment:

calcein AM and EthD-1 stains (Live/Dead Cell Viability/Cytotoxicity Kit, Molecular Probes)
collagenase/hyaluronidase (Stern Cell Technologies Inc.)
balanced salt solution (BSS,)

### VEGF ELISA (VEGF ELISA Quantikine Kit, RnD Systems)

scissors
water bath at 37° C.
vortex

### Experimental Procedure

### Device Production

Four major steps were completed in the production of collagen-based, bioactive devices; 1) cell preparation, 2) collagen gel formation, 3) culture of collagen plus cell constructs and 4) collagen construct processing as outlined in FIG. 40 above.

In brief, constructs were formed by encapsulating hABM-SCs in a collagen gel solution. Six well suspension plates with a well diameter of 35 mm were filled with the cell containing gel solution and incubated at 37° C. for gelation. Once the gel solution had solidified it was detached from the well and cultured in suspension with media. The collagen gel constructs were cultured under low O₂ conditions, during which the cells actively contracted the collagen gels. At the end of the culture period, the constructs were processed by glutaraldehyde cross-linking followed by glycine washing. The construct was finally dehydrated rendering the cells inactive while preserving the bioactive factors secreted by the cells.

In each step, there was opportunity for variation. Wherein the process was modified or multiple iterations conducted details are provided.

### Cell Preparation

Most devices were made using hABM-SC thawed directly from vials stored under liquid nitrogen. For these constructs, the following steps were taken to prepare the cells:
1. Frozen vials were removed from liquid nitrogen tank and thawed in a 37° C. water bath for 6-10 mins.
2. Cells were resuspended with ADG media in 50 ml conical tubes.
3. Cells in conical tubes were centrifuged at 1,240 rpm for 5 mins to pellet cells.
4. Supernatants were removed and cells were resuspended again in ADG media for counting.
5. 100 ul sample of cell suspension was taken and diluted 1:10 in ADG media. Cell counting and viability was assessed by using this sample in a 1:1 dilution of trypan blue in a hemacytometer. Live cells were counted that excluded trypan blue and dead cells were counted that retained the dye to stain blue.
6. The final cell concentration was used to aliquot the appropriate number of viable cells into a single 50 ml conical tube.
7. Conical tubes with cell suspensions were again centrifuged at 1,240 rpm for 5 mins to pellet cells.
8. All supernatant liquid was removed from the cell pellet. The pellets were then ready for mixture with the collagen gel solution for construct formation.

### Collagen Gel Formation

Collagen gel constructs were formed by mixing the gel components together with the cell pellets. The components were always mixed in the following order: 10×DMEM with L-glut, collagen buffer solution, stock TheraCol collagen, and then mixture is added to resuspend the cell pellet.

Table 4 summarizes the different parameters used in generating the devices. Three different collagen buffer solutions were used for the 4 mg/ml, 6 mg/ml, or 8 mg/ml gels. All gel solution components were kept at 4° C. until they were combined with cells to initiate gel formation.
1. 10×DMEM with L-glutamine was added to either a 50 ml conical tube (if making 6 gels) or a 250 ml bottle (if making 12 gels). Volume of 10×DMEM for each construct was 1/10 the final gel construct volume to bring the DMEM to a 1× solution within the construct.
2. The collagen buffer solution for the appropriate 4, 6, or 8 mg/ml final collagen concentration gel was added to the 10×DMEM and swirled to mix.
3. The stock 10 mg/ml TheraCol solution was added by pipetting the collagen into the bottle while swirling with opposite hand to evenly distribute collagen throughout solution.
4. The components were rapidly mixed into a homogenous solution by quickly pipetting the solution up and down with the same pipette (collagen will coat the inside of the pipette, but will continue to flow out with pipetting up and down during mixing). The bottle containing the solution was also swirled during pipetting to aid in mixing.
5. The solution was fully mixed with the appearance of even pink color throughout combined with even consistency of viscosity.
6. The gel solution was pipetted onto the cell pellet and quickly pipetted up and down to thoroughly re-suspend the cells into the gel solution. Pipetting continued until the solution became evenly cloudy with cells resuspended and no visible signs of cell aggregates.
7. This cell suspension was then evenly dispensed throughout the rest of the collagen gel solution with repeated pipetting up and down to evenly distribute the cells throughout the gel solution.
8. A defined amount of cell plus collagen solution was pipetted into each well of the suspension culture 6 well plates. This final volume ranged from 3 ml to 9 ml as different devices were created and tested.
9. The culture plates containing the gel solutions were immediately placed into the humidified incubator at 37° C. with 5% CO₂ and 18% O₂. The plates remained undisturbed for 1 hour to complete gelation of collagen.

### Culture of Collagen and Cell Constructs

1. After 1 hour incubation, the plates were removed from the incubator and placed in the BSC.
2. Collagen gel constructs after complete gelation were lifted from the wells of the plates using a flat end sterile spatula. The spatula was inserted between the edge of the gel and wall of the well and cut around the circumference to completely pull the gel away from the wall of the well.
3. Using the spatula the gels were lifted from the bottom of the wells by gently pushing the edge toward the center along the circumference.
4. For 4, 5, 6, and 7 ml gel volume constructs, 6 ml of ADG media was added to each well containing the collagen gel constructs. For 8 and 9 ml gel volume constructs, 4 ml of ADG media was added.
5. Each construct was ensured to be freely floating within the media, or else a spatula was used to further lift the construct completely.
6. The culture plates were then placed in the low oxygen 4% O2 5% CO2 humidified incubator at 37° C. for culture.
7. Constructs were cultured from 1 day to 9 days. Most constructs were cultured for 64-72 hours.

### Collagen Construct Processing

After culture of the cells in the collagen gels, the constructs were processed to render a final non-living device. The processing included glutaraldehyde cross-linking, glutaraldehyde quenching and dehydration.

The glutaraldehyde cross-linking reaction was terminated under the condition of excess amine groups. The addition of a high concentration glycine solution allowed unreacted glutaraldehyde free ends to react with the glycine. This quenching step can prevent and limit the potential toxicity of using glutaraldehyde as the cross-linker.
1. Cultured constructs were cross-linked by the addition of 6 ml of glutaraldehyde solution. The gel constructs were kept in the original culture plates throughout the glutaraldehyde cross-linking and washing steps.
2. The cross-linking with glutaraldehyde was carried out for 30 mins or 1 hr at room temperature with slight movement on a plate shaker.
3. The glutaraldehyde solution was then removed from the wells and 6 ml of 1×DPBS was added to begin washing out of residual glutaraldehyde.
4. At least four total washes of 6 ml of 1×DPBS were added for 10 mins and removed from each well. For at least two of the washes the plates were placed on a plate shaker for gentle agitation.
5. The glutaraldehyde cross-linking reaction was quenched by the addition of 6 ml of 0.5M glycine solution to each well. Plates were placed on the plate shaker with gentle agitation during glycine quenching at room temperature for 2, 3, or 4 hours.
6. At the end of the glycine quenching, the solution was removed and the constructs were again thoroughly washed with DPBS for at least four total washes exactly as specified in step 4.
7. After the last wash, as much liquid was removed as possible from the well around the cross-linked construct to begin the dehydration of the constructs within the BSC.
8. A variety of dehydration surfaces were tested; tin foil, polyethylene plastic (specifically 12″×15″ polyethylene sterilization pouches), directly within the bottom of the tissue culture plate. These surfaces were placed within the BSC.
9. Each single cross-linked construct was transferred from the well plates to the dehydration surface with a flat end spatula.
10. The constructs were spaced at least 10 cm from each other. Constructs were left in the BSC overnight with the hood light turned off, the blower remaining on and the hood door open to its appropriate functioning height.
11. The constructs dehydrate into a very thin paper-like disk after dehydration overnight in the BSC producing the device configuration that was used in further test experiments.

### Device Testing

Device testing was performed according to methods described herein. The following tests were performed: evaluation of Physical Parameters, Collagenase Digestion and Bioactivity with VEGF ELISA

### Results

### Variation of Living Construct Parameters

For these experiments, the focus was on optimizing culture conditions that would increase the quantity and capture of factors secreted from the hABM-SCs within the living collagen gel constructs. Initial methods included varying density, concentration of the collagen gel and culture time of cells within the collagen gels.

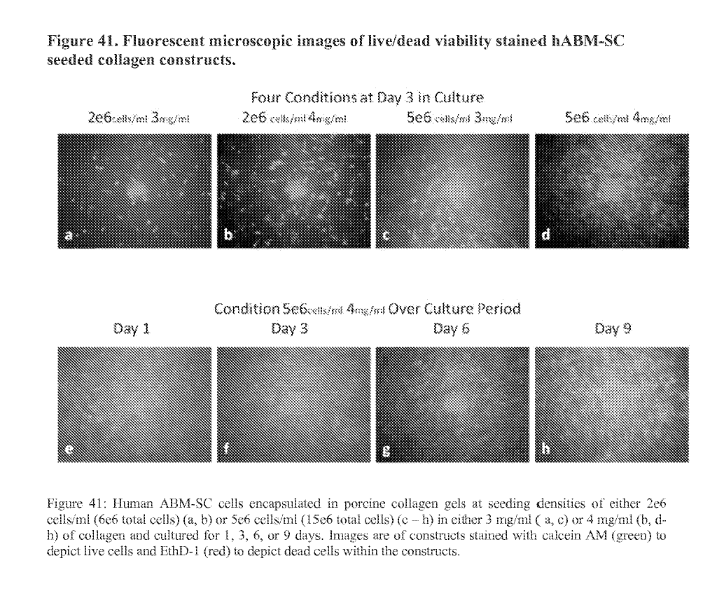

Based on previous experiments and taking into consideration COGs, cell densities of 2e6 cells/ml and 5e6 cells/ml were explored in these studies. Constructs made for initial experiments utilized collagen gels that were 3 ml in volume, so the final total cell numbers were either 6e6 or 15e6 cells in each gel. The collagen gel concentrations were either 3 mg/ml or 4 mg/ml (3 ml volume of each gel). The third variable in this initial study was culturing the hABM-SC seeded collagen gels for 1, 3, 6, or 9 days under low oxygen tension of 4% O₂, 5% CO₂, buffered with N₂ at 37° C. FIGS. 41 & 42 represent the results of this initial study with analysis of cell viability, cell morphology, cell activity, and construct bioactivity.

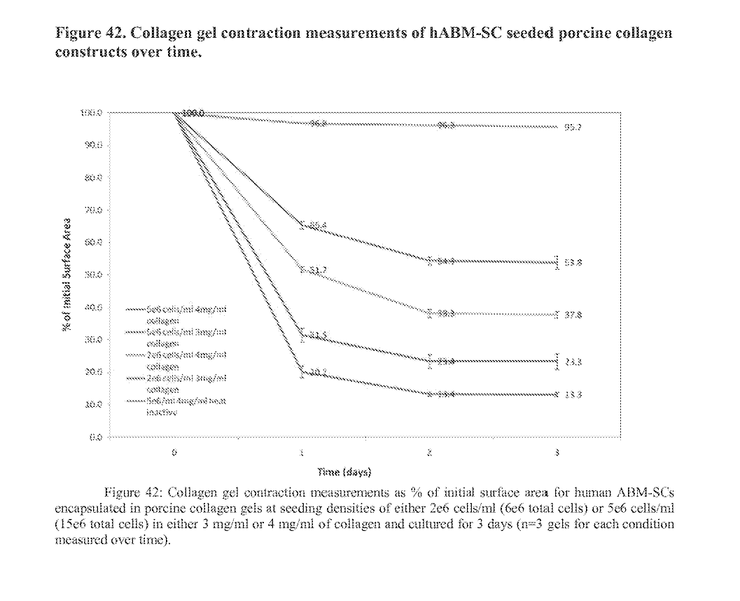

FIG. 41 represents images of cell viability (calcein AM in green=live, EthD-1 in red=dead) staining of living constructs from the first studies completed. The top row of images in FIG. 41 highlight the difference in cell seeding density and collagen concentration on cell morphology between four devices after 3 days in culture. Constructs seeded with a higher concentration of collagen appear to have more live cells. Cell death in the 3 mg/ml constructs was also slightly elevated, observed by more EthD-1 red staining. It also appears that the collagen gels can tolerate and maintain up to 5 million ABM-SC per 1 ml of collagen gel. Because constructs with higher collagen contract more and therefore are more dense (refer to FIG. 42 for evidence), it is possible that the appearance of more viable cells in the 4 mg/ml 5e6/ml devices is actually just due to reduced gel size and increased overall density.

In the bottom row of FIG. 41, devices seeded with the same number of cells but cultured for 1 to 9 days highlight the impact of culture time. Within the first three days of culture of the cell-seeded collagen constructs the cells manipulate the gel and contract it to a smaller volume. The morphology at Day 3 (FIG. 20 shows some alignment of the cells with their contraction of the gel. Morphologies on Day 3 to Day 6 are similar, but by Day 9 in culture more dead cells appear within the constructs (red staining).

It appears that a higher cell number and increased collagen concentration is preferable for maintaining more, viable, contracting cells. In theory more viable cells should result in more bioactive factors.

As shown in FIG. 42 both increased cell density and collagen content resulted in greater contraction of constructs. Devices with the 5e6 ABM-SC contracted faster and to a great degree than those with 2e6 ABM-SC. When collagen content was increased to 4 mg/ml, constructs contracted faster and to a greater degree. When combined, the effect was additive.

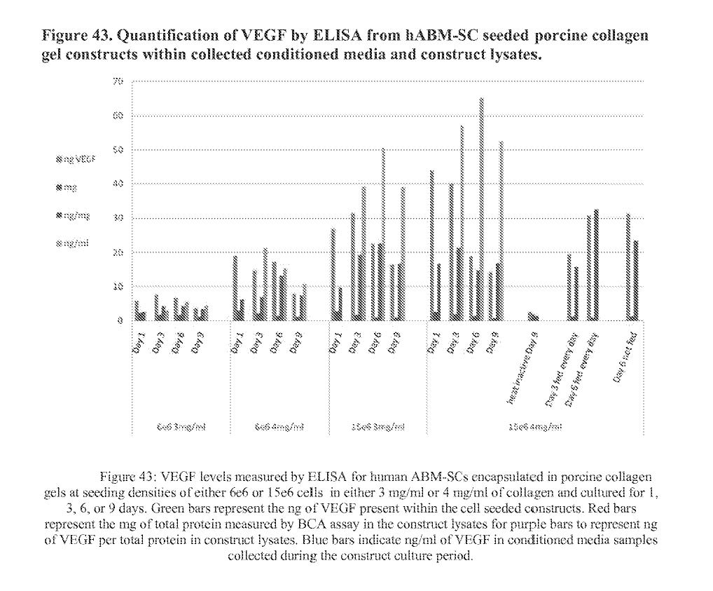

To assess bioactivity of these preliminary constructs, VEGF within the devices and secreted into the culture media was quantified using an ELISA and is summarized in FIG. 43. VEGF contained within the constructs was determined by digesting the device, analyzing a portion of this solution and then calculating back for total content (green bars). Total protein content of the devices was determined using a BCA kit (red bars). The quantity of VEGF per device was normalized to total protein content (purple bars). For analysis of secreted factors, the culture media was collected at the end of the experiment, analyzed and represented as amount of factor per ml of culture media (blue bars). For some constructs, the supernatant was not analyzed and therefore, the blue bar is missing from that set of data.

The ELISA data summarized in FIG. 43 indicates that 1) adding more cells (15e6 vs 6e6) results in more VEGF per construct, 2) increasing collagen content in constructs (3 mg/ml to 4 mg/ml) results in more VEGF per construct, and 3) culturing devices more than 6 days leads to reduced VEGF content with more instead released into the culture media.

Also, results of FIG. 43 demonstrated that a feed protocol for a construct cultured for 3 days was best to have no change in media of this time period, due to a decrease in VEGF content when the construct had a media change every day. For an extended 6 day cultured construct, the feed protocol of having a media change every day or not changing the media at all were similar in resultant VEGF content, but VEGF content was reduced when the construct had a single media change on day 3. Therefore, optimal VEGF content within the construct can be achieved by not changing the culture media at all over any culture time under 6 days. This is significant in lowering both the cost of goods associated with media changes and reducing manufacturing personnel time and costs.

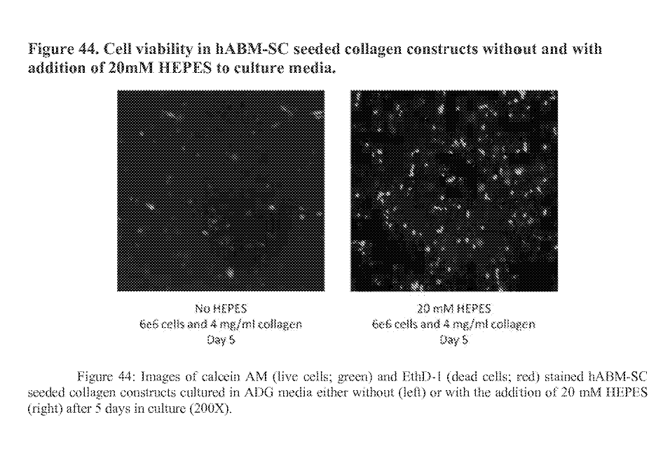

Constructs that were cultured within the same media throughout the entire culture period had maximal VEGF contained within the device. However, while culturing constructs for 6 days without feeding is beneficial for maintaining high VEGF content, the culture media over this time does become slightly acidic. In order to improve the culture conditions, addition of HEPES to the media was tested with the results shown in FIG. 44.

Addition of 20 mM HEPES to the culture media improved survival of cells in collagen gel constructs during the culture period as seen in FIG. 44. Constructs cultured with 20 mM HEPES in the media had many more viable (green cells) and less dead cells (red); compare the right panel to the left in FIG. 44. Therefore, all subsequent experiments included the addition of 20 mM HEPES to the Advanced DMEM with L-glutamine media for culture of the constructs.

The following observations were made during these initial studies:
1. Increased cell seeding led to increased gel contraction, VEGF secretion and content.
2. Higher concentration of collagen led to increased gel contraction, VEGF secretion, and VEGF capture within the construct.
3. Construct culture times of 1 and 3 days maximized the capture of VEGF contained within the constructs more so than longer culture times.

Based on these results constructs with 4 mg/ml collagen concentration or higher, cultured for less than 6 days were pursued for the next studies.

The focus during this next phase was to prepare N=3 of a few lead devices for a more stringent analysis and comparison. The focus was on cell density, culture time and feeding protocol. All constructs were made at the higher collagen concentration of 4 mg/ml. Culture times of 1, 3, or 6 days were considered with 6 day cultures fed either once on day 3 or not at all. The results from quantification of VEGF content are presented in FIG. 45.

When comparing 6 day cultures that were fed to those not fed, a clear trend emerged. Not feeding devices cultured for 6 days resulted in enhance VEGF levels within the constructs. However, the constructs with maximal VEGF were those cultured for 3 days. As seen previously, increasing the number of cells seeded (6 vs 15 vs 18) correlated with increases in the VEGF levels.

Based on all of the above data it was decided that culture times would be kept to 4 days or less and collagen content would be locked in at 4 mg/ml. Due to limited resources of ABM-SC and the impact on cost, it was also determined that cell number would be locked at 6e6 ABM-SC per construct.

a) Variation of Parameters with Non-Living Construct Processing

The goal of the next phase was to process the living constructs to create a non-living device that is durable and pliable, a final product able to endure storage at room temperature and be handled by surgeons during application. Importantly, the techniques used during processing must preserve or not significantly reduce the bioactive factors secreted by the hABM-SCs during the culture of the constructs.

Glutaraldehyde cross-linking of the constructs was chosen as the most acceptable way to produce a more durable product due to the simple cross-linking protocol required and other prior FDA approved collagen products utilizing this cross-linker (i.e. Zyderm and Zyplast). Dehydration after cross-linking was chosen as the method to reduce the device into a thin flexible dry material able to be stored at room temperature. The results presented within this section summarize the modifications tested for final processing. From here on in this Example, the term device refers to the non-living constructs that have undergone processing with glutaraldehyde cross-linking and dehydration to result in a final product. The term construct will refer to the living cell seeded collagen gel construct prior to processing.

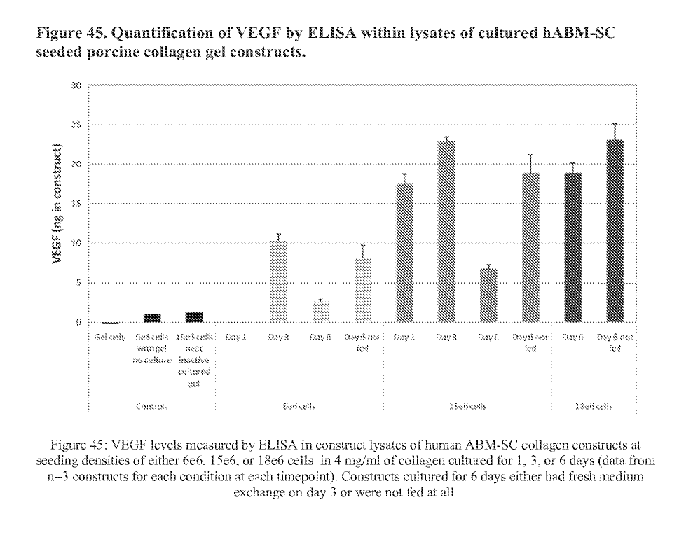

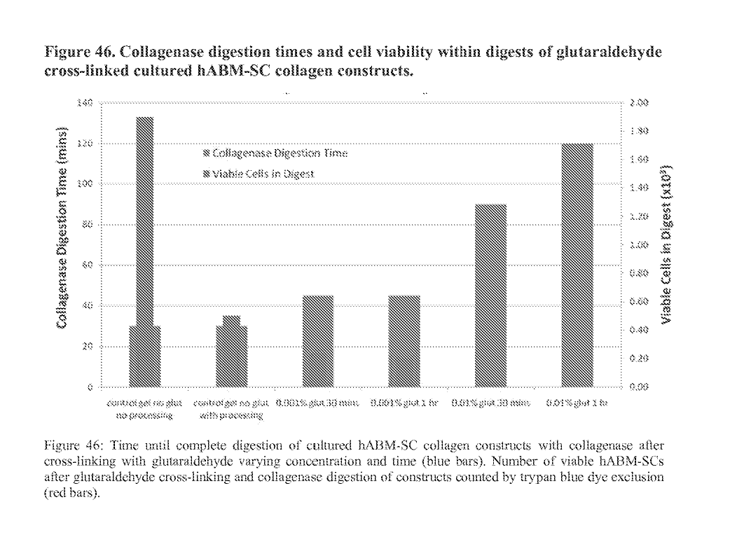

The first experiments looked at the impact of glutaraldehyde (glut) concentration and cross-linking time on construct digestion and cell viability. The results of these first processed “non-living” iterations are shown in FIG. 46.

An increase in glut concentration or cross-linking time increased the construct's resistance to digestion with collagenase and reduced cell viability, as shown in FIG. 46. Increasing the glut concentration and not the cross-linking time, proved to be the more effective method to improving cross-linking of the constructs. All of the cross-linking protocols resulted in collagen constructs that were much more durable to handle. Glut fixed devices maintained their integrity, unlike the unprocessed constructs that could easily collapse and fall apart upon first handling.

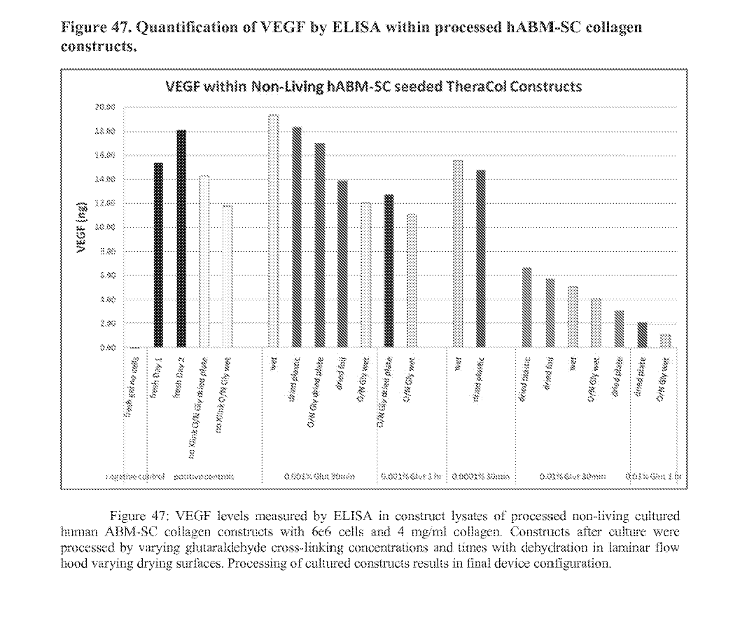

While the fixation protocol was continuing to be optimized, methods for dehydration were incorporated. In the absence of access to a vacuum drier and anticipating that one would not be available during GMP manufacturing, air drying in a biosafety cabinet was utilized. Drying surfaces tested included foil, thin flexible polyethylene plastic (further referred to as plastic), and the tissue culture plate used to culture the constructs. FIG. 47 displays the results of the initial study testing different surfaces as well as further variation of the cross-linking conditions and their effects on device VEGF levels.

Dehydration on the plastic surface helped preserve the most VEGF during the dehydration period compared to other surfaces. Unfortunately, dehydration directly in the tissue culture plate resulted in difficulty removing the dehydrated device making it an impractical surface for future manufacturing processing. Devices dehydrated on the cellophane and foil surfaces allowed a non-stick surface in which the devices were easily peeled off at the end of the dehydration period. Therefore, all continued development of the devices included dehydration of the constructs on the polyethylene plastic surfaces.

The results in FIG. 47 re-confirm the initial observation that increased glut results in reduction of VEGF content or recovery (observe the trend from left to right). As well, longer fixation time (compare 30 min to 1 hr) resulted in decreased VEGF content or recovery. Cross-linking constructs with glut of concentration of 0.001% for 30 min and dehydrated on plastic resulted in a decrease of VEGF under only 2 ng down from the non-crosslinked unprocessed construct.

Considering all the data collected, it was determined that this processing method, glut with glycine washing and dehydration, was most feasible to produce a cross-linked dehydrated device that preserves sufficient VEGF. The resultant device was a very thin paper-like material that was pliable and significantly more durable for handling compared to an unprocessed construct. The processed construct was also more stable in the dehydrated state allowing the devices to be stored long term at room temperature.

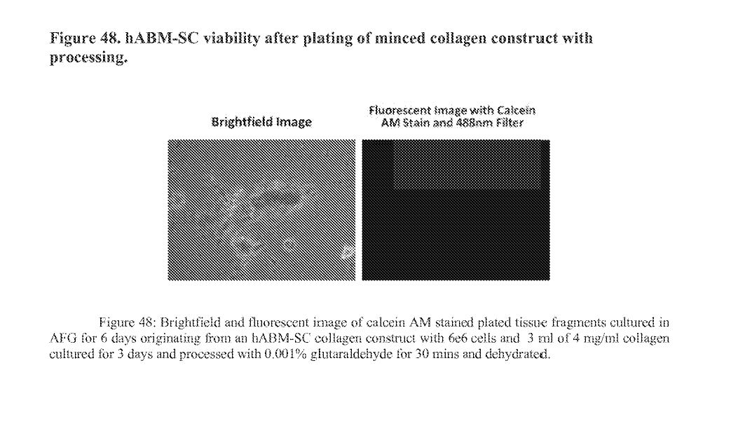

To assess cell viability after complete processing including glut cross-linking and dehydration, a device (6e6 cells, 3 ml gel volume, 4 mg/ml collagen, cultured for 3 days, processed with 0.001% glut for 30 mins, dehydrated) was minced with scissors to pieces less than 1 mm³ and plated in AFG (hABM-SC growth media with 10% FBS) for 6 days. This culture was monitored daily to observe any plating and/or expansion of hABM-SCs from the processed device. Brightfield and fluorescent images from the culture are presented in FIG. 48.

Debris and cell remnants were present within the culture, but no change over the culture period was observed to indicate expansion of viable cells originating from the device. After 6 days in culture the debris was stained with calcein AM dye, which is actively taken up by only viable living cells and stains them green. The brightfield image in FIG. 48 highlights a device/cell debris cluster present after 6 days in culture. This cluster did not stain positively with the calcein dye, proving there were no viable cells within the cluster. No positive green staining indicating any viable cells was seen throughout the entire culture and all debris. These results indicate that the processing of the living construct with 0.001% of glutaraldehyde for 30 mins with dehydration appears to render the device non-living or devoid of live cells.

To assess the stability of the dehydrated constructs, the VEGF content of several devices that had been maintained at room temperature for several days was analyzed. This study also included a device that was fixed using ethanol, pre-processed constructs that were washed with different buffers and devices that were generated with more than 3 mls of collagen. Two methods of increasing the collagen content were tested; increasing collagen concentration to 6 mg/ml or increasing the volume of the construct from 3 ml of collagen solution to 6 ml.

Increasing the collagen concentration within this experiment resulted in no increase of VEGF contained within the device, but doubling the gel volume resulted in doubling the quantity of VEGF. The doubling of the collagen gel volume resulted in the most outstanding improvement in maximizing the quantity of VEGF contained within the device. Increasing the gel volume had a comparable impact on VEGF content to increasing the cell number seeded from 6e6 to 15e6. This was a remarkable finding because the cost of goods in increasing the amount of porcine collagen used to produce the devices is significantly less expensive than increasing the number of hABM-SCs required for each device.

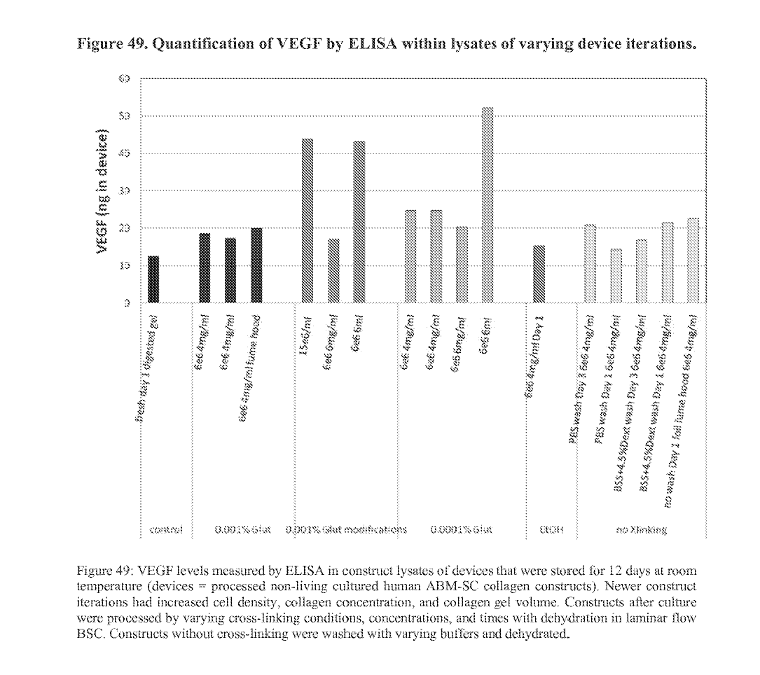

Continued experiments relied on achieving higher VEGF levels with the collagen gel constructs at the 6 ml volumes. The iteration within FIG. 49 with the highest level of VEGF at 52 ng was the device with 6e6 cells at 4 mg/ml collagen of a 6 ml volume cultured for 3 days and cross-linked with 0.0001% glut for 30 mins and dehydrated. This iteration maximized the VEGF level, however, the durability of this device was not acceptable. This very low level of cross-linking, 0.0001% glutaraldehyde, was not able to endure moderate handling while maintaining its integrity. After handling, the device easily collapsed on itself and became deformed. Therefore, it was concluded that a cross-linking concentration of glut at or higher than 0.001% was necessary to produce devices that were durable enough to withstand handling necessary during patient application. This would, however, compromise the bioactivity of the device.

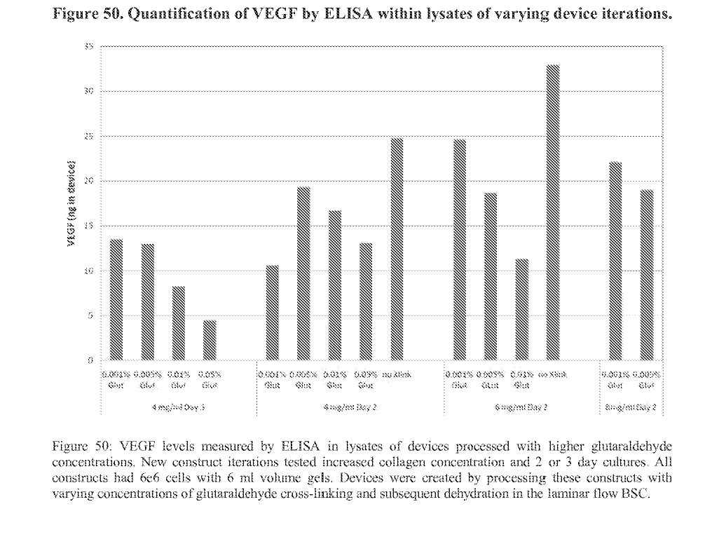

Constructs with 4 mg/ml collagen cultured for 2 days resulted in VEGF levels comparable to 3 days of culture. The most significant result within the iterations of FIG. 50 was that increasing the collagen concentration to 6 mg/ml at a cross-linking concentration of 0.001% glut maximized the VEGF level compared to other iterations. Increasing the collagen to 8 mg/ml did not further increase the VEGF. The results in FIG. 48 of the first construct produced with a collagen concentration of 6 mg/ml resulted in no increase of VEGF compared to its counterpart device with 4 mg/ml of collagen because the collagen gel solution formulation was not optimized for the 6 mg/ml of collagen. The devices created for the results of FIG. 50 were after the gel formulation of 6 mg/ml collagen was changed to optimize the ability of the gel to set and contract during the culture period. Therefore, with the right gel formulation the 6 mg/ml collagen constructs did enhance the VEGF capture within the device compared with 4 mg/ml and 8 mg/ml iterations.

The glut concentration of 0.05% was ruled out due to the decreased pliability and increased stiffness with handling of the device. Glut concentrations selected for future devices included 0.001%, 0.005%, and 0.01%.

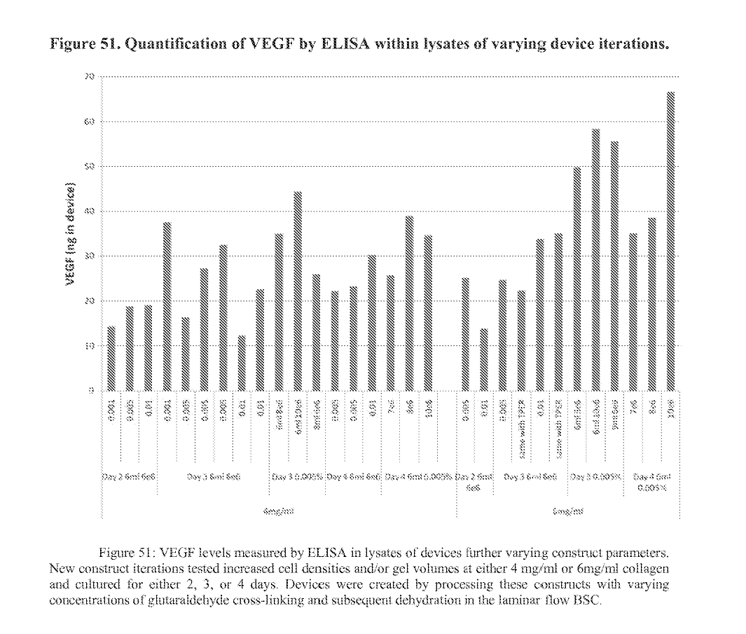

Another set of devices were prepared to compare the 4 mg/ml constructs with the results presented in FIG. 51. This data displays all possible iterations that were contenders for further development up until this point in time. Multiple iterations were performed in order to finally narrow the modifications to under ten device iterations. One modification included within these results was to observe the effects of increasing cell number only slightly to above 6e6 (instead of the previous leap from 6e6 to 15e6 cells).

### Discussion

Collagen-based, bioactive devices can successfully be fabricated using hABM-SC and clinical grade porcine collagen. Multiple devices with varying cell density, collagen concentration, collagen gel volume, culture time, glutaraldehyde cross-linking concentration and time, glycine quenching time, wash buffers, and dehydration surface were created and tested.

The studies showed that increasing cell density could significantly increase the quantity of VEGF contained within a device. However, adding more cells significantly drives up cost and therefore it was determined that devices would contain no more than 6e6 cells/device and other methods for elevating VEGF content would be pursued.

The most significant finding among these studies was that raising the collagen content of the constructs, by either concentration or volume, could increase the bioactivity. The increased collagen within a construct most likely contributed to both increased activity of the cells to secrete more factors and the ability of the gel to better retain these factors within the construct. After testing a wide range of collagen concentrations and volumes, it was noted that going higher than 6 mg/ml did not afford substantial increases in VEGF content. As a result, collagen at 6 mg/ml was selected as the optimal concentration used in further device development. Increasing the collagen gel volume above 6 ml seemed to decrease the strength of the device, but these iterations were still considered for further testing and development as they provided other benefits, such as higher VEGF content.

Processing of the cultured cell-seeded collagen constructs by glutaraldehyde cross-linking and dehydration resulted in a device containing no detectable viable cells or cells able to expand further in culture. Increasing the glutaraldehyde concentration resulted in a more durable construct, but concentrations above 0.05% decreased the flexibility of the resultant device. Glut concentrations further considered were 0.005%, 0.01%, and 0.05%.

To keep the overall processing time shorter and because longer times negatively impacted bioactivity, a cross-linking time of 30 mins was selected for future device iterations. The dehydration surface of polyethylene plastic proved superior over tin foil and the culture dishes. The polyethylene could be sterilized and allowed a non-stick surface in which the devices were easily removable after dehydration.

In an effort to preserve as much VEGF content as possible, final processing protocols included no washing of the cultured constructs before glutaraldehyde cross-linking and only washing after cross-linking with DPBS.

### Example 21

### Collagen-Based, Bioactive Devices

### Assessment of Devices for Hand Tendon Repair

The abbreviations in Example 20 are also used in this Example.

### Abstract

For hand tendon repair, the device needed to 1) be strong enough to tolerate suturing to itself or recipient tissue, 2) contain relevant bioactive factors, 3) tolerate handling by surgeons and 4) be thin and flexible.

Collagen-based bioactive devices fulfilling the preliminary criteria of strength, bioactivity, appearance, feasibility for scale-up GMP manufacturing and product distribution were successfully and reproducibly generated using hABM-SC as described herein. Six devices were determined to have the necessary characteristics to go-on for further testing by hand surgeons. These devices vary in dimension, strength and bioactivity while maintaining the core requirements for manufacturing and therapeutic application.

Elements to the devices presented are 1) the use of GMP-grade materials, 2) methods of production that can be scaled up for manufacturing and 3) physical and functional characteristics that satisfy the end-user: hand surgeons. The final six devices were thin and flexible. They were be easily manipulated and repeatedly handled upon rehydration. VEGF, used as a surrogate marker for bioactivity, was measured at nanogram levels in all devices. As well, all devices can withstand the suture retention test, holding several grams of weight before reaching load failure.

### Objective

The objectives of this study were to 1) use GMP materials and methods to create collagen-based, bioactive devices for hand tendon repair and 2) assess the physical and functional characteristics of these devices.

### Study Design

In this study elements in various combinations were altered to create a series of devices that fulfilled both manufacturing and clinical criteria. For manufacturing the requirements included; 1) device made from GMP-grade materials, 2) process that could accommodate scale-up and 3) device which could be stored at room temperature and have potential for long term stability. Preferred devices have the following features; 1) very thin, 2) flexible, 3) easy to manipulate 4) strong enough to hold a suture and 5) large enough to be cut to their desired size and shape.

Based upon the properties of the devices, the feasibility for large scale manufacturing, a subset of devices were advanced for additional testing. This report summarizes the methods of making and analysis of the subset that was selected for further consideration.

### Materials and Methods

### Cells

Cell type: hABMSC Lot # P15-T2S1F1-5, approx. 50e6 cells per vial
Culture vessel: none, cells used from frozen vials directly after thawing and resuspending
Seeding density: 6e6 viable cells seeded in each collagen gel construct

### Materials and Equipment

### Device Materials and Equipment:

Advanced DMEM with L-glutamine (ADG): Advanced DMEM, 4 mM glutamine, 20 mM HEPES
Collagen: TheraCol 1% collagen from porcine skin 10 mg/ml (Sewon Cellontech, Korea)
Collagen Buffer Solution (for 4 mg/ml collagen gels): 16 ml 7.5% sodium bicarbonate, 4 ml 1M HEPES, 2 ml 1N sodium hydroxide, 78 ml sterile water
Collagen Buffer Solution (for 6 mg/ml collagen gels): 20 ml 7.5% sodium bicarbonate, 6.66 ml 1M HEPES, 5.3 ml 1N sodium hydroxide, 68 ml sterile water
10×DMEM with L-glutamine: 10×DMEM, 10 mM L-glutamine
0.005% or 0.01% working glutaraldehyde solution: 8% glutaraldehyde stock solution, 1×DPBS
5M glycine solution: glycine powder (Sigma), 1×DPBS

### 1×DPBS

Suspension culture 6 well plates (35 mm diameter wells, Grenier BioOne)
Flat end spatulas
V. Mueller sterilization pouches 12″×15″ (polyethylene plastic)
Trypan Blue-Gibco cat#15250-061

### Biosafety Cabinet (BSC):

### Incubator: Form a 3150

### Centrifuge: Beckman Coulter Allegra 6R

### Hemacytomer: Brightline

### Invert Phase Microscope: Nikon Model TS100

### Device Testing Materials and Equipment:

calcein AM and EthD-1 stains (Live/Dead Cell Viability/Cytotoxicity Kit, Molecular Probes)
collagenase/hyaluronidase (Stem Cell Technologies Inc.)
balanced salt solution (BSS,)

### VEGF ELISA (VEGF ELISA Quantikine Kit, RnD Systems)

### Balance

mechanical testing apparatus (two lab ring stands with clamps holding bar with small clamp to secure device)
3-0 Ethibond suture (Ethicon)
weight basket (gauze with paper clip and staples)
weights (5 g, 10 g, 20 g)
scissors
water bath at 37° C.
vortex

### Experimental Procedure

### Device Production

In embodiments of the invention, four major steps are involved in the production of a collagen-based, bioactive device; 1) cell preparation, 2) collagen gel formation, 3) culture of collagen plus cell constructs and 4) collagen construct processing.

In brief, constructs are formed by encapsulating hABM-SCs in a collagen gel solution. Six well plates with a well diameter of 35 mm are filled with the cell containing gel solution and incubated. Once the gel solution has solidified it is detached from the well and cultured in suspension with media. The collagen gel constructs are cultured under low O2 conditions for a period of approximately 3 days, during which the cells actively contract the collagen gels. At the end of the culture period, the constructs are processed by glutaraldehyde cross-linking, glycine quenching to stop glutaraldehyde reaction, and dehydration rendering the cells inactive while preserving the molecules secreted by the cells.

In each step, there is opportunity for variation. Where-in the process was modified or multiple iterations conducted details are provided.

### Cell Preparation

Most devices were made using hABM-SC taken from vials stored under liquid nitrogen. For these constructs, the following steps were taken to prepare the cells:
1. Frozen vials at 50e6 hABMSCs/vial were removed from liquid nitrogen tank and thawed in a 37° C. water bath for 6-10 mins.
2. Cells were resuspended with ADG media in 50 ml conical tubes.
3. Cells in conical tubes were centrifuged at 1,240 rpm for 5 mins to pellet cells.
4. Supernatants were removed and cells were resuspended again in ADG media for counting.
5. 100 ul sample of cell suspension was diluted 1:10 in ADG media. Cell counting and viability was assessed by using this sample in a 1:1 dilution of trypan blue in a hemacytometer. Live cells were counted that excluded trypan blue and dead cells were counted that retained the dye to stain blue.
6. The final cell concentration was used to aliquot either 36e6 or 72e6 total viable cells into a single 50 ml conical tube. 36e6 cells were used if making a batch of 6 gel constructs and 72e6 for making a batch of 12 gels.
7. Conical tubes with cell suspensions were again centrifuged at 1,240 rpm for 5 mins to pellet cells.
8. All supernatant liquid was removed from cell pellet. The pellets were not resuspended yet.

For the one device made with cultured cells, a vial was thawed, hABM-SC were plated in T-flasks and incubated overnight at 2.2e4 cells/cm². The following day cells were harvested, washed and re-suspended in ADG media. The harvested cells were then processed in the same manner starting with Step 5 above.

### Collagen Gel Formation:

Collagen gel constructs were made by mixing the gel component together with the cell pellets. The components were always mixed in the following order: 10×DMEM with L-glut, collagen buffer solution, stock TheraCol collagen, and then cell pellet.

Table 5 summarizes the components and proportions used in generating the different constructs discussed in this study. Two different collagen buffer solutions were used for the 4 mg/ml or 6 mg/ml gels. All gel solution components were kept at 4° C. until they were combined with cells to initiate gel formation.
1. 10×DMEM with L-glutamine was added to either a 50 ml conical tube (if making 6 gels) or a 250 ml bottle (if making 12 gels).
2. The collagen buffer solution for the appropriate 4 or 6 mg/ml final collagen concentration gel was added to the 10×DMEM and swirled to mix.
3. The stock 10 mg/ml TheraCol solution was added by pipetting the collagen into the bottle while swirling with opposite hand to evenly distribute collagen throughout solution.
4. The components were rapidly mixed into a homogenous solution by quickly pipetting the solution up and down with the same pipette (collagen will coat the inside of the pipette, but will continue to flow out with pipetting up and down during mixing). The bottle containing the solution was also swirled during pipetting to aid in mixing. At least 2 ml of the solution was always kept in the pipette tip without fully dispensing all solution in order to prevent introduction of bubbles into solution.
5. The solution was fully mixed until the appearance of an even pink color throughout, combined with even consistency of viscosity.
6. 10 ml of the gel solution was pipetted onto the cell pellet and quickly pipetted up and down to thoroughly re-suspend the cells into the gel solution. Pipetting continued until the solution became evenly cloudy with cells resuspended and no visible signs of aggregates of cells.
7. This cell suspension was then evenly dispensed throughout the rest of the collagen gel solution with repeated pipetting up and down to evenly distribute the cells throughout the gel solution. At least 2 ml were always kept within the pipette tip in order to avoid introduction of bubbles.
8. 4-9 milliliters of cell plus collagen solution was pipetted into each well of the suspension culture 6 well plates.

The culture plates containing the gel solutions were immediately placed into the humidified incubator at 37° C. with 5% CO₂ and 18% O₂. The plates remained undisturbed for 1 hour to complete gelation of collagen.

TABLE 5
Amount of each component added together to make collagen gel construct.
Amount of each component added together to make collagen gel construct:

| | Final Collagen Concentration: | | | | | | |
|---|---|---|---|---|---|---|---|
| Gel Component | 4 mg/ml | 6 mg/ml | | | | | |
| 10X DMEM with L-glut | 0.6 ml | 0.4 ml | 0.5 ml | 0.6 ml | 0.7 ml | 0.8 ml | 0.9 ml |
| Collagen Gel Buffer* | 3 ml | 2 ml | 1.5 ml | 1.8 ml | 2.1 ml | 2.4 ml | 2.7 ml |
| stock TheraCol Collagen | 2.4 ml | 1.6 ml | 3 ml | 3.6 ml | 4.2 ml | 4.8 ml | 5.4 ml |
| Total Gel Volume | 6 ml | 4 ml | 5 ml | 6 ml | 7 ml | 8 ml | 9 ml |

There are two separate collagen gel buffer formulations for either 4 or 6 mg/ml gels.

Final parameters of collagen gel constructs:
6e6 cells/construct
Collagen concentration: 4 mg/ml or 6 mg/ml
Varying gel volumes: 4 ml, 5 ml, 6 ml, 7 ml, 8 ml, or 9 ml

### 1 mM L-glutamine

### 1×DMEM

0.45% sodium bicarbonate
15.9 mM sodium hydroxide

20 M HEPES

### Culture of Collagen Plus Cell Constructs

1. After 1 hour incubation, the plates were removed from the incubator and placed in the BSC.
2. Collagen gel constructs after complete gelation were lifted from the wells of the plates using a flat end sterile spatula. The spatula was inserted between the edge of the gel and wall of the well and cut around the circumference to completely pull the gel away from the wall of the well.
3. Using the spatula the gels were lifted from the bottom of the wells by gently pushing the edge toward the center along the circumference.
4. For 4, 5, 6, and 7 ml gel volume constructs, 6 ml of ADG media was added to each well containing the collagen gel constructs. For 8 and 9 ml gel volume constructs, 4 ml of ADG media was added.
5. Each construct was tested to ensure it was freely floating within the media, or else a spatula was used to further lift the construct completely.
6. The culture plates were then placed in the low oxygen 4% O2 5% CO2 humidified incubator at 37° C. for culture.
7. Most constructs were cultured for 3 days (between 64-72 hours). One iteration was cultured for 2 days (between 40-48 hours).

### Collagen Construct Processing:

After culture of the cells in the collagen gels, the constructs were processed to render a final non-living device. The processing includes glutaraldehyde cross-linking, glutaraldehyde quenching and dehydration.

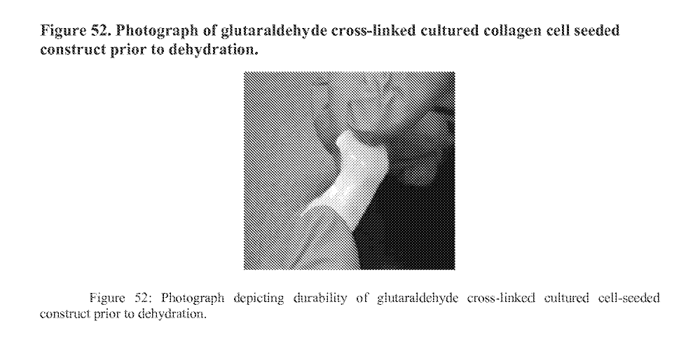

The glutaraldehyde cross-linking reaction is terminated under the condition of excess amine groups. The addition of high concentration glycine solution allows unreacted glutaraldehyde free ends to react with the glycine. This quenching step can prevent and limit the potential toxicity of using glutaraldehyde as the cross-linker.
1. Constructs cultured for 2-3 days were cross-linked by the addition of 6 ml of the appropriate working solution of glutaraladehyde; 0.005%, 0.01%, or 0.05%. The gel constructs were kept in the original culture plates throughout the glutaraldehyde cross-linking and washing steps.
2. The cross-linking with glutaraldehyde was carried out for 30 mins at room temperature with slight movement on a plate shaker.
3. After 30 mins, the glutaraldehyde solution was removed from the wells and 6 ml of 1×DPBS was added to begin washing out of residual glutaraldehyde.
4. At least four total washes of 6 ml of 1×DPBS were added for 10 mins and removed from each well. For at least two of the washes the plates were placed on a plate shaker for gentle agitation.
5. The glutaraldehyde cross-linking reaction was quenched by the addition of 6 ml of 0.5M glycine solution to each well. Plates were placed on the plate shaker with gentle agitation during glycine quenching at room temperature for 2, 3, or 4 hours. The effects of 2 and 4 hour quenching time were tested on the viability of cells cultured on these devices (results shown in FIG. 52).
6. At the end of the glycine quenching, the solution was removed and the constructs were again thoroughly washed with DPBS for at least four total washes exactly as specified in step 4.
7. After the last wash, as much liquid was removed as possible from the well around the cross-linked construct to begin the dehydration of the constructs within the BSC.
8. A 12″×15″ polyethylene sterilization pouch was placed in the BSC with the polyethylene plastic side facing up.
9. Each single cross-linked construct was transferred from the well plates to the plastic surface with a flat end spatula.
10. The constructs were spaced at least 10 cm from each other. Constructs were left in the BSC overnight with the hood light turned off, the blower remaining on and the hood door open to its appropriate functioning height.
11. The constructs dehydrate into a very thin paper-like disk after dehydration overnight in the BSC producing the device configuration that was used in further test experiments.

### Device Nomenclature

Each device was given a 4-5 digit number. The first digit corresponds to the collagen concentration within the gel, either 4 mg/ml or 6 mg/ml, so that the first digit is either “4” or “6”. The second digit refers to the volume of the collagen gel “4” for 4 ml, “5” for 5 ml, etc. The last two to three digits specify the percentage of the concentration of glutaraldehyde used to cross-link the collagen construct. For, example if a glutaraldehyde concentration of 0.005% was used, the last two to three digits in the device nomenclature would be “005”. As a final example a device labeled “6601” implies a construct made with 6 mg/ml collagen concentration with a volume of 6 ml cross-linked with 0.01% glutaraldehyde.

All final iterations of devices contain 6e6 hABMSCs cultured in TheraCol collagen hydrogels for 3 days in 6 well suspension culture plates (initial diameter 35 mm).

### Device Testing

Device testing was performed on the collagen-based, bioactive devices in this Example. The following device tests were performed: Physical Parameters, Collagenase Digestion, Bioactivity with VEGF ELISA and Mechanical Properties by Suture Retention Test.

### Results

### Device Production

Collagen concentration, collagen gel volume, and glutaraldehyde cross-linker concentration were altered to produce devices with different properties. Many of these variations are summarized in Tables 5 and b.

Preliminary assessments of the device were based on feasibility for manufacture as well as visual and tactile inspection. The following observations were made and altered the strategy and methods for next generation devices;
1. 4 mg/ml collagen gel constructs were smaller than 6 mg/ml.
2. The maximum gel volume tolerated in the 6 well plates was 9 ml with 4 ml of media for culture.
3. Gel volumes below 3 ml volumes did not fully gel or form solid constructs.
4. Glutaraldehyde cross-linking allowed increased durability in the handling of the constructs.
5. Cross-linking at any concentration produced a device that upon rehydration was much more durable than a non-cross-linked device.

Example of a glutaraldehyde cross-linked construct prior to dehydration is shown in FIG. 52. Actual measurements of the strength of the device iterations are discussed below.

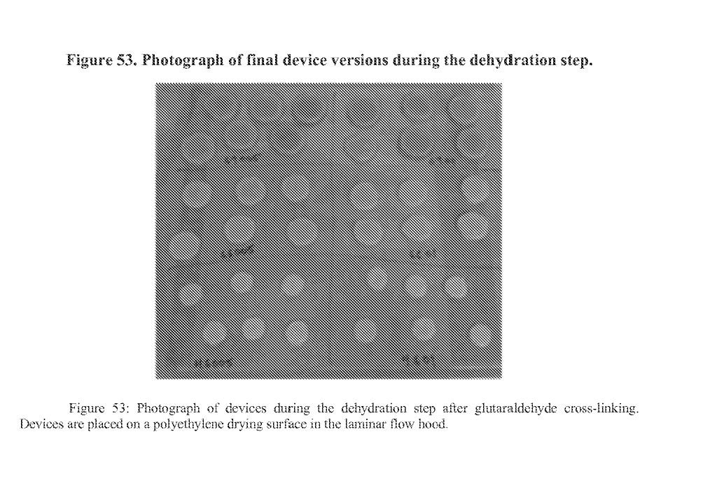

The final processing step of device production is the dehydration step. This was performed by allowing the cross-linked and washed construct to dehydrate on a polyethylene surface in the BSC under laminar air flow. FIG. 53 illustrates some device iterations during dehydration (at n=6 devices of 6 different iterations).

Several observations were made during the drying process:
1. The constructs with larger gel volumes are both thicker and larger in diameter than smaller gel volume constructs.
2. The larger and thicker constructs take longer than 12 hours to fully dehydrate. (NOTE: Full dehydration here refers to all liquid evaporation from the constructs under the conditions of ambient laminar air flow in the BSC. This does not, however, specify that the constructs have been dehydrated to a specific quantified humidity level below ambient air humidity.)
3. The 4 mg/ml collagen constructs were able to fully dehydrate in less than 12 hours.
4. Dehydration of the constructs led to complete evaporation of any visible liquid droplets with subsequent decrease in the height of the gels.
5. During dehydration, appearance of the surface went from clear and shiny to white and opaque upon full dehydration.
   Final fully dehydrated devices have a thickness less than 0.5 mm.

As mentioned, it is important to quench any remaining glutaraldehyde as unreacted residues can prove toxic to surrounding cells. One way to access the potential toxicity of fixed devices is to attempt to culture cells on top of the devices. Either hABM-SC or human chondrocytes was added on top of devices and their viability was observed over time in culture. FIG. 54 depicts the results of cytotoxicity of both chondrocytes and hABM-SCs after 4 days of culture on the surface of devices that had glycine quenching times of either 2 or 4 hours during the processing step of the device production. These results demonstrate the longer 4 hour glycine wash step allowed reduced toxicity of the glutaraldehyde cross-linked device. The devices having a 0.2 hour glycine wash time showed an increase in dead cells on the surface of the device after culture (red stain). The devices with a 4 hour glycine wash time had a high density of attached and live cells across the surface (green stain).

### Physical Properties of Final Dehydrated Devices

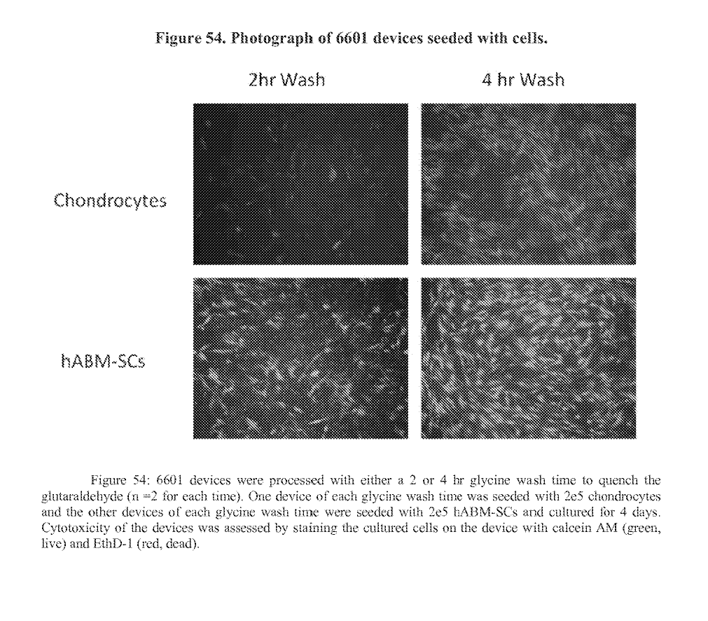

Many devices were not advanced to final testing because they did not meet the necessary criteria for the indication, the surgeons or feasibility for scale up. Six devices, listed in Table 6, were deemed acceptable and worthy of additional analysis. A photograph of five of the six final iterations of the bioactive collagen-based device is shown in FIG. 55.

These iterations all had a starting collagen concentration of 6 mg/ml within the construct. Variations in parameters such as collagen gel volume and glutaraldehyde concentrations of the 6 mg/ml collagen concentration constructs allowed significant variations in physical properties of the device configurations.

An increase in initial volume used to generate the collagen constructs results in increases of both diameter and weight of the device configuration. Visual illustration, as well as quantitative documentation, of these differences is provided in FIG. 55 and Table 6, respectively.

TABLE 6
Parameters of the final six devices.

| | | | | Processing |
|---|---|---|---|---|
| | Gel Construct Parameters | | | Parameters |
| Device | Collagen Conc. | Cell Number | Gel Volume | Glut. Conc. |
| 6501 | 6 mg/ml | 6e6 | 5 ml | 0.01% |
| 6601 | 6 mg/ml | 6e6 | 6 ml | 0.01% |
| 6701 | 6 mg/ml | 6e6 | 7 ml | 0.01% |
| 6505 | 6 mg/ml | 6e6 | 5 ml | 0.05% |
| 6705 | 6 mg/ml | 6e6 | 7 ml | 0.05% |
| 66005 | 6 mg/ml | 6e6 | 6 ml | 0.005% |

Table 6. The final six devices were created by varying the gel volume and glutaraldehyde concentration. This table summarizes the parameters and illustrates the numbering system used to name the devices. For each device the value in each column was combined in sequence to give the final name. For example 6501 was composed of “6” mg/ml collagen + “6” million cells + “5” mls of gel + “0.01” % glutaraldehyde.

TABLE 7
Table of physical properties of device iterations.

| Device version: | Diameter (mm) | Weight (mg) |
|---|---|---|
| 6501 | 24.2 ± 0.5 | 67.5 ± 9.1 |
| 6505 | 23.4 ± 0.5 | 64.9 ± 3.2 |
| 6701 | 30 ± 0.6 | 120.1 ± 14.0 |
| 6705 | 28.6 ± 1.5 | 113.8 ± 7.3 |
| 6601 | 27 ± 0* | 89.4 ± 2.6* |
| 66005 | 28.5 ± 0.7* | 99.3 ± 1.6* |

Table 7: Table of physical properties of diameters and dry weights of final 6 device iterations.
Diameters were measured of n = 5 devices.
Weights were measured of n = 3 devices.
*measurements taken on only n = 2 devices.

All six versions of the collagen-based, bioactive devices discussed in this section were similarly thin and flexible in the dehydrated state. Rehydration of the devices revealed differences in durability, specifically differing with the different glutaraldehyde cross-linking concentrations. Devices cross-linked with 0.05% were substantially more rigid, although still flexible. The 66005 device cross-linked with 0.005% glutaraldehyde was very flexible and pliable without falling apart after handling. Overall, these six devices ranged in pliability but had acceptable manageability and handling. The devices generated from 7 ml collagen gel volumes were noticeable thicker after rehydration compared to the 5 and 6 ml versions.

### Functional Properties of Devices

Bioactivity and strength of the devices were assessed and compared. Vascular endothelial growth factor, VEGF, is a relevant protein that can be therapeutically beneficial for tissue healing and regeneration and is expressed in high amounts by hABM-SC in culture. The amount of VEGF contained in the collagen gels was used as a surrogate marker for bioactivity of the devices.

Strength of the device configuration is important in the feasibility of handling, durability, and use of these devices as a potential product. Application of the device as a product to aid in healing or regeneration of a tissue would most likely include suturing of the device to the tissue and therefore strength was assessed by suture retention testing to compare the device iterations.

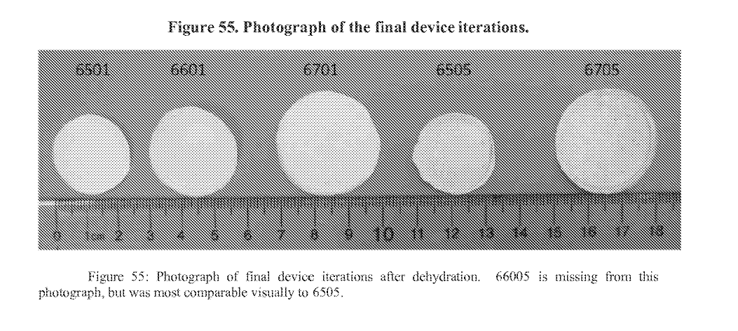

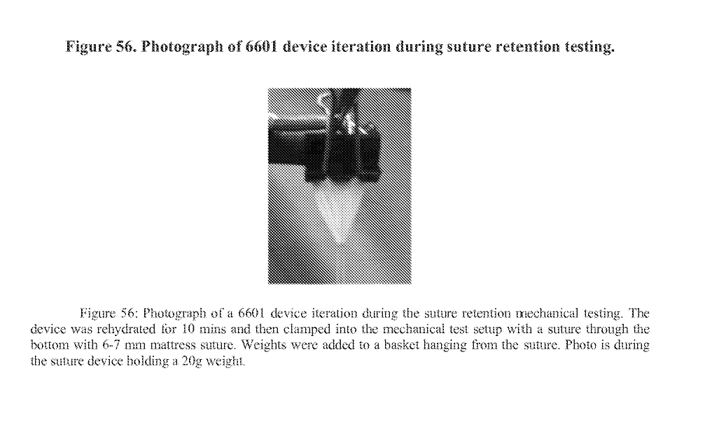

VEGF amounts contained in the devices were measured by digestion in collagenase with ELISA measurements performed on these collagenase digests. Suture retention testing was performed using standard laboratory equipment. The device was secured with a clamp at the top end and a mattress suture stitch through the bottom end. The suture was attached to a basket of weights. Strength of the devices was assessed by increasing weight until the device failed and the suture pulled out. A photograph of the 6601 device contained within the strength testing apparatus during a suture retention test while holding a 20 g weight is shown in FIG. 56.

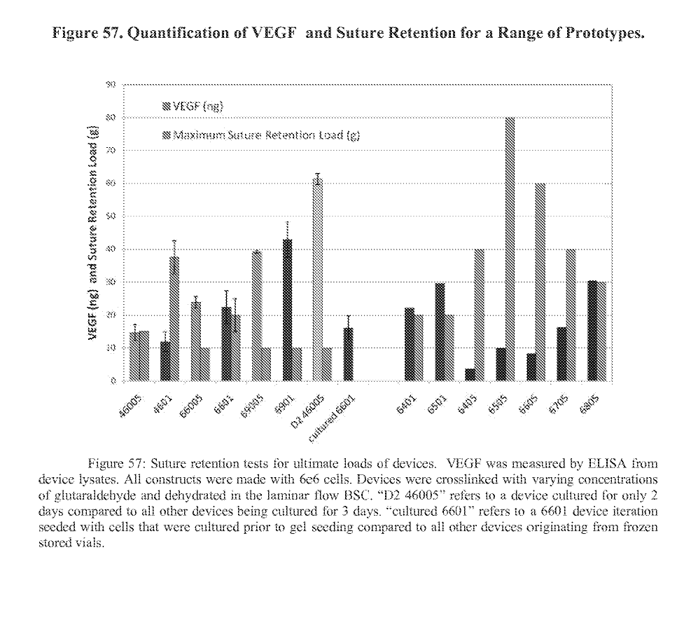

FIGS. 57 and 58 demonstrate the results for VEGF quantities (ng) contained within devices as well as maximum weight loads (g) the devices could withstand before failure during suture retention testing. FIG. 51 demonstrates these results for fifteen different device iterations with FIG. 58 representing the same data for only the final six iterations.

### Observations:

1. VEGF levels were lower in the device created from cultured cells compared to cryopreserved hABM-SC that were rapidly thawed and added to the collagen.
2. VEGF levels were higher in all devices made with 6 mg/ml collagen compared to 4 mg/ml.
3. The devices with 6 mg/ml collagen concentration cross-linked with either 0.005% or 0.01% glutaraldehyde contain on average VEGF amounts of 20 ng or higher.
4. The strongest of the 4 mg/ml collagen concentration devices, 46005 and 4601, contain less than 20 ng of VEGF.
5. Increasing gel volume, without addition of more cells or increasing collagen concentration, correlated with higher VEGF content.
6. There appears to be an inverse relationship between strength (glutaraldehyde concentration) and bioactivity; higher strength or concentration of glutaraldehyde correlates with lower bioactivity.
7. 6 mg/ml collagen cross-linked with 0.05% glutaraldehyde can withstand more weight than the devices cross-linked with 0.01%.
8. A trend appears showing decreasing strength with increasing the collagen gel volume above 5 ml.

A trade-off was observed between optimizing maximum strength with maximum bioactivity. Most devices with very high VEGF levels had poor strength, and ones with more strength had low (under 10 ng) VEGF levels.

FIG. 58 represents the same data from FIG. 57 but for only five of the six devices that were further considered as potential product candidates. Data for 6701 device was not completed because there were too few devices made of this iteration that were used instead for other studies. These device iterations represent devices that are feasible with manufacturing and acceptable with respect to achieving the initial target criteria; 1) very thin, 2) flexible, 3) durable 4) strong enough to hold a suture, 5) large enough to be cut to their desired size and shape.

Data and images for the device iterations are the shown in FIG. 55, Table 7, and FIG. 58. Notable differences and features of these devices:
Device iteration 66005 has the lower glutaraldehyde concentration of 0.005% which would allow the device to degrade more quickly in vivo than the other final iterations.
Devices 6501, 6601 and 6701 maintain good balance between strength and bioactivity with moderate levels of both,
Devices 6501, 6601 and 6701 differ in the device diameters and potential thicknesses after rehydration. 6501 has the smallest diameter and is the thinnest, while 6701 has the largest diameter and is thicker.

Devices 6505 and 6705 have higher glutaraldehyde cross-linking of 0.05% and therefore result in devices that are much stronger with bioactivity around 10 ng of VEGF.

### Example 22

### Testing of Collagen-Based, Bioactive Hand Tendon Repair Device

### Introduction:

The principal goals of this Example were to test the mechanical properties, including pliability, ease of use and handling of the collagen-based, bioactive devices of the present invention in a human cadaver model of hand flexor tendon repair. A secondary goal was to broadly discuss relevant biochemical and physical properties of the devices that could offer advantages in flexor tendon surgery. Lastly, the study evaluated the potential use of a PLGA-based device as a spacer in the CMC joint—this was also simulated using the same cadaveric limb.

### Methods:

One fresh frozen cadaver was utilized to simulate flexor tendon laceration and subsequent repair augmented with the devices of the invention. In a standard fashion, an extensile exposure was performed revealing the flexor tendon sheath, flexor tendons as well as the pulley system. Using a standard #15 blade, multiple flexor tendons were lacerated at various anatomic zones, including Verdan Zones 1-3 (Zone 1 being distal to the FDS insertion, Zone 2 proximal to the FDS insertion to the level of the metacarpo-phalangeal (MCP) joints, Zone 3 being proximal to the MCP joint to the distal extent of the transverse carpal ligament). The flexor tendons were repaired with a standard technique consisting of 4-0 Fiberwire with a modified Bunnell stitch. The devices were then placed around the repair circumferentially. They were either sutured in place with 6-0 Prolene or gently wrapped around the tendon to effect a complete circumferential covering. The tendons were then mobilized to ensure that the device did not displace away from the lacerated tendon site. Several devices were utilized and analyzed according to the ease of use, pliability, capacity to accept stitches, and ability to remain in place upon mobilization of the tendon.

### Results:

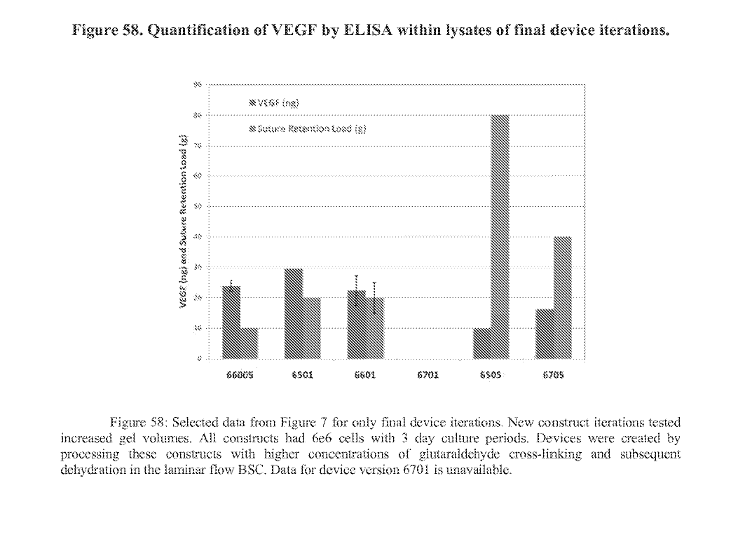

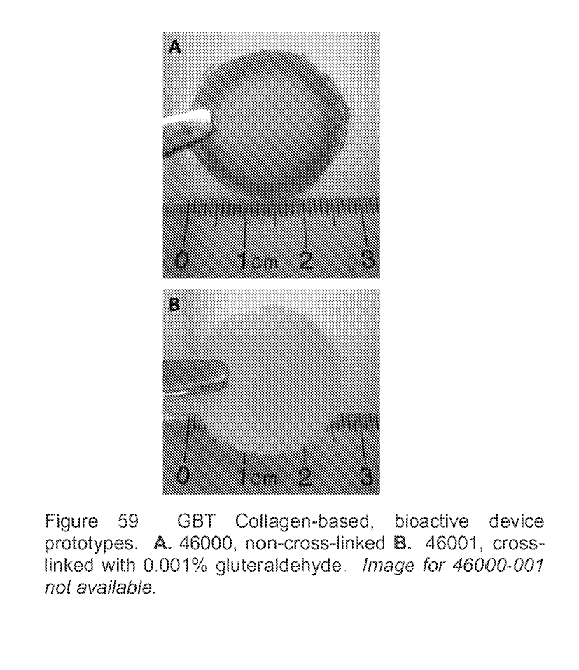

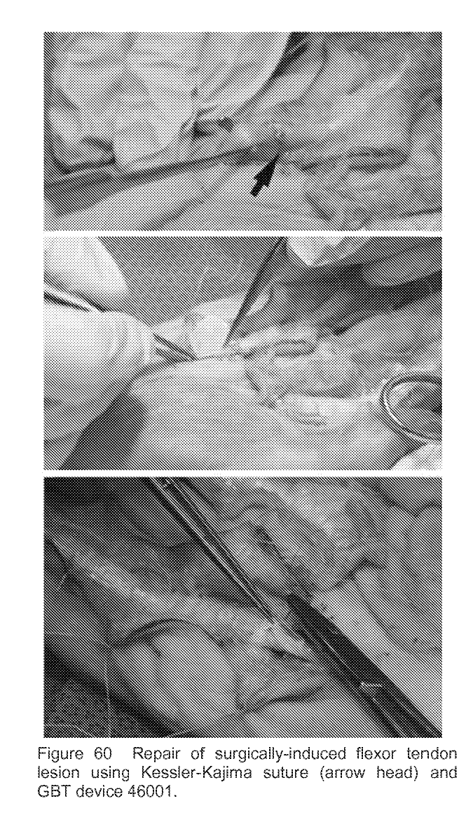

Based on testing of three separate collagen-based bioactive devices (46000, 46001, and 46000-001; FIG. 59), the first device (46001, crosslinked) handled easier than the later devices evaluated (46000, non-crosslinked and 46000-001, air-dried, crosslinked, air-dried). All three devices were able to accept sutures (Prolene, 6-0 caliber) that are typically used in an epi-tendinous repair. The device was successfully stitched to the tendon itself with the use of standard microsurgical technique without difficulty (FIG. 60). Though more difficult, the device could be stitched to itself.

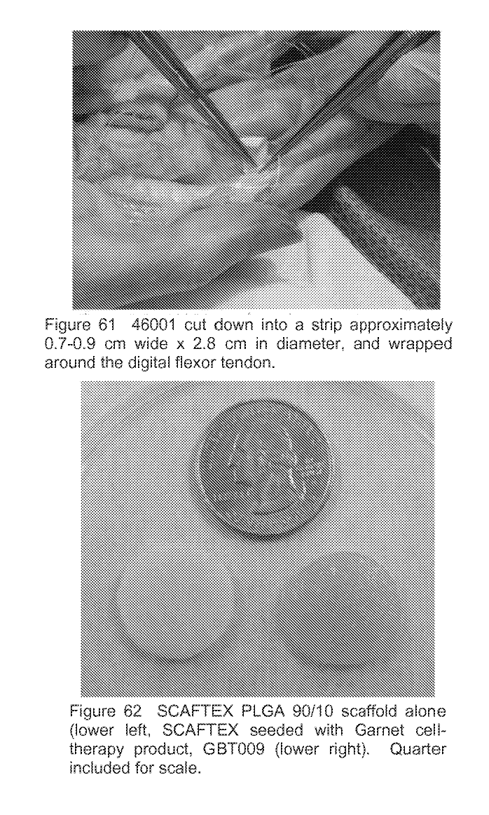

After submersing the devices in sterile water, all were pliable and able to conform to the tendon in a circumferential pattern. The non-crosslinked device 46000, however, became pasty and less solid with further manipulation, suggesting that, at least for the non-crosslinked device, pre-wetting before application may not be desirable. There was also kinking of the hydrated devices with active mobilization of the tendon, simulating early rehab protocols (i.e. place and hold or early active motion) often utilized within days after surgery. As a result, the concepts of applying the devices in their dry form and/or cutting the devices into strips were discussed as options to minimize “kinking and bunching” with early motion (FIG. 61). The devices were then tested in their dry form; unlike those that were pre-wet, these appeared to integrate well with the underlying tendon without bunching. In embodiments, in the early post-operative period when there is no active mobilization of the tendon, a product that has an immediate release of factors may be more effective than one that has a slower timed release. Should the device migrate from the area of repair, however, the local factors would be unable to act on the local tissue. In an embodiment, the device is laid down on the dorsal side of the tendon, possibly precluding the substrate from displacing on the volar surface.

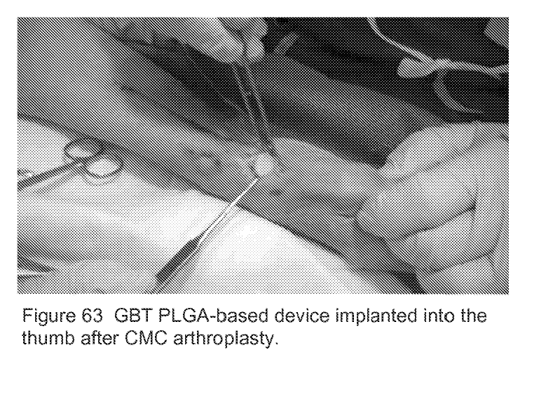

Current surgical treatment options for thumb arthritis surgery (CMC Arthroplasty) consist of a procedure in which either a portion of, or the entire trapezium is excised during thumb arthritis surgery. Various techniques and modifications have been described to treat this common form of arthritis. A device of the present invention (FIG. 62) was placed into the thumb CMC joint, following routine cadaveric dissection. The spacer appeared to be appropriately sized for this joint and fit well into the space (FIG. 63).

### Example 23

### Testing of Collagen-Based, Bioactive Hand Tendon Repair Device

### Introduction:

The principal goals of the study were to test the physical and mechanical properties, including size, shape, pliability, and handling of the collagen-based, bioactive devices according to the invention in a human cadaver model of hand flexor tendon repair. The primary goal of these tests was to assess multiple devices in order to identify a single preferred device that could offer advantages in flexor tendon surgery.

### Study Design:

Devices were first assessed on their overall physical appearance; size, shape, pliability, durability, ease of manipulation.

Devices that were deemed suitable based on preliminary physical assessments, were cut into strips and applied to a tendon.

Devices that could successfully be applied to or around a tendon, were evaluated on the ability to endure movement.

### Methods:

One fresh frozen cadaver was utilized to simulate flexor tendon laceration and subsequent repair augmented with implantable soft tissue medical devices of the invention. In a standard fashion, an extensile exposure was performed revealing the flexor tendon sheath, flexor tendons, as well as, the pulley system. Using a standard #15 blade, flexor tendons were lacerated at various anatomic zones, including Verdan Zones 1-3 (Zone 1 being distal to the FDS insertion, Zone 2 proximal to the FDS insertion to the level of the metacarpo-phalangeal (MCP) joints, Zone 3 being proximal to the MCP joint to the distal extent of the transverse carpal ligament). The flexor tendons were repaired with a standard technique consisting of 4-0 Fiberwire with a modified Bunnell stitch (FIG. 64). The devices were sutured into place at various levels (Zones 1-3) within the flexor tendon pulley system in an effort to reproduce the anatomic constraints that are often encountered. Specifically, the long finger flexor tendon was sectioned in the region of Zone 3, requiring both a primary flexor tendon repair in addition to augmentation with a device of the invention. The index finger was specifically prepared to accept a device in Zone 2, traditionally an area that represents a difficult repair due to the critical nature of its pulley systems, as well as the limited space to theoretically accept added bulk.

Prior to application, the circular devices were cut into three to four strips. The middle strips were used first. The device strips were applied to either repaired or normal tendons by wrapping around the circumference either with a straight overlapping configuration or with a “bowtie” non-overlapping configuration. In the straight overlapping application, the device was wrapped straight and completely around the tendon overlapping onto itself and secured with one stitch through the overlapping device end down to the device itself. The “bowtie” application was performed in some cases and included wrapping the device around the circumference of the tendon without overlap and instead laying the ends parallel to each other. In this configuration the device was secured to the tendon in three places with a stitch at each device end down to the tendon and one stitch in the middle holding together the adjacent pieces of the device. 6-0 Prolene sutures were used in securing the device to the tendon. The tendons were then mobilized to ensure that the device did not displace away from the lacerated tendon site. Several device iterations were utilized and assessed, both in dry and hydrated (10 minute hydration in water) states, according to the physical dimensions, ease of use, pliability, capacity to accept stitches, and ability to remain in place upon mobilization of the tendon.

### Results:

The following represents a review of six different collagen-based, bioactive devices (five iterations pictured in FIG. 65). Table 8 represents the overall evaluation of each device after assessment of dimensions, suture retention, appropriate in use and application for flexor tendon repair.

ABLE 8
Summary of results from testing all device prototype iterations both dry
and hydrated for acceptability of dimensions and suture retention after
application for flexor tendon repair (NP = not performed;
NC = no comment made).
Devices Applied Dry:

| | | | Application to | |
|---|---|---|---|---|
| Device | Diameter | Thickness | Tendon | Suture Retention |
| 6501 | smaller | acceptable | straight overlap | acceptable |
| 6601 | moderate | acceptable | bowtie | acceptable |
| 6701 | larger | acceptable | straight overlap | acceptable |
| 6505 | smaller | NC | NP | NP |
| 6705 | larger | acceptable | bowtie | acceptable |
| 66005 | moderate | acceptable | straight overlap and | poor to fair |
| | | | bowtie | |

Devices Applied After 10 min Rehydration:

| Device | Diameter | Thickness | Suture Retention |
|---|---|---|---|
| 6501 | too small | acceptable | NP |
| 6601 | good | acceptable | acceptable |
| 6701 | good | too thick | poor |
| 6505 | too small | NC | NP |
| 6705 | good | acceptable | acceptable |
| 66005 | NC | thin | NP; poor durability |

The following section discusses specific comments made during the testing of each of the six iterations of the devices tested during this study:

### 6501 Device:

The diameter of the dry 6501 device allowed complete wrapping around the resected flexor tendon of the long finger, in zone 3, but left no excess material for additional manipulation (FIG. 66). The diameter of the 6501 device in the wet state was still quite small, but the thickness was acceptable. The pliability and suture strength of this device was good.

### 6601 Device:

A strip of the 6601 device was applied to the tendon of the small finger in zone 3.

The diameter was very acceptable with a length allowing the bowtie configuration with excess material (shown in FIG. 67). Handling and pliability of the device in both the dry and hydrated states was acceptable during application of the device to the tendon. The 6601 device strip accepted sutures well both down to the tendon and to itself. This location in zone 3 of the small finger specifically has constrained space around the tendon, but the device was easily applied to the tendon and sutured with smooth gliding within the space. During mobilization of the tendon the device remained in place along the circumference and glided smoothly with the tendon.

### 6701 Device:

The 6701 device was able to be cut into multiple strips. A single dry strip was placed under the A2 pulley repair, surrounding the index finger flexor tendon, with subsequent closure of the sheath and was well tolerated with mobilization of finger with device remaining attached to the gliding tendon of zone 2 (FIG. 68a). The space available within this location along the flexor tendon of the index finger is specifically narrow and constrained, but the device was able to be applied and sutured to the tendon more than adequately with subsequent attachment remaining during gliding of pulley.

However, a rehydrated strip of the 6701 device applied to zone 3 of the middle finger became too thick and did not retain sutures well during application to the tendon (FIG. 68b). The diameter of the 6701 device was preferred allowing excess length after wrapping around tendon to allow additional manipulations.

### 6505 Device:

This device was observed to have a diameter similar to that of 6501, which was too small, and therefore the 6505 devices were not further tested. Application to tendon was not performed.

### 6705 Device:

Pliability of the 6705 device was good and thickness was satisfactory, both when dry and after hydration. The application of this device was very acceptable with flexibility during application around tendon. Was able to suture well with “bowtie” application to tendon in zone 3 of the index finger having each device strip end sutured to the tendon and a single suture through adjacent device pieces (three sutures depicted in FIG. 69). The space available along the flexor tendon of the index finger is specifically narrow and constrained, but the device was able to be applied and sutured to the tendon more than adequately.

### 66005 Device:

The diameter and thickness of the 66005 device, both dry and rehydrated, was very acceptable. A strip of the 66005 device in the dry state was applied to the tendon of the ring finger, zone 3, using the straight overlap configuration. Upon mobilization of the tendon, the device endured “bunching” and tore at the suture site displacing it away from the tendon. A second strip was applied using the bowtie configuration with suturing to the tendon. When the pulley system was employed, mobilization was acceptable.

### CONCLUSION

Based on testing of six separate collagen-based bioactive devices (6501, 6601, 6701, 6505, 6705, and 66005; FIG. 65—note 66005 missing from the photo), all were improved versions compared with devices assessed in Example 22. All second generation devices were more durable with improved handling, particularly in the hydrated state. Four of the six devices were well able to accept sutures (Prolene, 6-0 caliber) that are typically used in an epi-tendinous repair (one being not acceptable and one not tested). These four devices were successfully secured to the tendon without difficulty (both suturing to the tendon and to itself) and withstood mobilization of the tendons without displacement of the device away from the tendon. All devices applied to tendons of zones 2 and 3, including the constrained space of the index finger of zone 2 and small finger of zone 3, were easily able to fit within the space around the tendon during application and after application with mobilization of the tendons. Handling and application of the devices to the tendons with suturing was preferred in the dry state. Overall, the 6601 device iteration was the preferred device based on the aspects tested; device dimensions, pliability, handling, application and securing to tendon with sutures.

### REFERENCES

Cory et al., “Murine erythroid cell lines derived with c-myc retroviruses respond to leukemia-inhibitory factor, erythropoietin, and interleukin 3,” Cell Growth Differ. 2 (3):165-72 (1991).
Dexter et al., “Molecular and cell biological aspects of erythropoiesis in long-term bone marrow cultures,” Blood. 58(4):699-707 (1981).
Dia et al., “Human burst-forming units-erythroid need direct interaction with stem cell factor for further development,” Blood. 78(10):2493-7 (1991).
Hodohara et al., “Stromal cell-derived factor-1 (SDF-1) acts together with thrombopoietin to enhance the development of megakaryocytic progenitor cells (CFU-MK),” Blood. 95(3):769-75 (2000).
Miharada et al., “Efficient enucleation of erythroblasts differentiated in vitro from hematopoietic stem and progenitor cells,” Nature Biotech. 10:1255-56 (2006).
Müller-Ehmsen et al., “Rebuilding a damaged heart: long-term survival of transplanted neonatal rat cardiomyocytes after myocardial infarction and effect on cardiac function,” Circulation. 105(14):1720-6 (2002).
Nelson, “IL-2, Regulatory T-Cells, and Tolerance,” Jour. Immunol. 172: 3983-3988 (2004).
Quesenberry et al., “Studies on the regulation of hemopoiesis,” Exp. Hematol. 13: Suppl. 16:43-8 (1985).
Roecklein and Torok-Storb, “Functionally distinct human marrow stromal cell lines immortalized by transduction with the human papilloma virus E6/E7 genes,” Blood. 85(4): 997-1005 (1995).
Shao et al., “Effect of activin-A on globin expression in purified human erythroid progenitors,” Blood. February; 79(3):773-81 (1992).
Ullrich et al., “In vivo hematologic effects of recombinant interleukin-6 on hematopoiesis and circulating numbers of RBCs and WBCs,” Blood. 73(1): 108-10 (1989).

1-20. (canceled)

21. A population of equine bone marrow-derived cells, wherein the equine bone marrow-derived cells do not have multipotent differentiation capacity and are non-immortalized, wherein
(a) the equine bone marrow-derived cells express cell surface markers CD90, CD44 and GM-CSF and do not express high levels of CD34;
(b) the equine bone marrow-derived cells express cell surface markers CD90, CD44 and CD49d; or
(c) the equine bone marrow-derived cells were obtained by a process comprising:
(i) culturing equine bone marrow cells under a low oxygen condition or a low oxidative stress condition to produce an adherent cell population; and
(ii) culturing the adherent cell population at a seeding density of less than about 2,500 cells/cm² to produce the equine bone marrow-derived cells.

22. The population of equine bone marrow-derived cells of claim 21, wherein
(a) at least 99.6% of the equine bone marrow-derived cells of (a) or (b) express CD44;
(b) at least 96.5% of the equine bone marrow-derived cells of (a) or (b) express CD49d;
(c) at least 91.5% of the equine bone marrow-derived cells of (a) or (b) express CD49e;
(d) at least 87.2% of the equine bone marrow-derived cells of (a) or (b) express CD90;
(e) at least 97.1% of the equine bone marrow-derived cells of (a) or (b) express GM-CSF; or
(f) not more than 4.6% of the equine bone marrow-derived cells of (a) or (b) express CD34.

23. The population of equine bone marrow-derived cells of claim 21, wherein the low oxygen condition in (i) is between about 1 to 10% oxygen.

24. The population of the equine bone marrow-derived cells of claim 21, wherein in (i) the equine bone marrow cells are cultured at an initial seeding density of less than 60,000 cells/cm².

25. The population of equine bone marrow-derived cells of claim 21, wherein in (ii) the adherent cell population is cultured at a seeding density of less than about 2,500 cells/cm².

26. The population of equine bone marrow-derived cells of claim 21, wherein in (ii) the adherent cell population is cultured through at least about 15, at least about 20, at least about 25, at least about 30, at least about 35, or at least about 40 population doublings.

27. A method of culturing the equine bone marrow-derived cell population of claim 21, comprising:
(a) seeding a culture comprising a cell culture media with the equine bone marrow-derived cells; and
(b) maintaining the culture under conditions that allow growth of the equine bone marrow-derived cells.

28. The method of claim 27, wherein the seeding in (a) is at a seeding density of less than about 2,500 cells/cm².

29. The method of claim 27, wherein the conditions in (b) comprise low oxygen condition or low oxidative stress condition.

30. A conditioned media produced by a method comprising:
(a) the method of claim 27, and
(b) separating the equine bone marrow-derived cells from the cell culture media, wherein the cell culture media is the conditioned media.

31. A composition comprising a biocompatible or biodegradable matrix and a conditioned cell culture media produced by equine cells selected from the group comprising:
(a) equine cells that express cell surface markers CD90, CD44 and GM-CSF and do not express high levels of CD34; and
(b) equine cells that express cell surface markers CD90, CD44 and CD49d.

32. The composition of claim 31, wherein the equine cells are equine bone marrow derived cells that do not have multipotent differentiation capacity and are non-immortalized.

33. The composition of claim 32, wherein the equine bone marrow derived cells were obtained by a process comprising:
(i) culturing equine bone marrow cells under a low oxygen condition or a low oxidative stress condition to produce an adherent cell population; and
(ii) culturing the adherent cell population at a seeding density of less than about 2,500 cells/cm² to produce the equine bone marrow-derived cells.

34. The composition of claim 31, further comprising at least one therapeutic compound.

35. The composition of claim 31, wherein the matrix comprises collagen, polyglycolic acid, poly-lactic-co-glycolic acid, fibrin, hyaluronic acid, heparin, alginate, gelatin, chitosan, laminin, fibronectin, silicone, poly-tetrafluoroethylene, poly-dimethylsiloxane, polysulfones, polyethylene glycol, or polycaprolactone.

36. A method of preventing or repairing a medical condition in a horse in need thereof, comprising contacting the horse with a therapeutically effective amount of the composition of claim 31.

37. The method of claim 36, wherein the medical condition is an orthopedic injury.

38. The method of claim 37, wherein the orthopedic injury is a tendon injury.

39. A composition comprising a biocompatible or biodegradable matrix and the population of equine bone marrow-derived cells of claim 21.

40. The composition of claim 39, further comprising at least one therapeutic compound.

41. The composition of claim 39, wherein the matrix comprises collagen, polyglycolic acid, poly-lactic-co-glycolic acid, fibrin, hyaluronic acid, heparin, alginate, gelatin, chitosan, laminin, fibronectin, silicone, poly-tetrafluoroethylene, poly-dimethylsiloxane, polysulfones, polyethylene glycol, or polycaprolactone.

42. A method of preventing or repairing a medical condition in a horse in need thereof, comprising contacting the horse with a therapeutically effective amount of the composition of claim 39.

43. The method of claim 42, wherein the medical condition is an orthopedic injury.

44. The method of claim 43, wherein the orthopedic injury is a tendon injury.
